(19) United States
(12) Patent Application Publication
(10) Pub. No.: US 2023/0304062 A1
(43) Pub. Date: Sep. 28, 2023

(54) CONTROLLED TRANSCRIPTION OF POLYNUCLEOTIDES

(71) Applicant: Regeneron Pharmaceuticals, Inc., Tarrytown, NY (US)

(72) Inventors: Yu Zhao, Willison Park, NY (US); Michael Goren, Tarrytown, NY (US); Darya Burakov, Tarrytown, NY (US); Gang Chen, Yorktown Heights, NY (US)

(21) Appl. No.: 18/047,349

(22) Filed: Oct. 18, 2022

Related U.S. Application Data
(60) Provisional application No. 63/256,831, filed on Oct. 18, 2021.

(51) Int. Cl. C12P 21/02; C12N 5/00; C12N 15/86

(52) U.S. Cl. CPC C12P 21/02; C12N 5/00; C12N 15/86; C12N 2750/14143; C12N 2830/002

(57) ABSTRACT

Compositions and methods for the transcription of polynucleotide sequences encoding polypeptides in cells are provided. The cells and methods provided are useful for controlling expression of polypeptides for a wide variety of purposes.

This Application claims priority to U.S. Application Serial No. 63/256,831, filed Oct. 18, 2021, which is hereby incorporated by reference in its entirety.

### FIELD OF THE INVENTIONS

The present inventions described herein provide, among other things, cells, cell cultures, polynucleotide and polypeptide constructs, systems and methods for controlling the transcription of one or more polynucleotide sequences of interest. The inventions described herein further provide stable cell lines wherein the transcription of at least one polynucleotide (specifically a polydeoxyribonucleotide) sequence of interest can be tightly controlled in order to control expression of a polypeptide. When an RNA is not transcribed from DNA, a polypeptide cannot be translated from the RNA, which allows for control of protein expression by controlling transcription.

### REFERENCE TO ELECTRONIC SEQUENCE LISTING

The application contains a Sequence Listing, which has been submitted electronically in .XML format and is hereby incorporated by reference in its entirety. Said .XML copy, created on Oct. 5, 2022, is named “135975-66502.xml” and is 98,737 bytes in size. The sequence listing contained in this .XML file is part of the specification and is hereby incorporated by reference herein in its entirety

### BACKGROUND OF THE INVENTIONS

Various methods for controlled transcription of a polynucleotide sequence of interest in a cell are known to the art and described in U.S. Pat. o. 9,469,856. For example, No et al., Proc. Natl. Acad. Sci. USA 93:3346-3351 (1996) describe a controllable gene expression system utilizing a chimeric transactivator consisting of the ecdysone nuclear receptor fused to the VP16 transactivation domain from herpes simplex virus. Gossen et al., Proc. Natl. Acad. Sci. USA 89:5547-5551 (1992) describe a single system for controlling transcription of a polynucleotide sequence of interest based on a chimeric protein consisting of the tetracycline repressor protein fused with the VP16 transactivation domain. Gossen et al., Science 268: 1766-69 (1995) describes the fusion of a VP16 activation domain with a mutated ‘reverse’ Tet repressor that requires tetracycline for induction. Tetracycline-inducible gene expression is discussed in Ortiz and Johnson, Molec. Biochem. Parasitology 128: 43-40 (2003). Other described single control systems are cited in U.S. Patent No. 9,469,856, including Sadowski et al., Nature 335:563-564 (1988); Brent et al., Cell 40:729-736 (1985); Labow et al., Mol. Cell. Biol. 10:3342-3356 (1990), for example.

Problems resulting from leaky transcription related to the sole reliance on minimal promoters have led to systems using fusions of the steroid-binding domains of the glucocorticoid or estrogen nuclear receptors. See, for example, Mattioni et al., Methods Cell Biol. 43:335-352 (1994); Louvion et al. Gene 131:129-134 (1993); lida et al. J. Virol. 70: 6054-6059 (1996).

Further improvements in regulated expression systems are described and claimed in U.S. Pat. o. 9,469,856. However, ever tighter control of polynucleotide transcription and polypeptide expression is desired.

### SUMMARY OF THE INVENTIONS

The present inventions advantageously include and utilize regulatory fusion proteins (RFPs) (which can act as activators or repressors) and repressor proteins (which act as repressors), such as antibiotic repressors, and tandemly arranged operators to control transcription of at least one polynucleotide of interest. Transcription of a single polynucleotide of interest can be controlled according to the inventions or multiple polynucleotides in an operon-like arrangement can be controlled according to the inventions.

As employed herein, a first RFP can bind to a first operator, which can be located 5′ of a promoter and a polynucleotide of interest to be transcribed. A second RFP or a repressor protein can bind to a second operator. The second operator can be located 3′ of a promoter but 5′ of a polynucleotide to be transcribed. In some embodiments, the second operator can be located 3′ of a promoter but 5′ of a polynucleotide to be transcribed and another second operator optionally can be operably linked to a polynucleotide encoding the first RFP or a repressor protein.

The descriptions of aspects and embodiments of the inventions provide methods for controlling the transcription of a polynucleotide of interest in a cell, wherein the method comprises (I) maintaining a cell in a medium without an effective amount of a ligand of both an activator and a repressor, wherein the cell comprises: (A) a promoter operably linked to a polynucleotide of interest and controlled by a first operator operably linked and positioned 5′ with respect to the promoter; (B) a polynucleotide encoding the activator; (C) a second operator; and (D) a polynucleotide encoding the repressor, wherein transcription of the polynucleotide of interest is inhibited in the absence of the ligand of both the activator and the repressor; and (II) controlling the cell to transcribe the polynucleotide of interest by maintaining the cell in a medium with an effective amount of the ligand of both the activator and the repressor. The second operator can be operably linked and positioned 3′ with respect to the promoter and 5′ with respect to the polynucleotide of interest. The activator can bind to the first operator in the presence of the ligand to permit transcription of the polynucleotide of interest. The repressor can be a repressor protein, such as an antibiotic repressor, wherein transcription of the polynucleotide of interest is inhibited in the absence of the ligand, and wherein transcription is permitted in the presence of the ligand. The repressor protein can bind to the second operator in the absence of the ligand. The activator can be a regulatory fusion protein (RFP). The ligand can be selected from the group consisting of tetracycline and doxycycline. An activator RFP can be a reverse tetracycline transactivator. A repressor protein can be an antibiotic repressor, such as a tetracycline repressor.

Further provided are methods for controlling the transcription of a polynucleotide of interest in a cell, wherein the method comprises (I) maintaining a cell in a medium without an effective amount of a ligand of an activator (activator ligand) and with an effective amount of ligand of a repressor (repressor ligand), wherein the cell comprises: (A) a promoter operably linked to a polynucleotide of interest and controlled by a first operator operably linked and positioned 5′ with respect to the promoter; (B) a polynucleotide encoding the activator; (C) a second operator; and (D) a polynucleotide encoding the repressor, wherein transcription of the polynucleotide of interest is inhibited in the absence of the activator ligand and the presence of the repressor ligand; and (II) controlling the cell to transcribe the polynucleotide of interest by maintaining the cell in a medium with an effective amount of the activator ligand and without an effective amount of the repressor ligand. The second operator can be operably linked and positioned 3′ with respect to the promoter and 5′ with respect to the polynucleotide of interest. The activator can bind to the first operator in the presence of the activator ligand to permit transcription of the polynucleotide of interest. The activator can be a regulatory fusion protein (RFP). The repressor can be a regulatory fusion protein (RFP), wherein transcription of the polynucleotide of interest is inhibited in the presence of the repressor ligand, and transcription is permitted in the absence of the repressor ligand. An activator RFP can be a reverse tetracycline transactivator. The activator ligand can be selected from the group consisting of tetracycline and doxycycline. A repressor RFP can be ArcEr, and the repressor RFP ligand can be selected from the group consisting of estrogen, estradiol (E2), tamoxifen and 4-hydroxytamoxifen (OHT).

There also are provided methods for controlling the transcription of a polynucleotide of interest in cell culture, wherein the methods comprise: I. maintaining at least one cell in a medium with or without an effective amount of a first ligand of a first regulatory fusion protein (RFP) and with an effective amount of a second ligand of a second RFP, wherein the cell comprises: (A) a promoter operably linked to a polynucleotide of interest and controlled by a first operator operably linked and positioned 5′ with respect to the promoter; (B) a polynucleotide encoding a first RFP, where the first RFP comprises: (1) a transcription activating domain fused to a first DNA binding domain; and (2) a ligand-binding domain; wherein the first ligand is capable of binding to the ligand-binding domain of the first RFP, and wherein the DNA binding domain of the first RFP is capable of binding to the operator positioned 5′ when in the presence of the first ligand; (C) a second operator; and (D) a polynucleotide encoding the second RFP that differs from the first RFP, wherein the second RFP comprises: (1) a second DNA binding domain; and (2) a ligand-binding domain; wherein the second ligand is capable of binding to the ligand-binding domain of the second RFP, and wherein the second RFP is capable of binding to the second operator in the presence of the second ligand; wherein transcription of the polynucleotide of interest is inhibited in the absence of an effective amount of the first ligand and in the presence of an effective amount of the second ligand; and (II) controlling the cell to transcribe the polynucleotide of interest by maintaining the cell in a medium with an effective amount of the first ligand and without an effective amount of the second ligand. The second operator can be operably linked and positioned 3′ with respect to the promoter and 5′ with respect to the polynucleotide sequence encoding the protein of interest. Another second operator optionally can be operably linked to the polynucleotide sequence encoding the first RFP. The first RFP as an activator can be a reverse tetracycline transactivator (rtTA). The second RFP as a repressor can comprise an Arc repressor binding domain fused to the estrogen receptor ligand binding domain (ArcEr). The first operator can be a Tet Response Element (TRE). The second operator can be an Arc operator (AO). The cells can further comprise a repressor that is altered by the first ligand. The repressor can be a tet repressor protein (TetR). Additionally, the polynucleotide encoding the first RFP can be operably linked to promoter and optionally a second Arc operator. The promoter can be a CMV promoter, such as CMVmin. ArcEr can control the transcription of the polynucleotide encoding rtTA.

There also are provided methods for controlling the transcription of polynucleotides of interest in cell culture, wherein the methods comprise: (I) maintaining at least one cell in a medium with or without an effective amount of a first ligand of a first regulatory fusion protein (RFP) and with an effective amount of a second ligand of a second RFP, wherein the cell comprises: (A) a promoter operably linked to a polynucleotide of interest and controlled by Tet Response Element (TRE) operably linked and positioned 5′ with respect to the promoter; (B) a polynucleotide encoding a first RFP, where the first RFP comprises: (1) a transcription activating domain fused to a DNA binding domain; and (2) a ligand-binding domain, wherein the first ligand is capable of binding to the ligand-binding domain of the first RFP, and wherein the DNA binding domain of the first RFP is capable of binding to the operator positioned 5′ when in the presence of the first ligand; (C) an Arc operator operably linked and positioned 3′ with respect to the promoter and 5′ with respect to the polynucleotide encoding the protein of interest; and (D) a polynucleotide encoding the second RFP, wherein the second RFP comprises: (1) an Arc repressor DNA-binding domain; and (2) a ligand-binding domain; wherein the second ligand is capable of binding to the ligand-binding domain of the second RFP, and wherein the second RFP is capable of binding to the Arc operator in the presence of the second ligand; wherein transcription of the polynucleotide encoding the protein of interest is inhibited in the absence of an effective amount the first ligand and in the presence of an effective amount of the second ligand; and (II) controlling the cell to transcribe the polynucleotide of interest by maintaining the cell in a medium with an effective amount of the first ligand and without an effective amount of the second ligand. The first ligand can be selected from the group consisting of tetracycline, doxycycline and derivatives thereof. The second ligand can be selected from the group consisting of estrogen, estradiol (E2), tamoxifen, 4-hydroxytamoxifen (OHT) and derivatives thereof. The ligand-binding domain of the second RFP can be the ligand binding domain of a steroid receptor. The first regulatory fusion protein (RFP) as an activator can be a reverse tetracycline transactivator (rtTA). The second RFP as a repressor can be ArcER, which has the Arc repressor binding domain fused to the estrogen receptor ligand binding domain (Arc is a repressor from phage P22). The promoter operably linked to the polynucleotide sequence encoding a polypeptide of interest can be a CMV promoter, such as a CMVmin promoter. A CMV promoter and an Arc operator optionally can be operably linked to the polynucleotide encoding the first RFP. An SV40 E/L promoter, or other constitutive promoter, can be operably linked to the polynucleotide encoding the second RFP.

Moreover, there are provided methods for controlling the transcription of a polynucleotide of interest in a cell, wherein the method comprises (I) maintaining at least one cell in a medium without an effective amount of a ligand of a regulatory fusion protein (RFP) and a repressor protein, wherein the cell comprises: (A) a promoter operably linked to a polynucleotide of interest and controlled by a first operator operably linked and positioned 5′ with respect to the promoter; (B) a polynucleotide encoding the RFP, wherein the RFP comprises: (1) a transcription activating domain fused to a DNA binding domain; and (2) a ligand-binding domain; wherein the ligand is capable of binding to the ligand-binding domain of the RFP, and wherein the DNA binding domain of the RFP is capable of binding to the first operator when in the presence of the ligand; (C) a second operator; and (D) a polynucleotide encoding the repressor protein, wherein the repressor protein can bind to the second operator only in the absence of the ligand, wherein transcription of the polynucleotide is inhibited in the absence of an effective amount of the ligand; and (II) controlling the cell to transcribe the polynucleotide of interest by maintaining the cell in a medium with an effective amount of the ligand. The second operator can be operably linked and positioned 3′ with respect to the promoter and 5′ with respect to the polynucleotide of interest. The repressor protein can bind to the second operator in the absence of the ligand to inhibit transcription of the polynucleotide of interest. The RFP can bind to the first operator in the presence of the ligand to permit transcription of the polynucleotide of interest. The ligand can be selected from the group consisting of tetracycline and doxycycline. The activator RFP can be a reverse tetracycline transactivator (rtTA). The repressor protein can be a tetracycline repressor (TetR). The first operator can be a Tet Response Element (TRE). The second operator can be a Tet operator.

Additionally, there are provided methods for controlling the transcription of polynucleotides of interest in cell culture, wherein the methods comprise: (I) maintaining at least one cell in a medium with or without an effective amount of a first ligand of a first regulatory fusion protein (RFP) and with an effective amount of a second ligand of a second RFP, wherein the cell comprises: (A) a promoter operably linked to a polynucleotide of interest and controlled by a Tet Response Element (TRE) operably linked and positioned 5′ with respect to the promoter; (B) a polynucleotide encoding a first RFP, wherein the first RFP comprises: (1) a transcription activating domain fused to a DNA binding domain; and (2) a ligand-binding domain; wherein the first ligand is capable of binding to the ligand-binding domain of the first RFP, and wherein the DNA binding domain of the first RFP is capable of binding to the TRE positioned 5′ when in the presence of the first ligand; (C) a Tet operator operably linked and positioned 3′ with respect to the promoter and 5′ with respect to the polynucleotide of interest; and (D) a polynucleotide encoding the second RFP, wherein the second RFP comprises: (1) an Arc repressor DNA-binding domain; and (2) a ligand-binding domain; wherein the second ligand is capable of binding to the ligand-binding domain of the second RFP, and wherein the second RFP is capable of binding to the Arc operator in the presence of the second ligand; wherein transcription of the polynucleotide is inhibited in the absence of an effective amount of the first ligand and the presence of an effective amount of the second ligand; and (II) controlling the cell to transcribe the polynucleotide of interest by maintaining the cell in a medium with an effective amount of the first ligand and without an effective amount of the second ligand. The first ligand can be selected from the group consisting of tetracycline, doxycycline and derivatives thereof. The second ligand can be selected from the group consisting of estrogen, estradiol (E2), tamoxifen, 4-hydroxytamoxifen (OHT) and derivatives thereof. The ligand-binding domain of the second RFP can be the ligand binding domain of a steroid receptor. The first regulatory fusion protein (RFP) as an activator can be a reverse tetracycline transactivator (rtTA). The second RFP as a repressor can be ArcER. The promoter operably linked to the polynucleotide sequence encoding a polypeptide of interest can be a CMV promoter, such as CMVmin. A CMV promoter and an Arc operator optionally can be operably linked to the polynucleotide encoding the first RFP. An SV40 E/L promoter, or other constitutive promoter, can be operably linked to the polynucleotide encoding the second RFP. The cells can further comprise a polynucleotide encoding a repressor that is altered by the first ligand. The repressor can be TetR.

There also are provided methods for controlling the transcription of a polynucleotide of interest in cell culture, wherein the methods comprise: maintaining at least one cell in a medium with or without an effective amount of a first ligand of a first regulatory fusion protein (RFP) and with an effective amount of a second ligand of a second RFP, wherein the cell comprises (A) a promoter; (B) an Arc operator; and (C) a polynucleotide encoding a reverse tetracycline transactivator fusion protein (rtTA), wherein (A), (B) and (C) are operably linked, and wherein transcription of the rtTA polynucleotide is controlled by a fusion protein comprising an Arc repressor binding domain and an estrogen receptor ligand binding domain (ArcEr); wherein rtTA can control the transcription of a polynucleotide of interest. The promoter can be a CMV promoter, such as CMVmin. The first ligand can be selected from the group consisting of tetracycline and doxycycline and derivatives thereof. The second ligand can be selected from the group consisting of estrogen, estradiol (E2), tamoxifen, 4-hydroxytamoxifen (OHT) and derivatives thereof.

The inventions also provide cells capable of controlled transcription of at least one polynucleotide of interest, wherein a cell comprises: (A) a promoter operably linked to a polynucleotide of interest and controlled by a first operator operably linked and positioned 5′ with respect to the promoter; (B) a polynucleotide encoding an activator; (C) a second operator; and (D) a polynucleotide encoding a repressor, wherein transcription of the polynucleotide of interest is inhibited in the absence of a ligand of both the activator and the repressor, and is permitted in the presence of the ligand of both the activator and the repressor. The second operator can be operably linked and positioned 3′ with respect to the promoter and 5′ with respect to the polynucleotide of interest. The activator can bind to the first operator in the presence of the ligand to permit transcription of the polynucleotide of interest. The repressor can be a repressor protein, wherein transcription of the polynucleotide of interest is inhibited in the absence of the ligand, and wherein transcription is permitted in the presence of the ligand. The repressor protein can bind to the second operator in the absence of the ligand. The activator can be a regulatory fusion protein (RFP). The ligand can be selected from the group consisting of tetracycline and doxycycline. The activator RFP can be a reverse tetracycline transactivator. The repressor protein can be a tetracycline repressor.

Additionally, there are provided cells capable of controlled transcription of at least one polynucleotide of interest, wherein a cell comprises: (A) a promoter operably linked to a polynucleotide of interest and controlled by a first operator operably linked and positioned 5′ with respect to the promoter; (B) a polynucleotide encoding a first regulatory fusion protein (RFP), where the first RFP comprises: (1) a transcription activating domain fused to a first DNA binding domain; and (2) a ligand-binding domain; wherein the first ligand is capable of binding to the ligand-binding domain of the first RFP, and wherein the DNA binding domain of the first RFP is capable of binding to the operator positioned 5′ when in the presence of the first ligand; (C) a second operator; and (D) a polynucleotide encoding the second RFP that differs from the first RFP, wherein the second RFP comprises: (1) a second DNA-binding domain; and (2) a ligand-binding domain; wherein the second ligand is capable of binding to the ligand-binding domain of the second RFP, and wherein the second RFP is capable of binding to the second operator in the presence of the second ligand; wherein transcription of the polynucleotide is inhibited in the absence of the first ligand and in the presence of the second ligand and is permitted in the presence of the first ligand and absence of the second ligand. The second operator can be operably linked and positioned 3′ with respect to the promoter and 5′ with respect to the polynucleotide sequence encoding the protein of interest. The second operator optionally can be operably linked to the polynucleotide sequence encoding the first RFP. The cells can comprise a polynucleotide that encodes the repressor that is altered by the first ligand. The repressor can be TetR. The polynucleotide (B) encoding the first RFP can be operably linked to promoter and a second Arc operator. The promoter can be a CMV promoter, such as CMVmin. The first RFP as an activator can be a reverse tetracycline transactivator fusion protein (rtTA) and the second RFP as a repressor can be a fusion protein comprising an Arc repressor binding domain and an estrogen receptor ligand binding domain (ArcEr). ArcEr can control the transcription of the polynucleotide encoding rtTA.

Furthermore, there are provided cells capable of controlled transcription of a polynucleotide of interest, wherein a cell comprises (A) a promoter operably linked to a polynucleotide of interest and controlled by a first operator operably linked and positioned 5′ with respect to the promoter; (B) a polynucleotide encoding an activator; (C) a second operator; and (D) a polynucleotide encoding a repressor; wherein transcription of the polynucleotide of interest is inhibited in the absence of an effective amount if an activator ligand and the presence of an effective amount of a repressor ligand; and permitted in the presence of an effective amount of the activator ligand and the absence of an effective amount of the repressor ligand. The second operator can be operably linked and positioned 3′ with respect to the promoter and 5′ with respect to the polynucleotide of interest. The activator can bind to the first operator in the presence of the activator ligand to permit transcription of the polynucleotide of interest. The activator can be a regulatory fusion protein (RFP). The repressor can be a regulatory fusion protein (RFP), wherein transcription of the polynucleotide of interest is inhibited in the presence of the repressor ligand, and transcription is permitted in the absence of the repressor ligand. The activator RFP can be a reverse tetracycline transactivator. The activator ligand can be selected from the group consisting of tetracycline and doxycycline. The repressor RFP can be ArcEr. The repressor ligand can be selected from the group consisting of estrogen, estradiol (E2), tamoxifen and 4-hydroxytamoxifen (OHT).

There also are provided cells capable of controlled transcription of at least one polynucleotide of interest, wherein a cell comprises: (A) a promoter operably linked to a polynucleotide of interest and controlled by Tet Response Element (TRE) operably linked and positioned 5′ with respect to the promoter; (B) a polynucleotide encoding a first regulatory fusion protein (RFP), where the first RFP comprises: (1) a transcription activating domain fused to a DNA binding domain; and (2) a ligand-binding domain; wherein the first ligand is capable of binding to the ligand-binding domain of the first RFP, and wherein the DNA binding domain of the first RFP is capable of binding to the operator positioned 5′ when in the presence of the first ligand; (C) an Arc operator operably linked and positioned 3′ with respect to the promoter and 5′ with respect to the polynucleotide encoding the protein of interest; and (D) a polynucleotide encoding the second RFP, wherein the second RFP comprises: (1) an Arc repressor DNA-binding domain; and (2) a ligand-binding domain; wherein the second ligand is capable of binding to the ligand-binding domain of the second RFP, and wherein the second RFP is capable of binding to the Arc operator in the presence of the second ligand; wherein transcription of the polynucleotide is inhibited in the absence of the first ligand and in the presence of the second ligand and is permitted in the presence of the first ligand and absence of the second ligand. The first ligand can be selected from the group consisting of tetracycline, doxycycline and derivatives thereof. The second ligand can be selected from the group consisting of estrogen, estradiol (E2), tamoxifen, 4-hydroxytamoxifen (OHT) and derivatives thereof. The ligand-binding domain of the second RFP can be the ligand binding domain of a steroid receptor. The first regulatory fusion protein (RFP) as an activator can be a reverse tetracycline transactivator (rtTA). The second RFP as a repressor can be ArcER. The promoter operably linked to the polynucleotide sequence encoding a polypeptide of interest can be a CMV promoter, such as a CMVmin promoter. A CMV promoter and an Arc operator optionally can be operably linked to the polynucleotide encoding the first RFP. An SV40 E/L promoter, or other constitutive promoter, can be operably linked to the polynucleotide encoding the second RFP.

In addition, there are provided cells capable of controlled transcription of a polynucleotide of interest, wherein a cell comprises (A) a promoter operably linked to a polynucleotide of interest and controlled by a first operator operably linked and positioned 5′ with respect to the promoter; (B) a polynucleotide encoding a regulatory fusion protein (RFP), wherein the RFP comprises: (1) a transcription activating domain fused to a DNA binding domain; and (2) a ligand-binding domain; wherein the ligand is capable of binding to the ligand-binding domain of the RFP, and wherein the DNA binding domain of the RFP is capable of binding to the first operator when in the presence of the ligand; (C) a second operator; and (D) a polynucleotide encoding a repressor protein, wherein the repressor protein can bind to the second operator only in the absence of the ligand, wherein transcription of the polynucleotide of interest is inhibited in the absence of an effective amount of the ligand of both the activator and the repressor, and is permitted in the presence of an effective amount of the ligand of both the activator and the repressor. The second operator can be operably linked and positioned 3′ with respect to the promoter and 5′ with respect to the polynucleotide of interest. The repressor protein can bind to the second operator in the absence of the ligand to inhibit transcription of the polynucleotide of interest. The RFP can bind to the first operator in the presence of the ligand to permit transcription of the polynucleotide of interest. The ligand can be selected from the group consisting of tetracycline and doxycycline. The activator RFP can be a reverse tetracycline transactivator (rtTA). The repressor protein can be a tetracycline repressor (TetR). The first operator can be a Tet Response Element (TRE). The second operator can be a Tet operator.

Additionally, there are provided cells capable of controlled transcription of at least one polynucleotide of interest, wherein a cell comprises: (A) a promoter operably linked to a polynucleotide of interest and controlled by a Tet Response Element (TRE) operably linked and positioned 5′ with respect to the promoter; (B) a polynucleotide encoding a first regulatory fusion protein (first RFP), where the first RFP comprises: (1) a transcription activating domain fused to a DNA binding domain; and (2) a ligand-binding domain; wherein the first ligand is capable of binding to the ligand-binding domain of the first RFP, and wherein the DNA binding domain of the first RFP is capable of binding to the TRE positioned 5′ when in the presence of the first ligand; (C) a Tet operator operably linked and positioned 3′ with respect to the promoter and 5′ with respect to the polynucleotide of interest; and (D) a polynucleotide encoding the second RFP, wherein the second RFP comprises: (1) an Arc repressor DNA-binding domain; and (2) a ligand-binding domain; wherein the second ligand is capable of binding to the ligand-binding domain of the second RFP, and wherein the second RFP is capable of binding to the Arc operator in the presence of the second ligand; wherein transcription of the polynucleotide is inhibited in the absence of the first ligand and in the presence of the second ligand and is permitted in the presence of the first ligand and absence of the second ligand. The first ligand can be selected from the group consisting of tetracycline, doxycycline and derivatives thereof. The second ligand can be selected from the group consisting of estrogen, estradiol (E2), tamoxifen, 4-hydroxytamoxifen (OHT) and derivatives thereof. The first regulatory fusion protein (RFP) as an activator can be a reverse tetracycline transactivator (rtTA). The second RFP as a repressor can be ArcER. The promoter operably linked to the polynucleotide sequence encoding a polypeptide of interest can be a CMV promoter, such as a CMVmin. A CMV promoter and an Arc operator optionally can be operably linked to the polynucleotide encoding the first RFP. An SV40 E/L promoter, or other constitutive promoter, can be operably linked to the polynucleotide encoding the second RFP. The cell can further comprise a polynucleotide encoding a repressor that is altered by the first ligand. The repressor can be TetR.

Additionally, there are provided cells capable of controlled transcription of at least one polynucleotide of interest when present, wherein a cell comprises: (A) a polynucleotide sequence encoding a first regulatory fusion protein (first RFP), where the first RFP comprises: (1) a transcription activating domain fused to a DNA binding domain; and (2) a ligand-binding domain; wherein the first ligand is capable of binding to the ligand-binding domain of the first RFP, and wherein the DNA binding domain of the first RFP is capable of binding to the operator positioned 5′ when in the presence of the first ligand; (B) a polynucleotide sequence encoding the second regulatory fusion protein (second RFP), wherein the second RFP comprises: (1) a DNA binding domain comprising an Arc repressor DNA-binding domain; and (2) a ligand-binding domain; wherein the second ligand is capable of binding to the ligand-binding domain of the second RFP, and wherein the second RFP is capable of binding to an Arc operator in the presence of the second ligand; (C) one or more insertion sites for a polynucleotide of interest that is operably linked to a promoter and at least one operator. The first ligand can be selected from the group consisting of tetracycline, doxycycline and derivatives thereof. The second ligand can be selected from the group consisting of estrogen, estradiol (E2), tamoxifen, 4-hydroxytamoxifen (OHT) and derivatives thereof. The first regulatory fusion protein (RFP) as an activator can be a reverse tetracycline transactivator (rtTA). The second RFP as a repressor can be ArcER. The promoter operably linked to the polynucleotide sequence encoding a polypeptide of interest can be a CMV promoter, such as a CMVmin promoter. A CMV promoter and an Arc operator optionally can be operably linked to the polynucleotide encoding the first RFP. An SV40 E/L promoter or other constitutive promoter can be operably linked to the polynucleotide encoding the second RFP. A cell can further comprise a polynucleotide encoding a repressor that is altered by the first ligand. The repressor can be TetR.

There also are provided cells capable of controlling the transcription of a polynucleotide of interest, wherein a cell comprises (A) a promoter; (B) an Arc operator; and (C) a polynucleotide encoding a reverse tetracycline transactivator fusion protein (rtTA), wherein (A), (B) and (C) are operably linked, and wherein transcription of the rtTA polynucleotide is controlled by a fusion protein comprising an Arc repressor binding domain and an estrogen receptor ligand binding domain (ArcEr); wherein rtTA can control the transcription of a polynucleotide of interest. The promoter can be a CMV promoter, such as CMVmin. The first ligand can be selected from the group consisting of tetracycline and doxycycline. The second ligand can be selected from the group consisting of estrogen, estradiol (E2), tamoxifen, 4-hydroxytamoxifen (OHT) and derivatives thereof.

There also are provided master cell banks, working cell banks, developmental cell banks, cell cultures, seed cultures, and production cultures comprising cells according to the inventions, as well as bioreactors and fermenters containing cell cultures comprising cells according to the inventions described herein.

Aspects of the embodiments include: maintaining the cells in absence of a first ligand and in the presence of a second ligand, or alternatively in the presence of the first ligand as well. Under these maintenance conditions, the percentage of cells comprising copies of the DNA polynucleotide sequence encoding the polypeptide of interest has reduced less than about 5%. Under the same maintenance conditions, the expression of the polypeptide of interest can be at least 50% less, at least 60% less, at least 70% less, at least 80% less, at least 90% less, or at least 95% less than the expression of the polypeptide in the cells in the presence of the first ligand and the absence of the second ligand after a time period sufficient to allow the second ligand previously-present to clear, usually about 4 to 14 days depending on the second ligand and culture conditions. Under the same maintenance conditions, the number of RNA copies encoding the polypeptide of interest can be at least 70% less, at least 75% less, at least 80% less, at least 85% less, at least 90% less, or at least 95% less than the number of RNA copies encoding the polypeptide in the cells in the presence of the first ligand and the absence of the second ligand previously-present after a time period sufficient to allow the second ligand to clear, usually about 4 to 14 days depending on the second ligand and culture conditions.

In certain embodiments, the transcription of the polynucleotide sequence encoding the first regulatory fusion protein (first RFP) is inhibited in the presence of the second ligand, such as OHT, and second RFP, such as ArcER.

In certain embodiments, the promoter operably linked to the polynucleotide sequence encoding the protein of interest is a CMV promoter. The promoter may be a CMVmin promoter.

In certain embodiments, the polynucleotide sequence of interest encodes a polypeptide and/or product of interest. The polynucleotide sequence of interest can encode a polypeptide of interest. The polypeptide of interest can be a protein that is toxic or inhibitory to the cell, such as a viral protein.

In certain embodiments, the cells retain the ability to transcribe the polynucleotide of interest in the presence of the first ligand and in the absence of the second ligand, after having been frozen and thawed at least one, at least two, at least three, or at least four times.

In certain embodiments, the cell culture has a cell density of at least 400,000 to one million viable cells per ml while in a repressed state in the presence of the second ligand. While in an induced state in the presence of the first ligand and the absence of the second ligand, the cell culture can have a cell density at least 600,000 to two million viable cells per ml. In further embodiments, the cells are grown in research or production bioreactors having a volume of, for example, at least 2 liters, at least 5 liters, at least 10 liters, 50 liters, at least 75 liters, at least 100 liters, at least 150 liters, at least 200 liters, at least 500 liters, at least 1,000 liters, at least 2,000 liters, at least 5,000 liters, at least 10,000 liters, at least 15,000 liters, at least 20,000 liters or more.

In certain embodiments, the cell is a mammalian cell, such as a primate, canine or rodent cell. In a more specific embodiments, the cell is a CHO cell, such as CHO-K1 cell, a BHK cell, a Human amniotic cell or a HEK293 cell.

In certain embodiments, in the absence of a first ligand and presence of a second ligand, transcription of the polynucleotide sequence encoding the polypeptide of interest is substantially reduced. For example, at least a 10-fold decrease in transcription achieved relative to the level of transcription as seen in the presence of the first ligand and absence of the second ligand. In certain embodiments, at least a 20-fold decrease in transcription achieved relative to the level of transcription as seen in the presence of the first ligand and absence of the second ligand. In certain embodiments, at least a 50-fold decrease in transcription achieved relative to the level of transcription as seen in the presence of the first ligand and absence of the second ligand. In certain embodiments, at least a 100-fold decrease in transcription achieved relative to the level of transcription as seen in the presence of the first ligand and absence of the second ligand. In certain embodiments, at least a 500-fold decrease in transcription achieved relative to the level of transcription as seen in the presence of the first ligand and absence of the second ligand.

In certain embodiments, the degree of transcription of the polynucleotide sequence of interest achieved in the presence of the first ligand and the absence of the second ligand may be at least 10-fold greater than in the absence of the first ligand and the presence of the second ligand. In certain embodiments, the degree of transcription of the polynucleotide sequence of interest achieved in the presence of the first ligand and the absence of the second ligand may be at least 20-fold greater than in the absence of the first ligand and the presence of the second ligand. In certain embodiments, the degree of transcription of the polynucleotide sequence of interest achieved in the presence of the first ligand and the absence of the second ligand may be at least 50-fold greater than in the absence of the first ligand and the presence of the second ligand. In certain embodiments, the degree of transcription of the polynucleotide sequence of interest achieved in the presence of the first ligand and the absence of the second ligand may be at least 100-fold greater than in the absence of the first ligand and the presence of the second ligand. In certain embodiments, the degree of transcription of the polynucleotide sequence of interest achieved in the presence of the first ligand and the absence of the second ligand may be at least 500-fold greater than in the absence of the first ligand and the presence of the second ligand.

Other embodiments, aspects, objects and advantages will become apparent from a review of the ensuing detailed description.

### BRIEF DESCRIPTION OF THE FIGURES

FIG. 1 depicts induction of the transcription of a gene of interest (in this example, a polynucleotide encoding a crimson fluorescent protein) in the presence of dox and absence of OHT. The left side depicts the repressed state where dox is absent and OHT is present. The right side depicts the induced state where dox is present and OHT is absent. The figure shows an example of a tandem arrangement of a Tet Response Element (TRE) and an Arc operator (AO).

FIG. 2 depicts the results of transcription of a gene of interest (GOI) (in this example, a polynucleotide encoding a crimson fluorescent protein) in the presence or absence of the ligands. See FIG. 1. A polynucleotide encoding the crimson fluorescent protein was transcribed under the control of rtTA coupled with ArcER (TRE-AO) or under the control of CMV-TO. In the presence of E2 and the absence of Dox, very low levels of transcription of the GOI (crimson) are observed (TRE-TO repressed (+E2/-Dox) as compared to the control (Negative, unmodified cell) and CMV-TO. In the presence of Dox and absence of E2, high levels of transcription of the GOI (crimson) are observed (TRE-AO induced).

FIG. 3 depicts an optional embodiment where the expression of a regulatory fusion protein, such as rtTA, or a repressor protein can be regulated by a second RFP and associated elements, such as ArcER, AO and OHT.

FIG. 4 depicts an embodiment where polynucleotide encoding the GOI, crimson fluorescent protein, was under the control of rtTA and TetR. A Tet Response Element (TRE) and a Tet operator (TetO) are in a tandem arrangement. Induction of the transcription of a gene of interest (in this example, a polynucleotide encoding a crimson fluorescent protein) occurs in the presence of dox and absence of a ligand such as E2 or OHT, thereby allowing the rtTA fusion protein to be expressed. See FIG. 3 regarding optional regulated expression of a regulatory fusion protein, such as rtTA.

FIG. 5 depicts results of the transcription of a gene of interest (GOI) (in this example, a polynucleotide encoding a crimson fluorescent protein) in the presence or absence of the ligands. Transcription of a polynucleotide encoding the crimson fluorescent protein was under the control of rtTA and TetR (TRE-TO). See FIG. 4. In the presence of E2 and the absence of dox, very low levels of transcription of the GOI (crimson) are observed (TRE-TO repressed (+E2/-Dox)) that are nearly identical to those of the control (Negative, unmodified cell). In the presence of dox and absence of E2, high levels of transcription of the GOI (crimson) are observed (TRE-TO induced).

FIG. 6 depicts control of transcription of a gene of interest (in this figure, a polynucleotide encoding a cytotoxic gene) in the absence of dox and presence of OHT, which provides a repressed state.

FIG. 7 depicts control of transcription of a gene of interest (in this figure, a polynucleotide encoding a cytotoxic gene) in the presence of dox and absence of OHT, which provides an induced state.

FIG. 8 depicts a western blot of proteins produced by HEK293 cells transformed with Rep78 and Rep 52 genes under tight control by TRE-AO. When the HEK293 cells were in a repressed state (-) (without Dox and with E2), Rep78 and Rep52 were not produced. In the induced state (+) (with Dox and without E2), both Rep 78 and Rep 52 are produced. The left column of the western blot has size markers.

### DETAILED DESCRIPTION OF THE INVENTIONS

Unless defined otherwise, all technical and scientific terms used herein have the same meaning as commonly understood by one of ordinary skill in the art to which this inventions belongs.

### Definitions

The term “about” in the context of numerical values and ranges refers to values or ranges that approximate or are close to the recited values or ranges such that the inventions can perform, such as having a sought rate, amount, density, degree, increase, decrease, extent of transcription or extent of polypeptide expression, concentration, or time, as is apparent from the teachings contained herein. Thus, this term encompasses values beyond those simply resulting from systematic error. For example, “about” can signify values either above or below the stated value in a range of approx. +/- 10% or more or less depending on the ability to perform.

An “effective amount” of a compound refers to the amount of compound needed to cause the intended result, and is typically defined in terms of molar or weight concentration of the compound when present in a medium. Ligands are an example of compounds.

“Capable of binding” refers to the ability of a molecule, such a regulatory fusion protein or portion thereof to bind to another molecule or portion thereof, such ligand binding domains, nucleic acid binding domains, operators, response elements and the like. Typically, binding can permit an action or function or block an action or function.

A “nucleic acid moiety” includes any arrangement of single stranded or double stranded nucleotide sequences. Nucleic acid moieties can include, but are not limited to, polynucleotides, promoters, enhancers, operators, repressors, transcription termination signals, ribosomal entry sites and polyadenylation signals.

A “DNA cassette” or “cassette” is a type of nucleic acid moiety that comprises at least a promoter, at least one open reading frame and optionally a polyadenylation signal. One or more operators also are optional. A DNA cassette thus is a polynucleotide that comprises two or more shorter polynucleotides. A cassette can comprise one or more gene and promoters, enhancers, operators, repressors, transcription termination signals, ribosomal entry sites, introns and polyadenylation signals.

“Operably linked” refers to one or more nucleotide sequences in functional relationships with one or more other nucleotide sequences. Such functional relationships can directly or indirectly control, which refers to inducing, causing, regulating, enhancing, facilitating, permitting, influencing, attenuating, stopping, preventing, repressing and/or blocking one or more actions or activities in accordance with the selected design for a selected purpose. Exemplars include single-stranded or double-stranded nucleic acid moieties, and can comprise two or more nucleotide sequences arranged within a given moiety in such a way that sequence(s) can exert at least one functional effect on other(s). For example, a promoter operably linked to the coding region of a DNA polynucleotide sequence can facilitate transcription of the coding region. Other elements, such as enhancers, operators, repressors, transcription termination signals, ribosomal entry sites and polyadenylation signals also can be operably linked with a polynucleotide of interest to control its transcription. Arrangements and spacing to achieve operable linkages can be ascertained by approaches available to the person skilled in the art, such as screening using western blots and RT-PCR.

“Operator” indicates a DNA sequence that is introduced in or near a polynucleotide sequence in such a way that the polynucleotide sequence may be regulated by the interaction of a molecule capable of binding to the operator and, as a result, prevent or allow transcription of the polynucleotide sequence, as the case may be. One skilled in the art will recognize that the operator must be located sufficiently in proximity to the promoter such that it is capable of controlling transcription by the promoter, which can be considered a type of operable linkage. The operator may be placed either downstream or upstream of the promoter. These include, but are not limited to, the operator region of the Lex A gene of E. coli, which binds the Lex A peptide and the lactose and 45 tryptophan operators, which bind the repressor proteins encoded by the Lad and trpR genes of E. coli. The bacteriophage operators from the lambda Pi and the phage P22 Mnt and Arc. In an alternative embodiment, when the transcription blocking domain of the RFP is a restriction enzyme, the operator is the recognition sequence for that enzyme. Preferred operators are the Tet operator and the Arc operator exemplified herein. Operators can have a native sequence or a mutant sequence (for example, synthetic or semi-synthetic). For example, mutant sequences of the Tet operator are disclosed in Wissmann et al., Nucleic Acids Res. 14: 4253-66 (1986). TRE also functions as an operator and can comprise native operator sequences, mutant operator sequences or combinations of native and mutant operator sequences.

The phrases “percent identity” or “% identical,” in their various grammatical forms, when describing a sequence is meant to include homologous sequences that display tile recited identity along regions of contiguous homology, but the presence of gaps, deletions, or insertions that have no homolog in the compared sequence are not taken into account in calculating percent identity. As used herein, a “percent identity” or “% identical” determination between homologs would not include a comparison of sequences where the homolog has no homologous sequence to compare in an alignment. Thus, “percent identity” and “% identical” do not include penalties for gaps, deletions, and insertions.

A “homologous sequence” in the context of nucleic acid sequences refers to a sequence that is substantially homologous to a reference nucleic acid sequence. In some embodiments, two sequences are considered to be substantially homologous if at least 50%, 55%, 60%, 65%, 70%, 75%, 80%, 85%, 90%, 91%, 92%, 93%, 94%, 95% 96%, 97%, 98%, 99% or more of their corresponding nucleotides are identical over a relevant stretch of residues. In some embodiments, the relevant stretch is a complete (i.e., full) sequence.

“Polynucleotide” includes a sequence of nucleotides covalently joined, and includes RNA and DNA. Oligonucleotides are considered shorter polynucleotides. Genes are DNA polynucleotides (polydeoxyribonucleic acid) that ultimately encode polypeptides, which are translated from RNA (polyribonucleic acid) that was typically transcribed from DNA. DNA polynucleotides also can encode RNA polynucleotides that is not translated, but rather function as RNA “products”. The type of polynucleotide (that is, DNA or RNA) is apparent from the context of the usage of the term. A polynucleotide referred to or identified by the polypeptide it encodes sets forth and covers all suitable sequences in accordance with codon degeneracy. Polynucleotides, including those disclosed herein, include percent identity sequences and homologous sequences when indicated.

“Polypeptide” includes a sequence of amino acids covalently joined. Polypeptides include natural, semi-synthetic and synthetic proteins and protein fragments. “Polypeptide” and “protein” can be used interchangeably. Oligopeptides are considered shorter polypeptides.

“Promoter” indicates a DNA sequence that cause transcription of a DNA sequence to which it is operably linked, i.e., linked in such a way as to permit transcription of the nucleotide sequence of interest when the appropriate signals are present and/or repressors are absent. The transcription of a polynucleotide of interest may be placed under control of any promoter or enhancer element known in the art. A eukaryotic promoter can be operably linked to a TATA Box, and most eukaryotic promoters have TATA boxes. The TATA Box is typically located upstream of the transcription start site.

Useful promoters that may be used include, but are not limited to, the SV40 early promoter region, SV40 E/L (early late) promoter, the promoter contained in the 3′ long terminal repeat of Rous sarcoma virus, the regulatory sequences of the metallothionein gene, mouse or human cytomegalovirus major immediate early (CMV-MIE) promoter and other CMV promoters, including CMVmin promoters. Plant expression vectors comprising the nopaline synthetase promoter region, the cauliflower mosaic virus 35S RNA promoter, and the promoter of the photosynthetic enzyme ribulose biphosphate carboxylase; promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I; insulin; immuno globulin; mouse mammary tumor virus; albumin; C.-feto protein; C.1-antitrypsin; 3-globin, and myosin light chain-2. Various forms of the CMV promoter are preferred and the CMVmin promoter is exemplified here.

Minimal promoters, such as CMVmin promoters, tend to be truncated promoters or core promoters and can be used in controlled expression systems. Minimal promoters are more amenable to control. Minimal promoters and development approaches are widely known and disclosed in, for example, Saxena et al., Methods Molec. Biol. 1651:263-73 (2017); Ede et al., ACS Synth Biol. 5:395-404 (2016); Brown et al., Biotech Bioeng. 111:1638-47 (2014); Morita et al., Biotechniques 0:1-5 (2012); Lagrange et al., Genes Dev. 12:34-44 (1998). There are many CMVmin promoters described in the field. It also is possible to use TATA box sequences to perform the role of a promoter.

“Recombinase recognition sites” (RRS), also known as heterospecific recombination sites,” are used in recombinase mediated cassette exchange (RMCE). Cre/Lox, Dre/Rox, Vre/Vlox, SCre/Slox and Flp/Frt are suitable systems, for example . Suitable RRSs for use according to the inventions include Lox P, Lox 66, Lox 71, Lox 511, Lox 2272, Lox 2372, Lox 5171, Lox M2, Lox M3, lox M7 and Lox M11. These sites can be referred to generically as first (1), second (2), third (3), fourth (4), fifth (5), sixth (6), seventh (7), eighth (8), ninth (9), tenth (10), etc., as is apparent from the context of usage.

A “regulatory fusion protein” or “RFP” is a protein that comprises a ligand binding domain and a DNA binding domain that originate from different proteins. Steroid-binding domains of the glucocorticoid or estrogen nuclear receptors can be employed as ligand binding domains. The reverse Tet DNA binding domain (rTet) also is useful as a ligand binding domain, and can bind DNA as well. Exemplary RFPs for use according to the inventions described herein are the reverse tetracycline transactivator (rtTA) and the fusion protein comprising the Arc repressor binding domain (Arc) and the estrogen receptor ligand binding domain (ArcER). Other components for RFPs include the DNA-binding domain of yeast activator GAL4 fused to HSV VP16; the KRAB domain of human Kox1 fused to a prokaryotic Tet repressor (TetR-KRAB); ligand-binding domain of the estrogen receptor (ER) to the carboxyl end of the tTA transactivator (TetR-VP16); and a catalytically inactive formof Cas9 fused to repeats of the minimal activation domain of VP16 (dCas9-VP64). Other fusion proteins include LexA-VP16 and Lacl-VP16. Polynucleotides encoding regulatory fusion proteins (for example, rtTA and ArcEr) can be integrated into the cellular genome as described herein.

A “repressor protein”, also referred to as a “repressor,” is a protein that can bind to DNA in order to repressor transcription. Repressors are of eukaryotic and prokaryotic origin. Prokaryotic repressors are preferred. Examples of repressor families include: TetR, LysR, Lacl, ArsR, IcIR, MerR, AsnC, MarR, DeoR, GntR and Crp families. Repressor proteins in the TetR family include: ArcR, ActII, AmeR, AmrR, ArpR, BpeR, EnvR, EthR, HemR, HydR, IfeR, LanK, LfrR, LmrA, MtrR, Pip, PqrA, QacR, RifQ, RmrR, SimReg2, SmeT, SrpR, TcmR, TetR, TtgR, TrgW, UrdK, VarR YdeS, ArpA, BarA, Aur1B, CalR1, CprB, FarA, JadR*, JadR2, MphB, NonG, PhIF, TyIO, VanT, TarA, TyIP, BM1P1, Bm3R1, ButR, CampR, CamR, DhaR, KstR, LexA-like, AcnR, PaaRR, Psbl, Th1R, UidR, YDH1, BetI, McbR, MphR, PhaD, Q9ZF45, TtK, Yhgd, YixD, CasR, IcaR, LitR, LuxR, LuxT, OpaR, Orf2, SmcR, HapR, Ef0113, HIyllR, BarB, ScbR, MmfR, AmtR, PsrA andYjdC proteins See Ramos et al., Microbiol. Mol. Biol. Rev., 69: 326-56 (2005). Still other repressors include PurR, LacR, MetJ and PadR, Repressor proteins are encoded by genes referred to as “repressor genes” or “repressor protein genes.”

“Reporter proteins” as used herein, refers to any protein capable of generating directly or indirectly a detectable signal. Reporter proteins typically fluoresce, or catalyze a colorimetric or fluorescent reaction, and often are referred to as “fluorescent proteins” or “color proteins.” However, a reporter protein also can be non-enzymatic and non-fluorescent as long as it can be detected by another protein or moiety, such as a cell surface protein detected with a fluorescent ligand. A reporter protein also can be an inactive protein that is made functional through interaction with another protein that is fluorescent or catalyzes a reaction. Accordingly, any suitable reporter protein, as understood by one of skill in the art, could be used. In some aspects, the reporter protein may be selected from fluorescent protein, luciferase, alkaline phosphatase, β-galactosidase, β-lactamase, dihydrofolate reductase, ubiquitin, and variants thereof. Fluorescent proteins are useful for the recognition of gene cassettes that have or have not been successfully inserted and/or replaced, as the case may be. Fluid cytometry and fluorescence-activated cell sorting are suitable for detection. Examples of fluorescent proteins are well-known in the art, including, but not limited to Discosoma coral (DsRed), green fluorescent protein (GFP), enhanced green fluorescent protein (eGFP), cyano fluorescent protein (CFP), enhanced cyano fluorescent protein (eCFP), yellow fluorescent protein (YFP), enhanced yellow fluorescent protein (eYFP) and far-red fluorescent protein (e.g. mKate, mKate2, mPlum, mRaspberry or E2-crimson. See, for example, U.S. Pat. os. 9,816,110. Reporter proteins are encoded by polynucleotides, and are referred to herein as “reporter genes” or “reporter protein genes.” Reporter genes and proteins can be referred to generically as first (1), second (2), third (3), fourth (4), fifth (5), sixth (6), seventh (7), eighth (8), ninth (9), tenth (10), etc., as is apparent from the context of usage. Reporters can be considered a type of marker. “Color” or “fluorescent,” in their various grammatical forms, also can be used the more specifically refer to a reporter protein or gene.

“Selectable” or “selection” marker proteins include proteins conferring certain traits, including but not limited to drug resistance or other selective advantages. Selection markers can give the cell receiving the selectable marker gene resistance towards a certain toxin, drug, antibiotic or other compound and permit the cell to produce protein and propagate in the presence of the toxin, drug, antibiotic or other compound, and are often referred to as “positive selectable markers.” Suitable examples of antibiotic resistance markers include, but are not limited to, proteins that impart resistance to various antibiotics, such as kanamycin, spectinomycin, neomycin, gentamycin (G418), ampicillin, tetracycline, chloramphenicol, puromycin, hygromycin, zeocin, and/or blasticidin. There are other selectable markers, often referred to as “negative selectable markers,” which cause a cell to stop propagating, stop protein production and/or are lethal to the cell in the presence of the negative selectable marker proteins. Thymidine kinase and certain fusion proteins can serve as negative selectable markers, including but not limited to GyrB-PKR. See White et al., Biotechniques, 50: 303-309 (May 2011). Selectable marker proteins and corresponding genes can be referred to generically as first (1), second (2), third (3), fourth (4), fifth (5), sixth (6), seventh (7), eighth (8), ninth (9), tenth (10), etc., as is apparent from the context of usage.

A “Stable Integration Site” or “SIS” is a region for site-specific integration of DNA polynucleotides, including cassettes that comprise genes and/or other open reading frames, promoters and optionally other elements. Stable Integration Sites can be created according to the methods of the inventions described and depicted herein. Constructs can be inserted into an SIS by a variety of approaches. Multiple Stable Integration Sites can be created and located on different chromosomes, different regions of the same chromosome or different positions in a same region of a chromosome.

A “Tetracycline Response Element” or “TRE” comprises seven copies of the 19 nucleotide TetO spaced apart by spacers comprising 17-18 nucleotides, and are commercially available. TetO sequences can vary and nucleotide substitutions are known. For example, altered sequences based on the Tet operator are disclosed in Wissmann et al., Nucleic Acids Res. 14: 4253-66 (1986). The spacers are not sequence specific. The spacers can be similar, but all should not be identical. A TRE is considered a type of operator as used herein.

All numerical limits and ranges set forth herein include all numbers or values thereabout or there between of the numbers of the range or limit. The ranges and limits described herein expressly denominate and set forth all integers, decimals and fractional values defined and encompassed by the range or limit. The ranges and limits described herein expressly denominate and set forth all integers, decimals and fractional values defined and encompassed by the range or limit. Thus, a recitation of ranges of values herein are merely intended to serve as a shorthand method of referring individually to each separate value falling within the range, unless otherwise indicated herein, and each separate value is incorporated into the specification as if it were individually recited herein.

### Detailed Description

The present inventions generally relate to constructs that allow for the tight control of transcription of a polynucleotide sequence in a cell. The present inventions are based on the inquiry and determination that stable cells can be established that transcribe polynucleotide sequences where transcription of the polynucleotide sequence is controlled by an controllable expression system. Cells expressing polynucleotide sequences where transcription of the polynucleotide sequence of interest is controlled by the controllable expression system described herein can be used in a wide variety of applications. The polynucleotide sequence of interest can encode a polypeptide of interest or a product of interest. The controllable expression system described herein is especially useful for controlling the expression of polypeptides of interest and/or products of interest that are toxic or inhibitory to the host cell.

The cells described herein provide the specific advantages that the cells are stable. By “stable” it is meant that the cell can be used to establish a cell line that has regions of interest that are functionally homogenous in culture. The regions of interest would include, for example, polynucleotides of interest and associated promoters, operators, internal ribosome entry sites (IRES), polyadenylation signals and non-translated RNAs, which can be monitored.

Additionally, cells described herein provide the additional advantage that the transcription of polynucleotide encoding the polypeptide of interest is tightly controlled so that the cells are able to survive to a stage permitting large scale expression of the polypeptide of interest. By “tightly controlled” it is meant that in the absence of a first ligand (alternatively in the presence of the first ligand as well) and presence of a second ligand, transcription of the polynucleotide sequence encoding the polypeptide of interest is substantially reduced. Tightest control is achieved in the absence of the first ligand and the presence of the second ligand. For example, in certain embodiments in the repressed state at least a 10-fold decrease in transcription achieved relative to the level of transcription as seen in the presence of the first ligand and absence of the second ligand in the induced state. In certain embodiments in the repressed state, at least a 20-fold decrease in transcription achieved relative to the level of transcription as seen in the presence of the first ligand and absence of the second ligand in the induced state. In certain embodiments in the repressed state, at least a 50-fold decrease in transcription achieved relative to the level of transcription as seen in the presence of the first ligand and absence of the second ligand in the induced state. In certain embodiments in the repressed state, at least a 100-fold decrease in transcription achieved relative to the level of transcription as seen in the presence of the first ligand and absence of the second ligand in the induced state. In certain embodiments in the repressed state, at least a 500-fold decrease in transcription achieved relative to the level of transcription as seen in the presence of the first ligand and absence of the second ligand in the induced state.

As a corollary to the repressed state, the degree of induction of transcription of the polynucleotide sequence of interest seen in the presence of the first ligand and the absence of the second ligand may be at least 10-fold greater in certain embodiments than in the repressed state. In certain embodiments, the degree of induction of transcription of the polynucleotide sequence of interest in seen in the presence of the first ligand and the absence of the second ligand may be at least 20-fold greater than in the repressed state. In certain embodiments, the degree of induction of transcription of the polynucleotide sequence of interest in seen in the presence of the first ligand and the absence of the second ligand may be at least 50-fold greater than in the repressed state. In certain embodiments, the degree of induction of transcription of the polynucleotide sequence of interest in seen in the presence of the first ligand and the absence of the second ligand may be at least 100-fold greater than in the repressed state. In certain embodiments, the degree of induction of transcription of the polynucleotide sequence of interest in seen in the presence of the first ligand and the absence of the second ligand may be at least 500-fold greater than in the repressed state.

The degree or amount of transcription of the polynucleotide sequence interest may be determined by methods known to those of skill in the art. For example, the level of expression of a polypeptide of interest in a host cell can be determined based on the amount of the corresponding mRNA that is present in the cell. Messenger RNA transcribed from a polynucleotide sequence can be quantified by various methods known by those of skill in the art, including but not limited to, Northern blot hybridization, ribonuclease RNA protection, in situ hybridization to cellular RNA or by PCR.

By way of a further example, the level of expression of a polypeptide of interest in a host cell may also be determined based on the amount of polypeptide of interest encoded by the selected sequence. Polypeptides encoded by a polynucleotide sequence can be quantified by various methods known by those of skill in the art, including but not limited to, ELISA, Western blotting, radioimmunoassays, immunoprecipitation, assay of the biological activity of the polypeptide, immunostaining of the polypeptide followed by FACS analysis or by homogeneous time resolved fluorescence assays (HTRF).

### Controllable Transcription and Expression Systems

The present inventions relate to a controllable transcription and expression system that may be used to control the transcription of any polynucleotide sequence of interest. The described controllable transcription and expression system comprises at least two controllable operator systems. One of the operator systems can be located 5′ to a promoter that is operably linked to the polynucleotide sequence of interest and the second operator system can be located 3′ of the promoter. The operator systems may comprise operators that are operably linked to a promoter that drives transcription of the polynucleotide sequence of interest. The polynucleotide of interest may encode a polypeptide and/or product (for example, RNA) of interest.

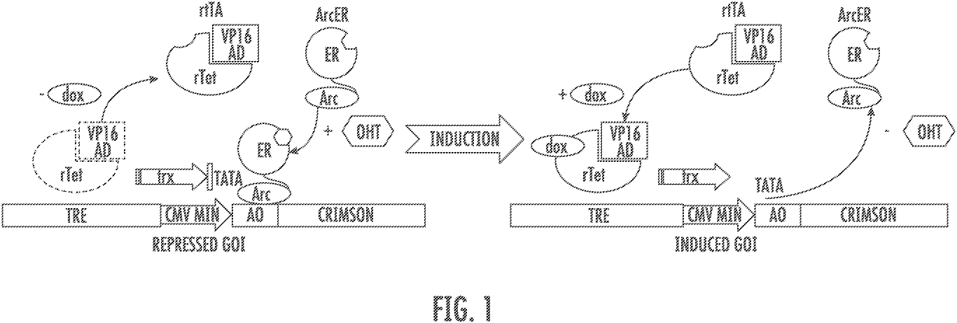

Controllable transcription as described herein allows for transcription of the polynucleotide of interest in the presence of a first ligand and the absence of a second ligand. Briefly, when present, the first ligand binds to a ligand binding site on a first regulatory fusion protein (RFP) which comprises a (1) a transcription activating domain fused to a DNA binding domain; and (2) a ligand-binding domain. Upon binding of the first ligand to the ligand-binding domain of the first RFP, the DNA binding domain of the first RFP binds to a first operator, allowing for transcription from the promoter, but only if transcription is not inhibited by the second operator system. The second operator system is controlled by a second ligand. Briefly, when present, the second ligand binds to a ligand binding site on a second regulatory fusion protein (RFP) which comprises a (1) a transcription blocking domain fused to a DNA binding domain; and (2) a ligand-binding domain. Upon binding of the second ligand to the ligand-binding domain of the second RFP, the DNA binding domain of the second RFP binds to a second operator, blocking transcription from the promoter. Thus, in the presence of the second ligand and absence of the first ligand, transcription is repressed; whereas in the absence of the second ligand and the presence of the first ligand, transcription is permitted. FIGS. 1, 4, 6 and 7 illustrate examples of control of transcription utilizing this system.

The first operator system may comprise at least one operator that is operably linked to a promoter that drives transcription of the polynucleotide sequence of interest. The operator of the first operator system may be located 5′ to a promoter that is operably linked to the polynucleotide sequence of interest. Examples of such configurations are shown in FIGS. 1, 4, 6 and 7, where the first operator system comprises a TRE.

The first operator system also may comprise a regulatory fusion protein (RFP) which comprises a (1) a transcription activating domain fused to a DNA binding domain; and (2) a ligand-binding domain. The first operator system may further comprise a ligand that binds to the ligand-binding domain of the first RFP. Upon binding of the ligand to the ligand-binding domain of the first RFP, the DNA binding domain of the RFP binds to the operator, for example TRE, thereby allowing for transcription from the promoter, but only if transcription is not inhibited by the second operator system as discussed herein. Other system components are known to those of skill in the art, and include for example, the tetracycline on systems (Tet-On), tetracycline on advanced (Tet-On Advanced), tetracycline on 3G systems (Tet-On 3G), cumate-inducible systems, lactose-inducible systems, and variations thereof.

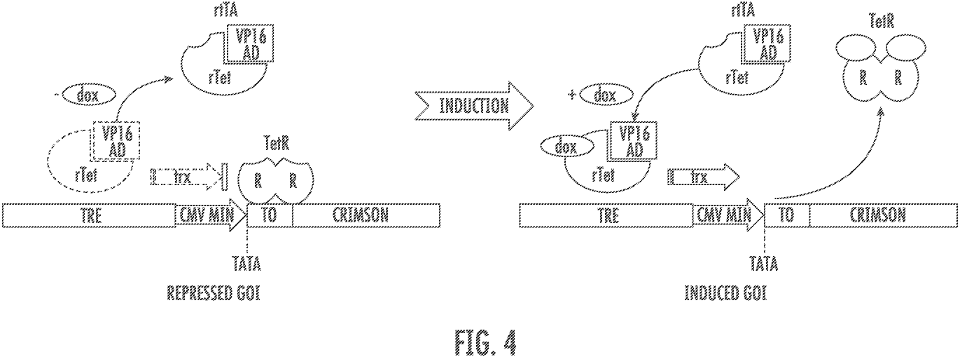

The second operator system can be located 3′ to a promoter that is operably linked to the polynucleotide sequence encoding the polypeptide of interest. Examples of such configurations are shown in FIGS. 1, 4, 6 and 7. The second regulatory element comprises at least one operator that is operably linked to a promoter that drives transcription of the polynucleotide sequence of interest. Examples of such configurations are shown in FIGS. 1, 4, 6 and 7, where the second operator system comprises either a tetracycline operator (TetO) or an Arc operator (ArcO). In some embodiments, the second operator is TetO (FIG. 4). In some embodiments, the second operator is ArcO (FIGS. 1, 6 and 7).

By way of a non-limiting example, suitable controllable operator components for the second operator system are described in U.S. Pat. o. 9,469,856.

Suitable promoters for use with the described system are known and can be determined by those of skill in the art in combinations of choice. In some embodiments, the promoter operably linked to the polynucleotide sequences may be selected from, but is not limited to, the SV40 early promoter region, SV40 E/L promoter, the promoter contained in the 3′ long terminal repeat of Rous sarcoma virus, the regulatory sequences of the metallothionein gene, mouse or human cytomegalovirus major immediate early (MIE) promoter; CMVmin promoters, plant expression vectors comprising the nopaline synthetase promoter region, the cauliflower mosaic virus 35S RNA promoter, and the promoter of the photosynthetic enzyme ribulose biphosphate carboxylase; promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I; insulin; immunoglobulin; mouse mammary tumor virus; albumin; α-fetoprotein; α1-antitrypsin; β-globin; and myosin light chain-2. In some embodiments, the promoter is the human CMV-MIEmin or other CMVmin promoters. Approaches for developing minimal promoters are described in Saxena et al., Methods Molec. Biol. 1651:263-73 (2017); Ede et al., ACS Synth Biol. 5:395-404 (2016); Brown et al., Biotech Bioeng. 111:1638-47 (2014); Morita et al., Biotechniques 0:1-5 (2012); Lagrange et al. Genes Dev. 12:34-44 (1998).

In some embodiments, the polynucleotide sequence of interest encodes a polypeptide of interest. The polynucleotide of interest can be a native gene, including variants thereof, or a synthetic, semi-synthetic or optimized sequence. In other embodiments, the polynucleotide sequence of interest encodes a product (for example, RNA) of interest. More specifically, products of interest may be non-coding RNAs.

“Protein of interest” or “polypeptide of interest” (POI) can have any amino acid sequence, and includes any protein, polypeptide, or peptide, and derivatives, components, domains, chains and fragments thereof. Included are, but not limited to, viral proteins, bacterial proteins, fungal proteins, plant proteins and animal (including human) proteins. Protein types can include, but are not limited to, antibodies, bi-specific antibodies, multi-specific antibodies, antibody chains (including heavy and light), antibody fragments, Fv fragments, Fc fragments, Fc-containing proteins, Fc-fusion proteins, receptor Fc-fusion proteins, receptors, receptor domains, trap and mini-trap proteins, enzymes, factors, repressors, activators, ligands, reporter proteins, selection proteins, protein hormones, protein toxins, structural proteins, storage proteins, transport proteins, neurotransmitters and contractile proteins. Derivatives, components, chains and fragments of the above also are included. The sequences can be natural, semi-synthetic or synthetic. Proteins of interest and polypeptides of interest are encoded by “genes of interest,” which also can be referred to as “polynucleotides of interest.” Where multiple genes (same or different) are integrated, they can be referred to as “first,” “second”, “third,” “fourth,” “fifth,” “sixth,” “seventh,” “eighth,” “ninth,” “tenth,” etc. as is apparent from the context of use.

A polypeptide of interest also can include cytotoxic proteins, such as viral proteins. For example, adenovirus E1A, E1B, E2A and E4 are used to perform functions for production of adeno-associated virus (AAV), but have been reported to be toxic effects in certain cell types. AAV Rep also has been reported to by cytotoxic in certain cell types. Additionally, proteins used in genetic alterations, such as Cre recombinase, Flp recombinase, Zinc finger (ZFN) proteins and dimers, TALEN, bxb 1 integrase, CRISPR associated proteins (Types I-VI; including Cas1, Cas2, Cas3, Cas4, Cas, Cas6, Cas7, Cas8, Cas9, Cas10, Cas11, Cas12 and Cas13) and other nucleases and integrases, can be POIs, and thereby controlled according to the present inventions.

### Cells Capable of Controlled Transcription and Expression

In one aspect, a cell comprising a promoter operably linked to a polynucleotide sequence of interest, wherein the promoter is controlled by at least two operators operably linked to the promoter is provided. The promoter operably linked to the polynucleotide sequence of interest, the operators operably linked to the promoter may be integrated into the cell genome. Transcription of the polynucleotide sequence of interest is controlled by the operators, allowing the transcription of the polynucleotide of interest to be permitted or repressed as preferred.

Cells that are suitable for use with these inventions can be readily selected by those of skill in the art. In some embodiments the cell line is a eukaryotic cell line such as a yeast cell line, insect cell line (for example, Sf9 and Sf21 cells) or a mammalian cell line. Preferred mammalian cells include primate cells (including human), canine cells and rodent cells. Cells can be primary cells or immortalized cells. Suitable cells can be selected from Vero cells, COS cells, HEK293 cells, HeLa cells, CHO cells, BHK cells, Sp2/0 cells, MDCK cells, amniotic cells (including human), embryonic cells, cell lines transfected with viral genes, for example, AD5 E1, including but not limited to an immortalized human retinal cell transfected with an adenovirus gene, for example, a PER.C6 cell, or an NSO cell. In some embodiments, the cell is a Chinese hamster ovary (CHO) cell line. Some examples of CHO cells include, but are not limited to, CHO-ori, CHO-K1, CHO-s, CHO-DHB11, CHO-DXB11, CHO-K1 SV, and mutants/variants thereof. In further preferred embodiments, the CHO cell may be the CHO cell line designated K1. Examples of HEK293 cells include, but are not limited, to HEK293, HEK293A, HEK293E, HEK293F, HEK293FT, HEK293FTM, HEK293H, HEK293MSR, HEK293S, HEK293SG, HEK293SGGD, HEK293T and mutants and variants thereof.

An illustration of the constructs used in creating a cell expressing polynucleotide sequences encoding the crimson fluorescent protein is shown in FIG. 1. The construct comprises an expression cassette comprising polynucleotide sequences encoding a gene of interest (for example, crimson), at least one promoter, and at least two operators. In brief, as shown in FIG. 1, transcription of the polynucleotide sequences encoding crimson from the CMVmin promoter is controlled by the tetracycline operator (TetO) and the Arc operator (AO or ArcO).

In some embodiments, the cell further comprises a polynucleotide sequence encoding one or more regulatory fusion proteins (RFP). The RFP may comprise (a) a transcription activating domain fused to a DNA binding domain and (b) a ligand-binding domain. The ligand is capable of binding to the ligand-binding domain the RFP.

In some embodiments, the cell further comprises a polynucleotide sequence encoding one or more regulatory fusion proteins (RFP), regulatory proteins or repressor proteins. The RFP may comprise (a) a transcription blocking domain fused to a DNA binding domain and (b) a ligand-binding domain. The transcription blocking domain may comprise an Arc repressor DNA-binding domain. A regulatory protein may be TetR. Transcription inhibition of the polynucleotide of interest by binding to the Tet or Arc operator. In some embodiments, the operator is Tet and the transcription blocking domain is a Tet repressor. In other embodiments, the operator is Arc and the transcription blocking domain is an Arc repressor DNA-binding domain.

The cells may further comprise elements that regulate the transcription of the polynucleotide sequence(s) encoding one or more regulatory fusion proteins (RFP). By way of a non-limiting example, transcription of the polynucleotide sequence(s) encoding one or more regulatory fusion proteins (RFP) may be controlled by a Tet-On system, such that the polynucleotide sequence(s) encoding one or more regulatory fusion proteins (RFP) is only transcribed in the presence of Dox. In another embodiment, transcription of rtTA (an RFP) optionally can be under the control of ArcER and AO.

In some instances, the polynucleotide sequence of interest, as well as the operably linked promoter and operators, may be introduced into the cell by transfection of a plasmid containing said polynucleotide sequences and elements. Accordingly, the inventions include the generation of cells as described and cells comprising a plasmid construct as described.

Suitable plasmid constructs can be made by those of skill in the art. Useful regulatory elements, described previously or known in the art, can also be included in the plasmid constructs used to transfect the cells. Some non-limiting examples of useful regulatory elements include, but are not limited to, promoters, enhancers, sequences encoding suitable mRNA ribosomal binding sites, and sequences that control the termination of transcription and translation. Suitable plasmid constructs also may comprise non-transcribed elements such as an origin of replication, other 5′ or 3′ flanking non-transcribed sequences, and 5′ or 3′ non-translated sequences such as splice donor and acceptor sites. One or more markers genes may also be incorporated. Useful markers for use in the present inventions are known and can be readily identified by those of skill in the art.

A plasmid construct encoding a gene of interest may be delivered to the cell using a viral vector or via a non-viral method of transfer.

Non-viral methods of nucleic acid transfer include naked nucleic acid, liposomes, and protein/nucleic acid conjugates. A plasmid construct that is introduced to the cell may be linear or circular, may be single-stranded or doublestranded, and may be DNA, RNA, or any modification or combination thereof.

A plasmid construct may be introduced into the cell by transfection. Those of skill in the art are aware of numerous different transfection protocols, and can select an appropriate system for use in transfecting cells. Generally, transfection methods include, but are not limited to, viral transduction, cationic transfection, liposome transfection, dendrimer transfection, electroporation, heat shock, nucleofection transfection, magnetofection, nanoparticles, biolistic particle delivery (gene gun), and proprietary transfection reagents such as Lipofectamine, Dojindo Hilymax, Fugene, jetPEI, Effectene, or DreamFect.

Upon introduction into the cell, some polynucleotide sequences from the plasmid construct may be integrated into the cell genome, such as a chromosome. In some instances, integration of the polynucleotide sequence into a genome may be achieved with lox sites.

In an embodiment, the promoter operably linked to the polynucleotide sequences of interest, the first operator operably linked to the promoter, and the second operator, are integrated into the cell genome. Other polynucleotides, such as those encoding regulatory fusion proteins (for example, rtTA and ArcEr) and repressor proteins (for example TetR), also can be integrated into the cellular genome as described herein.

In some embodiments, the genomic integration is random. Methods of achieving random genomic integration are known by those of skill in the art, and suitable means can be identified by those of skill. For example, a linearized plasmid with a selectable marker can be used for genomic integration in random locations.

In some embodiments, the genomic integration is site-specific. Site-specific integration refers to the integration at a specific site within a chromosome. Methods of achieving site-specific integration are known by those of skill in the art, and suitable approaches can be identified by those of skill. By way of a non-limiting example, one approach for site-specific integration in CHO cells is described in U.S. Pat. o. 7,771,997 (“Stable Site 1”), which is hereby incorporated by reference, including sequence information. U.S. Patent No. 7,771,997 describes integration sites located at enhanced expression and stability regions. Another suitable integration site is described in U.S. Pat. o. 9,816,110 (“Stable Site 2”), which is hereby incorporated by reference, including sequence information. Regeneron provides a suite of goods and services referred to as EESYR®. CHO cells with integrated sequences in Stable Site 1 and Stable Site 2 are disclosed in US 2019/0233544 A1, which is hereby incorporated by reference, including sequence information. Sequences set forth in these patents and Examples 6 and 7 can be used according to the inventions described and depicted herein. Additionally, an AAVS1-like region and the COSMC locus in hamster cells can be used according to the inventions.

For human cells, such as HEK293 cells, integration can be achieved using the adeno-associated virus integration site 1 (AAVS1) via appropriate plasmids. See Lou et al., Human Gene Therapy Methods, 28: 124-38 (2017); Liu et al., BMC Research Note, 7:626 (2014). AAVS1 is reported to be located in chromosome 19. Additional sites include CCR5 and the Rosa26.

Modification of cellular genomes can be undertaken with known approaches, such as Cre/Lox, Flp/Frt, transcription activator-like effector nuclease (TALEN), a TAL effector domain fusion protein, zinc finger nuclease (ZFN), a ZFN dimer, or a RNA-guided DNA endonuclease system, such as CRISPR/Cas9. See U.S. Patent No. 9,816,110 at cols. 17-18; Sajgo et al., PLoS ONE 9: e91435 (2014); Suzuki et al., Nucl. Acids. Res. 39: e49 (2011). Modification using Bxb1 integrase in human, mouse and rat cells also can be undertaken. Russell et al., Biotechniques 40: 460-64 (2006).

To maximize stability and efficiency and facilitate integration and control of the inventions, Stable Integration Sites (SIS) can be created using Genomic Safe Harbors and the like in a wide variety of cell types and lines according to the teachings of U.S. Serial No. 63/256,675. The descriptions (including examples) and figures providing methods and cells resulting from the methods of U.S. Serial No. 63/256,675 are hereby incorporated by reference.

Accordingly, site-specific and random integration approaches can be employed in in cell. Finally, polynucleotides can exist integrated into non-chromosomal locations as known by the person skilled in the art, such as episomes.

In some embodiments, the cells described herein may comprise a polynucleotide sequence encoding a marker. In an embodiment, the polynucleotide sequence encoding the marker is linked to the polynucleotide sequences encoding the polynucleotide of interest. The polynucleotide sequence encoding the marker may be linked to the polynucleotide sequences of interest such that if the polynucleotide sequence of interest are integrated, the marker polynucleotide sequence is integrated as well. Useful markers for use in the present inventions are known and can be readily identified by those of skill in the art and include, but are not limited to, selectable markers (such as drug resistance markers) and reporter proteins, such as colorimetric/ fluorescent markers.

### Methods of Controlling Expression of Polypeptides

In another aspect, the inventions provide methods of controlling transcription of a gene of interest in a cell as described. This method has utility in a wide variety of applications, including, by way of not limiting examples, production of proteins/products of interest for therapeutic purposes.

The inventions provide methods of controlling expression of a polypeptide of interest in a cell. In some embodiments, the polypeptide of interest is toxic or inhibitory to the cell, such as a viral gene.

Production of a protein or product of interest starts with a working cell bank (WCB), which typically is stored frozen. The WCB will contain the cells in the absence or presence of the first ligand (for example, dox) and the presence of the second ligand (for example, OHT), as determined by the skilled person. The WCB is thawed and used to create the seed culture (also known as a “seed train”). At the seed culture stage, the cells can be expanded in the absence or presence of the first ligand and in the absence of the second ligand, as determined by the skilled person. As the effectiveness of the inhibition of transcription by the second ligand decreases, typically about 4 to 14 days post seeding (although the time course can be modified by, for example, adding additional second ligand to the cell culture media to delay loss of effectiveness). Following the seed culture stage, production of the protein or product of interest can commence. Non-limiting examples of embodiments are discussed below to further illustrate aspects of the inventions.

Methods and compositions are provided to facilitate the control of transcription of a polynucleotide sequence of interest in a cell culture at various scales (for example, bench top to bioreactor). Further, methods and compositions for achieving a delayed transcription of a polynucleotide of interest in cells in culture also are provided that rely on transitioning from presence of the second ligand to the presence of the first ligand and absence of the second ligand. A delayed transcription includes transcription of the polynucleotide of interest only after or at a point in time after which the cells have grown to a desired density. In general, under some circumstances, it is desirable to allow the cells to reach a desired density, for example, about 10, 20, 30, 40, 50, 60, 70, 80, 90, or 100% (that is, about 10 to 100%) of maximal achievable density in culture, before a desired amount of transcription of the polynucleotide sequence of interest occurs. In some embodiments, a cell density of about 90 to about 100% is desirable prior to full transcription of the polynucleotide sequence of interest.

In some embodiments, a cell density of at least 400,000 viable cells per ml is desirable prior to full transcription of the polynucleotide sequence of interest. In some embodiments, a cell density of at least 500,000 viable cells per ml is desirable prior to full transcription of the polynucleotide sequence of interest. In some embodiments, a cell density of at least 600,000 viable cells per ml is desirable prior to full transcription of the polynucleotide sequence of interest. In some embodiments, a cell density of at least 700,000 viable cells per ml is desirable prior to full transcription of the polynucleotide sequence of interest. In some embodiments, a cell density of at least 800,000 viable cells per ml is desirable prior to full transcription of the polynucleotide sequence of interest. In some embodiments, a cell density of at least 900,000 viable cells per ml is desirable prior to full transcription of the polynucleotide sequence of interest. In some embodiments, a cell density of at least one million viable cells per ml is desirable prior to full transcription of the polynucleotide sequence of interest. In some embodiments, a cell density of at least two million viable cells per ml is desirable prior to full transcription of the polynucleotide sequence of interest. In some embodiments, a cell density of at least three million viable cells per ml is desirable prior to full transcription of the polynucleotide sequence of interest. In some embodiments, a cell density of at least four million viable cells per ml is desirable prior to full transcription of the polynucleotide sequence of interest. In some embodiments, a cell density of at least five million viable cells per ml is desirable prior to full transcription of the polynucleotide sequence of interest. In some embodiments, a cell density of at least six million viable cells per ml is desirable prior to full transcription of the polynucleotide sequence of interest. In some embodiments, a cell density of at least seven million viable cells per ml is desirable prior to full transcription of the polynucleotide sequence of interest. In some embodiments, a cell density of at least eight million viable cells per ml is desirable prior to full transcription of the polynucleotide sequence of interest. In some embodiments, a cell density of at least nine million viable cells per ml is desirable prior to full transcription of the polynucleotide sequence of interest. In some embodiments, a cell density of at least ten million viable cells per ml is desirable prior to full transcription of the polynucleotide sequence of interest. In some embodiments, a cell density of at least twelve million viable cells per ml is desirable prior to full transcription of the polynucleotide sequence of interest. In some embodiments, a cell density of at least fifteen million viable cells per ml is desirable prior to full transcription of the polynucleotide sequence of interest. Typical ranges can be 400,000 to 3 million. Other acceptable ranges include 1 million to 15 million, 2 million to 12 million, 3 million to 10 million, 4 million to 9 million, 5 million to 8 million, and 6 million to 7 million, and any subrange within any of these ranges, as is apparent to the person skilled in the art in view of these teachings.

Delayed induction of transcription can be achieved by any suitable methodology described herein. In various embodiments, delayed induction can be achieved by growing cells to a desired cell density in the presence of an effective amount of the second ligand. In view of the teachings contained herein, the time course of inhibition can be defined by selection of the time course for maintenance of an effective amount of the second ligand in the cell culture. The removal of the second ligand can be achieved by, for example, (i) separating the cells from media containing the second ligand, (ii) diluting the cell culture with media that does not contain the second ligand, and/or (iii) splitting a mixture of the cells and media such that the second ligand is then present at a level below the effective amount (for example, an amount of the second ligand that does not substantially inhibit, or fails to inhibit, transcription of the polynucleotide sequence of interest), sometimes referred to as clearing. In various embodiments, cells are initially grown and/or stored (for example, in a working cell bank (WCB)) with an effective amount of the second ligand.

In various embodiments, cell cultures can be expanded in a medium absent an effective amount of a second ligand. More particularly, during expansion the cell culture has an effective amount of the first ligand and does not have an effective amount of the second ligand for a time period sufficient to allow the second ligand to clear, usually about 4 to 14 days, depending on the second ligand and culture conditions.

In other embodiments, once the desired culture size is reached, and the second ligand no longer effectively inhibits transcription of the cell culture, the polynucleotide of interest can be transcribed in the presence of an effective amount of the first ligand. An effective amount of the first ligand can be added to the cell culture at any appropriate time. For example, the effective amount of the first ligand can be added during seeding. In another example, the effective amount of the first ligand can be added at a later point in time, such as when the second ligand is clearing or already cleared.

One of skill in the art will be able to determine the suitable concentration of the first and second ligands to achieve effective amounts throughout the process. Exemplary concentrations of a ligand may include about 100 nM to about 1,000 nM, about 100 to 900 nM, 100 to 800 nM, about 100 nM to 700 nM, 100 nM to 600 nM, 100 nM to 500 nM, 100 nM to 400 nM, 100 nM to 300 nM, in a specific embodiment about 200 nM to about 500 nM, in another specific embodiment, about 400 nM. Other concentrations can be used as well.

All methods described herein can be performed in any suitable order unless otherwise indicated herein or otherwise clearly contradicted by context. The use of any and all examples, or exemplary language (for example, “such as”) provided herein, is intended merely to better illuminate the inventions and does not pose a limitation on the scope of the inventions unless otherwise claimed. No language in the specification should be construed as indicating any non-claimed element as essential to the practice of the inventions. As various changes could be made in the above described compositions and methods without departing from the scope of the inventions, it is intended that all matter contained in the above description and in the examples given below, shall be interpreted as illustrative and not in a limiting sense.

### EXAMPLES

The following examples are put forth so as to provide those of ordinary skill in the art how to make and use the cells, methods and compositions described herein, and are not intended to limit the scope of what the inventors regard as their inventions.

### Example 1

### Two Regulatory Fusion Proteins (rtTA and ArcER) Allow for Tight Control

CHO-K1 cells were constructed that stably express the crimson florescent protein under the control of both the TRE and ArcO. As shown in FIG. 1, transcription of crimson is from the CMVmin promoter with a TATA box. The CMVmin promoter is flanked 5′ by a TRE and 3′ by an ArcO (AO). The reverse tetracycline transcriptional activator (rtTA) is an RFP composed of a reverse Tet DNA binding domain (rTet) and the VP16 transactivation domain (VP16 AD). The rTet moiety can bind the ligand tetracycline, doxycycline (dox) and derivatives thereof. ArcER is an RFP where the Arc repressor binding domain (Arc) is fused to the estrogen receptor ligand binding domain (ER). The ER moiety can bind estrogen, estradiol (E2), tamoxifen, 4-hydroxytamoxifen (OHT) and other derivatives thereof.

FIG. 1 depicts a repressed gene of interest and induced gene of interest. In the repressed state, the first ligand, here dox, is absent, which means that rtTA is unable to bind TRE to allow transcription (trx) to proceed. Additionally, the second ligand, here OHT, is present and binds to the ArcER regulatory fusion protein (RFP), thereby resulting in inhibition of transcription of crimson.

In the induced state, the first ligand, here dox, binds to rtTA and enables it to bind TRE, which is permissive for transcription (trx). When the second ligand is absent, here OHT, ArcER does not inhibit transcription of crimson. It is believed that ArcER is not transported into the nucleus in the absence of an estrogen receptor ligand like OHT or E2. Thus, transcription can proceed where the first ligand is present and the second ligand is absent.

For example, crimson expressing CHO-K1 cells (TRE-AO) can be made by inserting a DNA cassette encoding selectable markers and reporter proteins, as well as polynucleotides encoding rtTA and ArcER into the genome of the cell at the Stable Site 1 according to U.S. Pat.No. 7,771,997. A cassette containing a polynucleotide encoding crimson with a CMVmin promoter can be inserted into the cellular genome at Site 2 according to the teachings of U.S. Pat. No. 9,816,110, for example. The reporter protein was included to confirm integration of the expression cassette into the cellular genome.

Stable integration of the two expression cassettes was confirmed utilizing the included selectable markers.

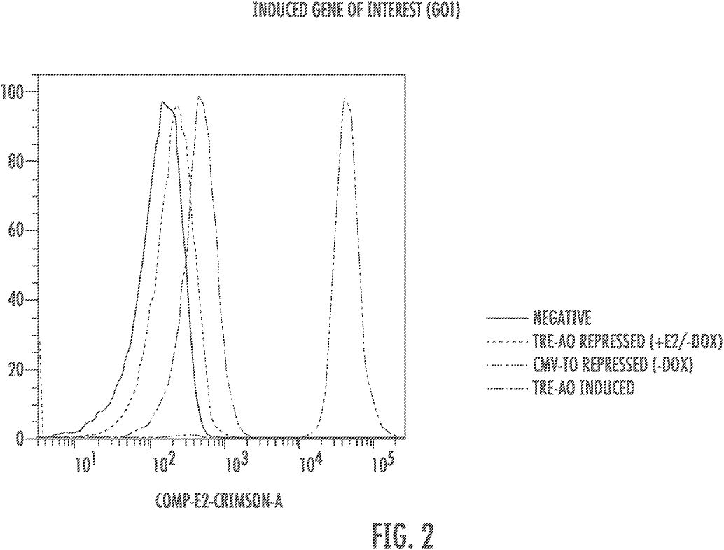

The ability to tightly control transcription of crimson in the TRE-AO CHO-K1 cells was then tested and compared to a negative control (unmodified cell). As expected, when the ligand for ArcER, here E2, was present and the ligand for rtTA, here Dox, was absent, expression of crimson was highly suppressed. As shown in FIG. 2 the levels of crimson expression were close to those of the negative control. Additionally, levels of crimson expression in the TRE-AO CHO-K1 cells were lower than those in a control cell (standard CMV-TO) that was able to express crimson in the presence of Dox. This demonstrates that the TRE-AO system provided tighter control of transcription as compared to a TetO alone. Further, with +dox and -E2, high levels of crimson expression were observed (FIG. 2, TRE-AO induced).

Thus, the TRE-AO system provides a means of tightly controlling the transcription of polynucleotides of interest.

### Example 2

### Control of the Production of Regulatory Fusion Protein or a Repressor Protein

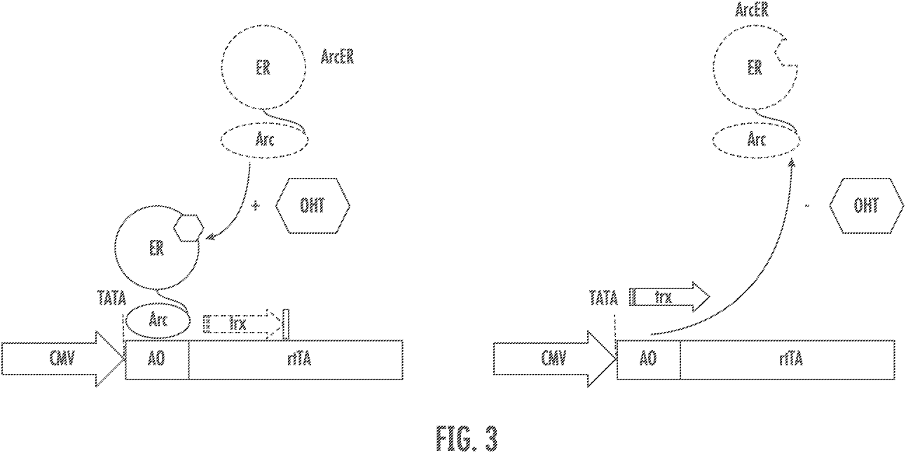

Optionally, production of a regulatory fusion protein, such as rtTA, or a repressor protein can be controlled by another regulatory fusion protein and ligand, such as ArcER and AO as depicted in FIG. 3. An exemplary construct has a CMV promoter and a TATA box and AO downstream followed by the gene encoding rtTA. ArcER in the presence of the ligand OHT can bind to AO and block transcription (trx). In the absence of a ligand like OHT, ArcER is no longer available to bind AO, which is permissive for transcription (trx) of the gene encoding rtTA.

The use of an RFP, such as ArcER, to control the level of expression of another RFP, such as rtTA, is another optional approach for controlling transcription of a polynucleotide of interest that is under control of that RFP (rtTA in this instance) according the inventions described herein.

### Example 3

### Regulatory Fusion Protein (rtTA) With Repressor Protein (TetR) Allows for Tight Control

CHO- K1 cells were constructed that stably express the crimson fluorescent protein under the control of TRE and a separate TetO. As shown in FIG. 4, transcription of crimson is from the CMVmin promoter with a TATA box. The CMVmin promoter is flanked 5′ by a TRE and 3′ by a TetO (TO). In the absence of the ligand dox, rtTA cannot bind to TRE, which prevents transcription. Additionally, the Tet Repressor protein (TetR) binds to the tetracycline operator (TO) in the absence of the ligand dox, which also blocks transcription.

When the TetR ligand is present, here dox, it binds to the rtTA, and thereby is permissive for transcription. Additionally, the dox ligand binds to TetR, which lessens the affinity of the Tet repressor for TO and is permissive for transcription. The polynucleotide encoding the repressor protein, such as TetR, can be inserted randomly into the genome or site-specifically into the genome.

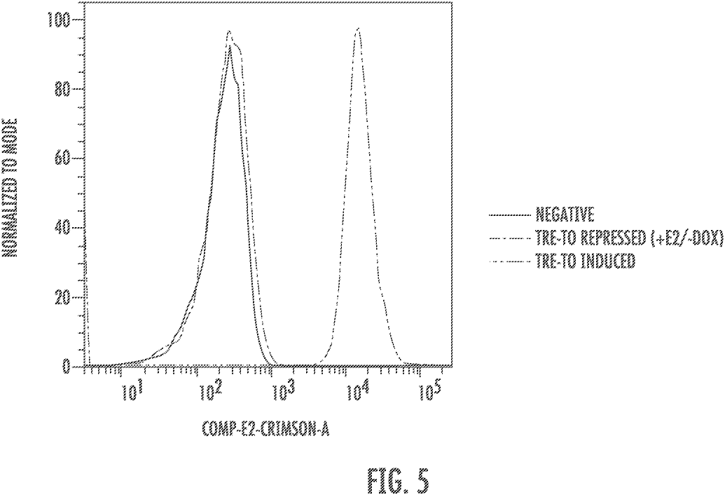

As shown in FIG. 5, transcription of a polynucleotide encoding the crimson fluorescent protein was under the control of rtTA and TetR (TRE-TO). See FIG. 4. In the presence of E2 and the absence of dox, very low levels of transcription of the GOI (crimson) are observed (TRE-TO repressed (+E2/-Dox)) that are nearly identical to those of the control (Negative, unmodified cell). In the presence of dox and absence of E2, high levels of transcription of the GOI (crimson) are observed (TRE-TO induced).

### Example 4

### Two Regulatory Fusion Proteins (rtTA and ArcER) Allow for Tight Control of a Cytotoxic Gene

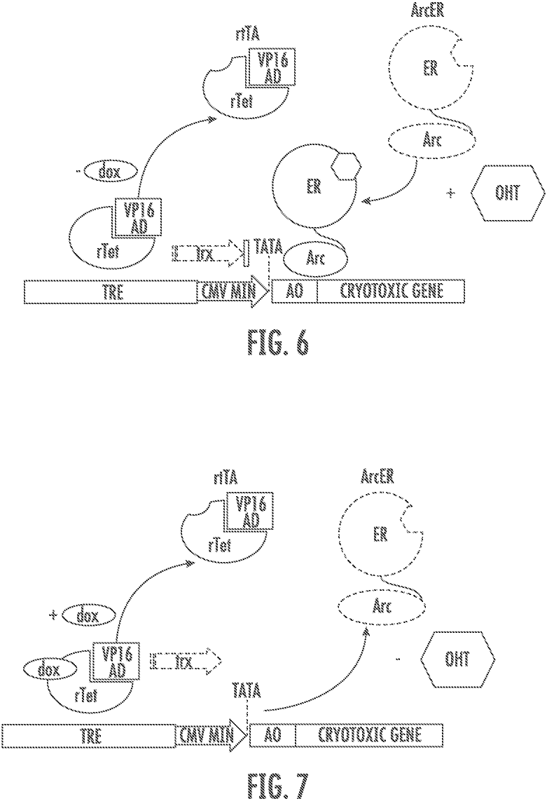

FIGS. 6 and 7 depict the rtTA coupled with ArcER to provide tight control over the transcription of a cytotoxic gene. FIG. 6 depicts a repressed stated where dox (a type of first ligand) is absent (-dox) and OHT (a type of second ligand) is present (+OHT). The absence of dox means the rtTA cannot bind to TRE to allow for the commencement of transcription. The presence of OHT allows ArcEr to bind to AO, which blocks transcription. The presence of OHT also prevents transcription of the rtTA polynucleotide where the embodiment of Example 2 and FIG. 3 is employed.

FIG. 7 depicts an induced state. Dox is present (+dox), which allows rtTA to bind to TRE and commence transcription (trx). OHT is absent (-OHT), which results in ArcER no longer being able to bind AO, which means that ArcER does not block transcription of the cytotoxic gene, and in certain embodiments allows for the transcription of rtTA, as described in Example 2 and depicted in FIG. 3.

### Example 5

### Toxic AAV Rep Genes Can Be Regulated Under Tight Control and Expressed

A HEK293 cell line was constructed with TRE and AO according the teachings contained herein controlling genes for AAV Rep78 and Rep52. See Examples 1 and 4 and FIGS. 1, 6 and 7. The embodiment shown in FIGS. 6 and 7 replaces a color gene of FIG. 1 with a cytotoxin-encoding gene, such as Rep genes. AAV Rep 78 and a truncated version, Rep 52, and both know to be toxic to human cells. Schmidt et al, J. Virol. 9441-50 (2000).

In a repressed state, rtTA is without its ligand (for example, dox) and ArcER is in the presence of its ligand (for example, OHT). See FIG. 6. In an induced state, rtTA is in the presence of its ligand, and ArcEr is in the absence of its ligand, which allows transcription (trx) of the cytotoxic gene to proceed. See FIG. 7.

In this example, the HEK293 cells were transformed with Rep78 and Rep 52, and both genes were under control of the TRE-AO system. The ligands employed were dox and E2 (instead of OHT).

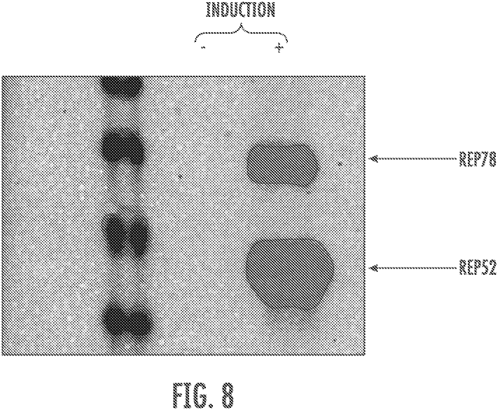

FIG. 8 discloses the results. When the HEK293 cells were in a repressed state (-), Rep78 and Rep52 were not produced. In the induced state (+), both Rep 78 and Rep 52 are produced.

### Example 6

Exemplary sequences are provided below, and other sequences (including homologs and variants) are available to the person skill in the art.

### Nucleic Acid and Amino Acid Sequences

### rtTA Nucleotide Sequence

(SEQ ID NO: 1)

ATGTCTAGACTGGATAAGTCTAAGGTGATCAATGGAGCTCTGGAACTGCT
GAATGGAGTGGGAATCGAAGGACTGACAACAAGAAAGCTGGCTCAGAAGC
TGGGAGTGGAACAGCCTACACTGTATTGGCATGTGAAGAATAAGAGAGCT
CTGCTGGATGCTCTGCCTATCGAAATGCTGGATAGACATCATACACATTT
TTGTCCTCTGGAAGGAGAATCTTGGCAGGATTTTCTGAGAAATAATGCTA
AGTCTTTTAGATGTGCTCTGCTGTCTCATAGAGATGGAGCTAAGGTGCAT
CTGGGAACAAGACCTACAGAAAAGCAGTATGAAACACTGGAAAATCAGCT
GGCTTTTCTGTGTCAGCAGGGATTTTCTCTGGAAAATGCTCTGTATGCTC
TGTCTGCTGTGGGACATTTTACACTGGGATGTGTGCTGGAAGAACAGGAA
CATCAGGTGGCTAAGGAAGAAAGAGAAACACCTACAACAGATTCTATGCC
TCCTCTGCTGAGACAGGCTATCGAACTGTTTGATAGACAGGGAGCTGAAC
CTGCTTTTCTGTTTGGACTGGAACTGATCATCTGTGGACTGGAAAAGCAG
CTGAAGTGTGAATCTGGATCTGCTTATTCTAGAGCTAGAACAAAGAATAA
TTATGGATCTACAATCGAAGGACTGCTGGATCTGCCTGATGATGATGCTC
CTGAAGAAGCTGGACTGGCTGCTCCTAGACTGTCTTTTCTGCCTGCTGGA
CATACAAGAAGACTGTCTACAGCTCCTCCTACAGATGTGTCTCTGGGAGA
TGAACTGCATCTGGATGGAGAAGATGTGGCTATGGCTCATGCTGATGCTC
TGGATGATTTTGATCTGGATATGCTGGGAGATGGAGATTCTCCTGGACCT
GGATTTACACCTCATGATTCTGCTCCTTATGGAGCTCTGGATATGGCTGA
TTTTGAATTTGAACAGATGTTTACAGATGCTCTGGGAATCGATGAATATG
GAGGATAA

### rtTA Amino Acid Sequence

(SEQ ID NO: 2)

MSRLDKSKVINGALELLNGVGIEGLTTRKLAQKLGVEQPTLYWHVKNKRA
LLDALPIEMLDRHHTHFCPLEGESWQDFLRNNAKSFRCALLSHRDGAKVH
LGTRPTEKQYETLENQLAFLCQQGFSLENALYALSAVGHFTLGCVLEEQE
HQVAKEERETPTTDSMPPLLRQAIELFDRQGAEPAFLFGLELIICGLEKQ
LKCESGSAYSRARTKNNYGSTIEGLLDLPDDDAPEEAGLAAPRLSFLPAG
HTRRLSTAPPTDVSLGDELHLDGEDVAMAHADALDDFDLDMLGDGDSPGP
GFTPHDSAPYGALDMADFEFEQMFTDALGIDEYGG*

### ArcER Nucleotide Sequence

(SEQ ID NO: 3)

ATGAAGGGCATGTCCAAGATGCCTCAGTTCAACCTGCGCTGGCCTCGCGA
GGTGCTGGACCTGGTGCGCAAGGTGGCCGAGGAGAACGGCCGCTCCGTGA
ACTCCGAAATCTACCAGCGCGTGATGGAGTCCTTCAAGAAGGAGGGCCGC
ATCGGAGCCGGAGGTGGCTCCGGAGGTGGCACCGGTGGAGGCTCTGGAGG
AGGCATGAAAGGAATGTCTAAAATGCCCCAATTTAATCTCCGGTGGCCCC
GCGAAGTCCTCGATCTCGTGCGGAAAGTCGCTGAAGAAAATGGACGGTCT
GTCAATTCTGAAATTTATCAACGGGTCATGGAATCTTTTAAAAAAGAAGG
ACGGATTGGAGCTGCTTATTCTGGATCCCGGGAATTAATTCGGCTTTCTG
CTGGAGACATGAGAGCTGCCAACCTTTGGCCAAGCCCGCTCATGATCAAA
CGCTCTAAGAAGAACAGCCTGGCCTTGTCCCTGACGGCCGACCAGATGGT
CAGTGCCTTGTTGGATGCTGAGCCCCCCATACTCTATTCCGAGTATGATC
CTACCAGACCCTTCAGTGAAGCTTCGATGATGGGCTTACTGACCAACCTG
GCAGACAGGGAGCTGGTTCACATGATCAACTGGGCGAAGAGGGTGCCAGG
CTTTGTGGATTTGACCCTCCATGATCAGGTCCACCTTCTAGAATGTGCCT
GGCTAGAGATCCTGATGATTGGTCTCGTCTGGCGCTCCATGGAGCACCCA
GTGAAGCTACTGTTTGCTCCTAACTTGCTCTTGGACAGGAACCAGGGAAA
ATGTGTAGAGGGCATGGTGGAGATCTTCGACATGCTGCTGGCTACATCAT
CTCGGTTCCGCATGATGAATCTGCAGGGAGAGGAGTTTGTGTGCCTCAAA
TCTATTATTTTGCTTAATTCTGGAGTGTACACATTTCTGTCCAGCACCCT
GAAGTCTCTGGAAGAGAAGGACCATATCCACCGAGTCCTGGACAAGATCA
CAGACACTTTGATCCACCTGATGGCCAAGGCAGGCCTGACCCTGCAGCAG
CAGCACCAGCGGCTGGCCCAGCTCCTCCTCATCCTCTCCCACATCAGGCA
CATGAGTAACAAAGGCATGGAGCATCTGTACAGCATGAAGTGCAAGAACG
TGGTGCCCCTCTATGACCTGCTGCTGGAGGCGGCGGACGCCCACCGCCTA
CATGCGCCCACTAGCCGTGGAGGGGCATCCGTGGAGGAGACGGACCAAAG
CCACTTGGCCACTGCGGGCTCTACTTCATCGCATTCCTTGCAAAAGTATT
ACATCACGGGGGAGGCAGAGGGTTTCCCTGCCACAGTCTGA

### ArcER Amino Acid Sequence

(SEQ ID NO: 4)

MKGMSKMPQFNLRWPREVLDLVRKVAEENGRSVNSEIYQRVMESFKKEGR
IGAGGGSGGGTGGGSGGGMKGMSKMPQFNLRWPREVLDLVRKVAEENGRS
VNSEIYQRVMESFKKEGRIGAAYSGSRELIRLSAGDMRAANLWPSPLMIK
RSKKNSLALSLTADQMVSALLDAEPPILYSEYDPTRPFSEASMMGLLTNL
ADRELVHMINWAKRVPGFVDLTLHDQVHLLECAWLEILMIGLVWRSMEHP
VKLLFAPNLLLDRNQGKCVEGMVEIFDMLLATSSRFRMMNLQGEEFVCLK
SIILLNSGVYTFLSSTLKSLEEKDHIHRVLDKITDTLIHLMAKAGLTLQQ
QHQRLAQLLLILSHIRHMSNKGMEHLYSMKCKNVVPLYDLLLEAADAHRL
HAPTSRGGASVEETDQSHLATAGSTSSHSLQKYYITGEAEGFPATV*

### TetR Nucleotide Sequence

(SEQ ID NO: 5)

ATGTCTAGATTAGATAAAAGTAAAGTGATTAACAGCGCATTAGAGCTGCT
TAATGAGGTCGGAATCGAAGGTTTAACAACCCGTAAACTCGCCCAGAAGC
TAGGTGTAGAGCAGCCTACATTGTATTGGCATGTAAAAAATAAGCGGGCT
TTGCTCGACGCCTTAGCCATTGAGATGTTAGATAGGCACCATACTCACTT
TTGCCCTTTAGAAGGGGAAAGCTGGCAAGATTTTTTACGTAATAACGCTA
AAAGTTTTAGATGTGCTTTACTAAGTCATCGCGATGGAGCAAAAGTACAT
TTAGGTACACGGCCTACAGAAAAACAGTATGAAACTCTCGAAAATCAATT
AGCCTTTTTATGCCAACAAGGTTTTTCACTAGAGAATGCATTATATGCAC
TCAGCGCTGTGGGGCATTTTACTTTAGGTTGCGTATTGGAAGATCAAGAG
CATCAAGTCGCTAAAGAAGAAAGGGAAACACCTACTACTGATAGTATGCC
GCCATTATTACGACAAGCTATCGAATTATTTGATCACCAAGGTGCAGAGC
CAGCCTTCTTATTCGGCCTTGAATTGATCATATGCGGATTAGAAAAACAA
CTTAAATGTGAAAGTGGGTCCGCGTACAGCGGATCCCGGGAATTCAGATC
TTATTAA

### TetR Amino Acid Sequence

(SEQ ID NO: 6)

MSRLDKSKVINSALELLNEVGIEGLTTRKLAQKLGVEQPTLYWHVKNKRA
LLDALAIEMLDRHHTHFCPLEGESWQDFLRNNAKSFRCALLSHRDGAKVH
LGTRPTEKQYETLENQLAFLCQQGFSLENALYALSAVGHFTLGCVLEDQE
HQVAKEERETPTTDSMPPLLRQAIELFDHQGAEPAFLFGLELIICGLEKQ
LKCESGSAYSGSREFRSY

### Tet Operator

(SEQ ID NO: 7)

TCCCTATCAGTGATAGAGA

### Tet Response Element

(SEQ ID NO: 8)

TCCCTATCAGTGATAGAGAACGTATGAAGAGTTTACTCCCTATCAGTGAT
AGAGAACGTATGCAGACTTTACTCCCTATCAGTGATAGGGAACGTATAAG
GAGTTTACTCCCTATCAGTGATAGAGAACGTATGACCAGTTTACTCCCTA
TCAGTGATAGAGAACGTATCTACAGTTTACTCCCTATCAGTGATAGAGAA
CGTATATCCAGTTTACTCCCTATCAGTGATAGAGA

### Arc Operator

(SEQ ID NO: 9)

ATGATAGAAGCACTCTACTATTC

### hCMVmin Promoter

(SEQ ID NO: 10)

GCGTATAAGCTTTAGGCGTGTACGGTGGGAGGCCTATATAAGCAGAGCTC

### CHO and Mouse Stable Site 1 Sequences - U.S. Patent No. 7,771,997

211> 6473

<212> DNA

<213> Cricetulus griseus

<400> 1 (SEQ ID NO: 11)

tctagaaaca aaaccaaaaa tattaagtca ggcttggctt caggtgctgg ggtggagtgc 60
tgacaaaaat acacaaattc ctggctttct aaggcttttt cggggattca ggtattgggt 120
gatggtagaa taaaaatctg aaacataggt gatgtatctg ccatactgca tgggtgtgta 180
tgtgtgtgta tgtgtgtctg tgtgtgtgcc cagacagaaa taccatgaag gaaaaaaaca 240
cttcaaagac aggagagaag agtgacctgg gaaggactcc ccaatgagat gagaactgag 300
cacatgccag aggaggtgag gactgaacca ttcaacacaa gtggtgaata gtcctgcaga 360
cacagagagg gccagaagca ctcagaactc cagggggtca ggagtggttc tctggaggct 420
tctgcccttg gaggttcctg aggaggaggc ttccatattg aaaatgtagt tagtggccgt 480
ttccattagt acagtgacta gagagagctg agggaccact ggactgaggc ctagatgctc 540
agtcagatgg ccatgaaagc ctagacaagc acttccgggt ggaaaggaaa cagcaggtgt 600
gaggggtcag gggcaagtta gtgggagagg tcttccagat gaagtagcag gaacggagac 660
gcactggatg gccccacttg tcaaccagca aaagcttgga tcttgttcta agaggccagg 720
gacatgacaa gggtgatctc ggtttttaaa aggctttgtg ttacctaatc acttctatta 780
gtcagatact ttgtaacaca aatgagtact tggcctgtat tttagaaact tctgggatcc 840
tgaaaaaaca caatgacatt ctggctgcaa cacctggaga ctcccagcca ggccctggac 900
ccgggtccat tcatgcaaat actcagggac agattcttca ctaggtactg atgagctgtc 960
ttggatgcaa atgtggcctc ttcattttac tacaagtcac catgagtcag gaggtgctgt 1020
ttgcacagtg tgactaagtg atggagtgtt gactgcagcc attcccggcc ccagcttgtg 1080
agagagatcc ttttaaattg aaagtaagct caaagttacc acgaagccac acatgtataa 1140
actgtgtgaa taatctgtgc acatacacaa accatgtgaa taatctgtgt acatgtataa 1200
actgtgtgaa taatctgtgt gcagcctttc cttacctact accttccagt gatcaggttt 1260
ggactgcctg tgtgctactg gaccctgaat gtccccaccg ctgtcccctg tcttttacga 1320
ttctgacatt tttaataaat tcagcggctt cccctctgct ctgtgcctag ctataccttg 1380
gtactctgca ttttggtttc tgtgacattt ctctgtgact ctgctacatt ctcagatgac 1440
atgtgacaca gaaggtgttc cctctggaga catgtgatgt ccctgtcatt agtggaatca 1500
gatgccccca aactgttgtc cagtgtttgg gaaagtgaca cgtgaaggag gatcaggaaa 1560
agaggggtgg aaatcaagat gtgtctgagt atctcatgtc cctgagtggt ccaggctgct 1620
gacttcactc ccccaagtga gggaggccat ggtgagtaca cacacctcac acatactata 1680
tccaacacac acacacacac acacacacac acgcacgcac gcacgcacgc acgcacacat 1740
gcacacacac gaactacatt tcacaaacca catacgcata ttacacccca aacgtatcac 1800
ctatacatac cacacataca cacccctcca cacatcacac acataccaca cccacacaca 1860
gcacacacat acataggcac acattcacac accacacata tacatttgtg tatgcataca 1920
tgcatacaca cacaggcaca cagacaccac acacatgcat tgtgtacgca cacatgcata 1980
cacacacata ggcacacatt gagcacacac atacatttgt gtacgcacac tacatagaca 2040
tatatgcatt tgtatatgca cacatgcatg cacacataca taggcacaca tagagcacac 2100
acatacattt gtgtatgcac acatgcacac accaatcaca tgggaagact caggttcttc 2160
actaaggttc acatgaactt agcagttcct ggttatctcg tgaaacttgg aagattgctg 2220
tggagaagag gaagcgttgg cttgagccct ggcagcaatt aaccccgccc agaagaagta 2280
ggtttaaaaa tgagagggtc tcaatgtgga acccgcaggg cgccagttca gagaagagac 2340
ctacccaagc caactgagag caaaggcaga gggatgaacc tgggatgtag tttgaacctc 2400
tgtaccagct gggcttcatg ctattttgtt atatctttat taaatattct tttagtttta 2460
tgtgcgtgaa taccttgctt gcataaatgt atgggcactg tatgtgttct tggtgccggt 2520
ggaggccagg agagggcatg gatcctccgg agctggcgtt tgagacagtt gtgacccaca 2580
gtgtggggtc tgggaactgg gtcttagtgt tccgcaagtg cagctggggc tcttaacctc 2640
tgagccatcc ctccagcttc aagaaactta ttttcttagg acatggggga agggatccag 2700
ggctttaggc ttgtttgttc agcaaatact cttttcgtgt attttgaatt ttattttatt 2760
ttactttttt gggatagaat cacattctgc agctcaggct gggcctgaac tcatcaaaat 2820
cctcctgtct cagtctacca ggtgataaga ttactgatgt gagcctggct ttgacaagca 2880
ctttagagtc cccagccctt ctggacactt gttccaagta taatatatat atatatatat 2940
atatatatat atatatatat atatattgtg tgtgtgtgtt tgtgtgtgta tgagacactt 3000
gctctaaggg tatcatatat atccttgatt tgcttttaat ttatttttta attaaaaatg 3060
attagctaca tgtcacctgt atgcgtctgt atcatctata tatccttcct tccttctctc 3120
tctttctctc ttcttcttct cacccccaag catctatttt caaatccttg tgccgaggag 3180
atgccaagag tctcgttggg ggagatggtg agggggcgat acaggggaag agcaggagga 3240
aagggggaca gactggtgtg ggtctttgga gagctcagga gaatagcagc gatcttccct 3300
gtccctggtg tcacctctta cagccaacac cattttgtgg cctggcagaa gagttgtcaa 3360
gctggtcgca ggtctgccac acaaccccaa tctggcccca agaaaaggca cctgtgtgtg 3420
actctggggt taaaggcgct gcctggtcgt ctccagctgg acttgaaact cccgtttaat 3480
aaagagttct gcaaaataat acccgcagag tcacagtgcc aggttcccgt gctttcctga 3540
agcgccaggc acgggttccc taggaaatgg ggccttgctt gccaagctcc cacggcttgc 3600
cctgcaaacg gcctgaatga tctggcactc tgcgttgcca ctgggatgaa atggaaaaaa 3660
gaaaaagaag aagtgtctct ggaagcgggc gcgctcacac aaacccgcaa cgattgtgta 3720
aacactctcc attgagaatc tggagtgcgg ttgccctcta ctggggagct gaagacagct 3780
agtgggggcg gggggaggac cgtgctagca tccttccacg gtgctcgctg gctgtggtgc 3840
atgccgggaa ccgaaacgcg gaactaaagt caagtcttgc tttggtggaa ctgacaatca 3900
acgaaatcac ttcgattgtt ttcctctttt tactggaatt cttggatttg atagatgggg 3960
gaggatcaga gggggagggg aggggcgggg agacggaggg aggaggggag gaggggagga 4020
ggggaggagg ggaggagggg aagggatgga ggaaaatact aacttttcta attcaacatg 4080
acaaagattc ggagaaagtg caccgctagt gaccgggagg aggaatgccc tattgggcat 4140
tatattccct gtcgtctaat ggaatcaaac tcttggttcc agcaccaagg attctgagcc 4200
tatcctattc aagacagtaa ctacagccca cacggaagag gctatacaac tgaagaaata 4260
aaattttcac tttatttcat ttctgtgact gcatgttcac atgtagagag ccacctgtgt 4320
ctaggggctg atgtgctggg cagtagagtt ctgagcccgt taactggaac aacccagaac 4380
tcccaccaca gttagagctt gctgagagag ggaggccctt ggtgagattt ctttgtgtat 4440
ttatttagag acagggtctc atactgtagt ccaagctagc ctccagctca cagaaattct 4500
cctgttccgg tttccaaagt actggagtta tgagtgtgtg ttaattgaac gctaagaatt 4560
tgctgattga agaaaacctc aagtgggttt ggctaatccc cacgacccca gaggctgagg 4620
caggaggaat gagagaattc aaggtttgcc agagccacag ggtgagctca atgtggagac 4680
tgtgagggtg agctcaatgt ggagactgtg agggtgagct caatgtggag actgtgaggg 4740
tgagctcaat gtggagactg tgagggtgag ctcaatgtgg agactgtgag ggtgagctca 4800
atgtggagac ctgtatcaag ataataatag tagtagtaac aatgcaggcg agggtgtggt 4860
tgagtggtag agcagttagt tgatttgaca tgcttgaggt ctcccggtcc atctgtggcc 4920
ctgcaacagg aagggaggga ggaagggggg gaacgagaga gaggaaagag agacagaagc 4980
taagataggg aatgagagag gaaggaagaa acgggaagaa attcagactc cttcctgagt 5040
tccgccaacg cctagtgaca tcctgtgcac accctaaggt ggcctttgtg tggcactggc 5100
ttgggtggtc gggaaaggca ttttcagctt gttgcagaac tgccacagta gcatgctggg 5160
tccgtgaaag tttctgcccg ttaacaagaa gtctctacta cttgtgacct caccagtgaa 5220
aatttcttta attgtctcct ggtgttctgg gttttgcatt tttgtttcta aggatacatt 5280
cctgggtgat gtcatgaagt ccccaaagac acagtggggc tgtgttggat tgggaaagat 5340
gatttatctg gggtgtcaaa aggaaaagaa gggaaacagg cacttgggaa aatgtcctcc 5400
cgcccacccg aattttggct tggcaaccgt ggtggaggag caagaaacac gtggacgttt 5460
gaggaggcat ggggtcctag gaggacagga agcagaagga gagagctggg ctgacagcct 5520
gcaggcattg cacagtttca gaaggagatt acagcatgac tgagttttta gggatccaac 5580
agggacctgg gtagagattc tgtgggctct gaggcaactt gacctcagcc agatggtatt 5640
tgaataacct gctcttagag ggaaaacaga catagcaaac agagccacgt ttagtgatga 5700
aactctcact ttgcctgagt catgtgcggc catgcccagg ggtcaggctg acactcaact 5760
caaaaacaag tgagaaattg aagacaatcc gtggtggcag ctactggaag ggccaccaca 5820
tccccagaaa gagtggagct gctaaaaagc catttgtgat aggcacagtt atcttgaatg 5880
catggagcag agattacgga aaaatcgaga atgttaatga ggcaacattc gagttgagtc 5940
attcagtgtg ggaaacccag acgcttccat cccctaaaag gaacatcttg ctctcagtca 6000
aaatggaaat aaaaattggg gcttgaattt ggcaaatgat tcagaactct gtgtaggtat 6060
tttcacacgc acagtggata attttcatgt tggagtttat ttgtgctaaa aggcagaaaa 6120
gggtaaaaag cacatcttaa gagttatgag gttctacgaa taaaaataat gttacttaca 6180
gctattcctt aattagtacc cccttccacc tgtggtaatt tcctgagata gtcagtgggg 6240
aaaagatctc tccttctctt ctttctcccc ctcccctcct ctccctccct ccctccctcc 6300
ctccctcctc tccctccctc cccctttcct tctttctttg ctccttctcc tctgcctcct 6360
tctccctttc ttcttcattt attctaagta gcttttaaca gcacaccaat tacctgtgta 6420
taacgggaaa acacaggctc aagcagctta gagaagattg atctgtgttc act 6473

<211> 7045

<212> DNA

<213> Cricetulus griseus

<400> 2 (SEQ ID NO: 12)

actagcgtgc aattcagagg tgggtgaaga taaaaggcaa acatttgagg ccatttcctt 60
atttggcacg gcacttagga agtggaacat gcctaatcta ctggtttgta ccacctttcc 120
ctataatgga ctgtttggga agctcctggg caaccgattc tggcatctca ttggtcagag 180
gcctgttaaa tggtactctt atttgcaaag aaggctgtaa cttgtagctt taaaagcctc 240
tcctcaagaa agaagggaga aaggatatgg ctagacatat ctaatagact taaccactgt 300
gaaaagcctt agtatgaatc agatagaacc tatttttaac tcagttttga aaaaaataat 360
ctttatattt atttgtgtgt gtgtgtgtgt gtgtgtgtgt gtgtgtgtgt gtgtgtgtgt 420
gaaccacatg tagcaggtgc tggaggaggc cagaagaggg caccagatct cctggaactg 480
acaccacaca tggttatgag ctgcctgatg tgggtgctgg gaactgaact ctcgtgttct 540
gcaagagcag caactgttct cttaactgat gagccatctc tccagccccc cccataattt 600
taattgttca ttttagtaaa ttttattcat aatcaattat cacagtataa aacaatgatt 660
ttatatatat catatacata tcaaggatga cagtgagggg gatatgtgtg tgtgtgtgtg 720
tgtgtgtgtg tgtgtgtgtg tgtgttattt gtgtgtgtgc tttttaagaa ggtgccatag 780
tcactgcatt tctctgaagg atttcaaagg aatgagacat gtctgtctgc caggaaccct 840
atcttcctct ttgggaatct gacccaaatg aggtattctg aggaactgaa tgaagagctc 900
aagtagcagt gtcttaaacc caaatgtgct gtctagagaa agtcaacgtc atcagtgagc 960
tgaggagaga tttactgagc ggaagacaag cgctctttga tttaagtggc tcgaacagtc 1020
acggctgtgg agtggagcct gtgctcaggt ctgaggcagt ctttgctagc cagctgtgat 1080
gagcagtgaa gaaagggtgg agatggaggc agggtgggag cagggctatg gttcagacta 1140
ggtatcgtga gcacaccagc tggttgactt gtggtctgtg ggtcaggcgt tgtaaacgcc 1200
ctcagggtca ggcagtcaca ttgcttgaag ctgaatgggt gaggcaacac agagagtgca 1260
aagaaggcaa agtaccacct cttccccgac ccaggtcact tctgggttat agctgagact 1320
ccggacagca tgcaaccagc tggttagagc ttcagggaaa acttgatgtc tgcatgttgc 1380
tatgaaatgt gattcggtac atctggagaa aatttataat gctggctcag tcaagcactg 1440
aacaaaggta ccttggcttt gggagctaca tgacattgac ttgtaggcag actttttttt 1500
ttctgcccgc caattcccag ataaccaata tggaggctca atattaatta taaatgctcg 1560
gctgatagct caggcttgtt actagctaac tcttccaact taaatgaacc catttctatt 1620
atctacattc tgccacgtga ctttaccttg tacttcctgt ttcctctcct tgtctgactc 1680
tgcccttctg cttcccagag tccttagtct ggttctcctg cctaacctta tcctgcccag 1740
ctgctgacca agcatttata attaatatta agtctcccag tgagactctc atccagggag 1800
gacttgggtg ctcccccctc ctcattgcca tccgtgtctt cctcttccct cgcttccccc 1860
tcctcttcct gctcttcctc ctccacccct cctttcatag tattgatggc aagggtgttc 1920
tagaatggag gagtgcccat aggcatgcaa agaaaccagt taggatgctc tgtgaggggt 1980
tgtaatcata agcgatggac acaattcaag ccacagagtg aagacggaag gatgcactgt 2040
gctctagagc aacttctggg gcagaatcac agggtgagtt tctgacttga gggcgaagag 2100
gccacgagga agggagtgag tttgtctgag ctagaagcta cggcccacct cttggtagca 2160
gacctgccca caagcatgct ttgttaatca tgtgggatct gattttcctc taaatctatg 2220
ttcaactctt aagaaaatgt gaattctcac attaaaattt agatatacgt cttttggtgg 2280
ggggggtgta aaaaatcctc aagaatatgg atttctgggg gccggagaga tggctcagag 2340
gttaagagaa ctggttgctc ttctagacat tctgagttca attcccagca accacatggt 2400
ggctcacaac catctgtaat gcgacctggt gccatcttct gacatgcatg gatacatgca 2460
ggcagaaagc tgtatacata gtaaattgat aaatcttttt ttaaaaagag tatggattct 2520
gccgggtgtt ggtggcgcac gcctttaatc ccagcactct ggaggcagag gcaggtggat 2580
ctctgtgagt tcgagaccag cctggtctat aagagctagt tccaggacag cctccaaagc 2640
cacagagaaa ccctgtctcg aaaaaccaaa aaaaaaaaaa aaaaaaaaaa aaaaaaaaga 2700
gtatggattc taagaaagcc gtaacagctg gagctgtgta cggagttcag cgtggtacta 2760
gaagaacaga cattcatgat gaaacacccc aggattttta cttagtatct agtttccatt 2820
gttgttttga gaccggctct tatgctctcc aggctggcct caaactgctg atcttcccgc 2880
ctctacctct caagtcctgg gactacttgg ctcataaaac agtttttgtc gggctccctg 2940
aagttatggt tgtacaaacc gtgggggtca atatactcac ttgggcagag agagaaggtc 3000
tgaatcccag acaatgactg catctcagga cagttgggaa gaggacaatg gcagaaggac 3060
ttagaaaaga tagactggag ggtggaaaag cagcaggaac agagaaacaa aacaggaagc 3120
ttgctatcca gggccactct ggagtcctgt ggcaagatgg aagcgggcta ggggaataca 3180
tttgtgctac tgtgtgtgtg tgtgtgtgtg tgtgtgtgtg tgtgtgtgat caatgcctat 3240
caatgttgaa ggggaaatat gtataccaca ttgattctgg gagcaattct cagtatctgg 3300
cctagagaaa ggaatggccc ctgcagaata gacagagtga atggtgccct ttatcatttg 3360
ctaaagtgaa ggagaaataa acatccttcc atagagtttc aggtaaatga accccacagt 3420
tcatctgtgc cgtggtggag gcctggccaa cagttaaaaa gattagacac ggacaaagtc 3480
tgaaggaaac acctcgaata ggaagaggag agccacctca ttctgtaact ttcctcaagg 3540
ggaagatgtt ccaagagtgg gaataaatgg tcaaaggggg gatttttaat taggaaaacg 3600
atttcctgta tcacttgtga aactggaggt tgatttgggg cataggacaa tagatttgat 3660
gctttgcaaa aagctgtttc aaagcagaga aatggaatag agacaattat gtagcgagga 3720
gggagggtgg ggcgaagatg gagacagaga agtggaagct gactttaggg aagaggaaca 3780
tagaccacag gggcggggcg gggggcaggg gcggggggcg gggctcaaag gaggcagtgg 3840
gaacgttgct agtgttcgca gcgtaagcgt gaatgtgcaa gcgtctttgt ggtgtgtgac 3900
caggagtagc gtggctggct tgtgtgctgc ttgtaatccc agtctttgag gtttccacac 3960
tgttccacag tgggtgtgat tttccctcgg agagcatgag ggctctgctt tccccacatc 4020
ctccccagcg ttcgttggta tttgtttcca agatgttagt gggtgagaca aagcctctct 4080
gttgatttgc ctttaacagg tgacaaaaaa agctcaacca ggagacattt ttgccttctt 4140
ggaaggtaat gctcccatgt agagcaatgg gacccatctc taaggtgagg ctactcttgc 4200
agtttgcacc cagctcttct gatgcaggaa ggaagttggt gggcaagcaa gactgtttgc 4260
ttcttgcgat ggacacattc tgcacacaaa ggctcaggag gggagaaggc tgtttgatgt 4320
ttagcactca ggaaggcccc tgatgcatct gtgattagct gtctccatct gtggagcaga 4380
cacggactaa ctaaaaacca gtgtttttaa attgtcaagc ctttaaggtg aggaaattga 4440
cttattgtgc tgggccatac gtagagcaag tgctctgcat tgggccaacc cccggctctg 4500
gtttctaggc accagaatgg cctagaacta actcacaatc ctcccattcc aggtctcagg 4560
tgctagaatg aaccactata ccagcctgcc tgcctgccta cctgccttcc taaattttaa 4620
atcatgggga gtaggggaga atacacttat cttagttagg gtttctattg ctgtgaagag 4680
acaccatgag catggcaact cttataaagg aaaacattta gttgggtggc agtttcagag 4740
gttttagtac attgtcatca tggctgggaa catgatggca tgcagacaga catggtgctg 4800
gagaaaggga tgagagtcct acatcttgca ggcaacagga cctcagctga gacactggct 4860
ggtaccctga gcataggaaa cctcacagcc caccctcaca gtgacatatt tccttcaaca 4920
aagccatacc tcctaatagt gccactccct atgagatgac agggccaatt acattcaaac 4980
tgctataaca ctttaaagta ttttattttt attattgtaa attatgtatg tagctgggtg 5040
gtggcagccg aggtgcacgc ctttaatccc agcacttggg aggcagaggc agatggatct 5100
ctgtgagttc aagaccagcc tggtctataa gagctagttg caaggaagga tatacaaaga 5160
acagttctag gatagccttc aaagccacag agaagtgctg tcttgaaaac caaaaattgt 5220
gctgggacct gtctctgctt tggttgcttc ccactccccc agagctggac tcttggtcaa 5280
cactgaatca gctgcaaaat aaactcctgg attcctctct tgtaacagga gcccgaagtc 5340
aggcgcccac ttgtcttctc gcaggattgc catagacttt ttctgtgtgc ccaccattcc 5400
agactgaagt agagatggca gtggcagaga ctgggaaggc tgcaacgaaa acaggaagtt 5460
attgcaccct gggaatagtc tggaaatgaa gcttcaaaac ttgcttcatg ttcagttgta 5520
cacagactca ctcccaggtt gactcacacg tgtaaatatt cctgactatg tctgcactgc 5580
ttttatctga tgcttccttc ccaaaatgcc aagtgtacaa ggtgagggaa tcacccttgg 5640
attcagagcc cagggtcgtc ctccttaacc tggacttgtc tttctccggc agcctctgac 5700
acccctcccc ccattttctc tatcagaagg tctgagcaga gttggggcac gctcatgtcc 5760
tgatacactc cttgtcttcc tgaagatcta acttctgacc cagaaagatg gctaaggtgg 5820
tgaagtgttt gacatgaaga cttggtctta agaactggag caggggaaaa aagtcggatg 5880
tggcagcatg tacccgaaat cccagaactg gggaggtaga gacggatgag tgcccggggc 5940
tagctggctg ctcagccagc ctagctgaat tgccaaattc caactcctat tgaaaaacct 6000
ttaccaaaca aacaaacaaa caaataataa caacaacaac aacaacaaac taccccatac 6060
aaggtgggcg gctcttggct cttgaggaat gactcaccca aacccaaagc ttgccacagc 6120
tgttctctgg cctaaatggg gtgggggtgg ggcagagaca gagacagaga gagacatgac 6180
ttcctgggct gggctgtgtg ctctaggcca ccaggaactt tcctgtcttg ctctctgtct 6240
ggcacagcca gagcaccagc acccagcagg tgcacacacc tccctccgtg cttcttgagc 6300
aaacacaggt gccttggtct gtctattgaa ccggagtaag ttcttgcaga tgtatgcatg 6360
gaaacaacat tgtcctggtt ttatttctac tgttgtgata aaaaccgggg aactccagga 6420
agcagctgag gcagaggcaa atgcaaggaa tgctgcctcc tagcttgctc cccatggctt 6480
gccgggcctg ctttctgcaa gcccttctct ccccattggc atgcctgaca tgaacagcgt 6540
ttgaaatgct ctcaaatgtc actttcaaag aaggcttctc tgatcttgct aactaaatca 6600
gaccatgttt caccgtgcat tatctttctg ctgtctgtct gtctgtctgt ctgtctatct 6660
gtctatcatc tatcaatcat ctatctatct atcttctatt tatctaccta tcattcaatc 6720
atctatcttc taactagtta tcatttattt atttgtttac ttactttttt tatttgagac 6780
agtatttctc tgagtgacag ccttggctgt cctggaaccc attctgtaac caggctgtcc 6840
tcaaactcac agagatccaa ctgcctctgc ctctctggtg ctggggttaa agacgtgcac 6900
caccaacgcc ccgctctatc atctatttat gtacttatta ttcagtcatt atctatcctc 6960
taactatcca tcatctgtct atccatcatc tatctatcta tctatctatc tatctatcta 7020
tctatcatcc atctataatc aattg 7045

<211> 6473

<212> DNA

<213> Cricetulus griseus

<400> 3 (SEQ ID NO: 13)

agtgaacaca gatcaatctt ctctaagctg cttgagcctg tgttttcccg ttatacacag 60
gtaattggtg tgctgttaaa agctacttag aataaatgaa gaagaaaggg agaaggaggc 120
agaggagaag gagcaaagaa agaaggaaag ggggagggag ggagaggagg gagggaggga 180
gggagggagg gagaggaggg gagggggaga aagaagagaa ggagagatct tttccccact 240
gactatctca ggaaattacc acaggtggaa gggggtacta attaaggaat agctgtaagt 300
aacattattt ttattcgtag aacctcataa ctcttaagat gtgcttttta cccttttctg 360
ccttttagca caaataaact ccaacatgaa aattatccac tgtgcgtgtg aaaataccta 420
cacagagttc tgaatcattt gccaaattca agccccaatt tttatttcca ttttgactga 480
gagcaagatg ttccttttag gggatggaag cgtctgggtt tcccacactg aatgactcaa 540
ctcgaatgtt gcctcattaa cattctcgat ttttccgtaa tctctgctcc atgcattcaa 600
gataactgtg cctatcacaa atggcttttt agcagctcca ctctttctgg ggatgtggtg 660
gcccttccag tagctgccac cacggattgt cttcaatttc tcacttgttt ttgagttgag 720
tgtcagcctg acccctgggc atggccgcac atgactcagg caaagtgaga gtttcatcac 780
taaacgtggc tctgtttgct atgtctgttt tccctctaag agcaggttat tcaaatacca 840
tctggctgag gtcaagttgc ctcagagccc acagaatctc tacccaggtc cctgttggat 900
ccctaaaaac tcagtcatgc tgtaatctcc ttctgaaact gtgcaatgcc tgcaggctgt 960
cagcccagct ctctccttct gcttcctgtc ctcctaggac cccatgcctc ctcaaacgtc 1020
cacgtgtttc ttgctcctcc accacggttg ccaagccaaa attcgggtgg gcgggaggac 1080
attttcccaa gtgcctgttt cccttctttt ccttttgaca ccccagataa atcatctttc 1140
ccaatccaac acagccccac tgtgtctttg gggacttcat gacatcaccc aggaatgtat 1200
ccttagaaac aaaaatgcaa aacccagaac accaggagac aattaaagaa attttcactg 1260
gtgaggtcac aagtagtaga gacttcttgt taacgggcag aaactttcac ggacccagca 1320
tgctactgtg gcagttctgc aacaagctga aaatgccttt cccgaccacc caagccagtg 1380
ccacacaaag gccaccttag ggtgtgcaca ggatgtcact aggcgttggc ggaactcagg 1440
aaggagtctg aatttcttcc cgtttcttcc ttcctctctc attccctatc ttagcttctg 1500
tctctctttc ctctctctcg ttccccccct tcctccctcc cttcctgttg cagggccaca 1560
gatggaccgg gagacctcaa gcatgtcaaa tcaactaact gctctaccac tcaaccacac 1620
cctcgcctgc attgttacta ctactattat tatcttgata caggtctcca cattgagctc 1680
accctcacag tctccacatt gagctcaccc tcacagtctc cacattgagc tcaccctcac 1740
agtctccaca ttgagctcac cctcacagtc tccacattga gctcaccctc acagtctcca 1800
cattgagctc accctgtggc tctggcaaac cttgaattct ctcattcctc ctgcctcagc 1860
ctctggggtc gtggggatta gccaaaccca cttgaggttt tcttcaatca gcaaattctt 1920
agcgttcaat taacacacac tcataactcc agtactttgg aaaccggaac aggagaattt 1980
ctgtgagctg gaggctagct tggactacag tatgagaccc tgtctctaaa taaatacaca 2040
aagaaatctc accaagggcc tccctctctc agcaagctct aactgtggtg ggagttctgg 2100
gttgttccag ttaacgggct cagaactcta ctgcccagca catcagcccc tagacacagg 2160
tggctctcta catgtgaaca tgcagtcaca gaaatgaaat aaagtgaaaa ttttatttct 2220
tcagttgtat agcctcttcc gtgtgggctg tagttactgt cttgaatagg ataggctcag 2280
aatccttggt gctggaacca agagtttgat tccattagac gacagggaat ataatgccca 2340
atagggcatt cctcctcccg gtcactagcg gtgcactttc tccgaatctt tgtcatgttg 2400
aattagaaaa gttagtattt tcctccatcc cttcccctcc tcccctcctc ccctcctccc 2460
ctcctcccct cctccctccg tctccccgcc cctcccctcc ccctctgatc ctcccccatc 2520
tatcaaatcc aagaattcca gtaaaaagag gaaaacaatc gaagtgattt cgttgattgt 2580
cagttccacc aaagcaagac ttgactttag ttccgcgttt cggttcccgg catgcaccac 2640
agccagcgag caccgtggaa ggatgctagc acggtcctcc ccccgccccc actagctgtc 2700
ttcagctccc cagtagaggg caaccgcact ccagattctc aatggagagt gtttacacaa 2760
tcgttgcggg tttgtgtgag cgcgcccgct tccagagaca cttcttcttt ttcttttttc 2820
catttcatcc cagtggcaac gcagagtgcc agatcattca ggccgtttgc agggcaagcc 2880
gtgggagctt ggcaagcaag gccccatttc ctagggaacc cgtgcctggc gcttcaggaa 2940
agcacgggaa cctggcactg tgactctgcg ggtattattt tgcagaactc tttattaaac 3000
gggagtttca agtccagctg gagacgacca ggcagcgcct ttaaccccag agtcacacac 3060
aggtgccttt tcttggggcc agattggggt tgtgtggcag acctgcgacc agcttgacaa 3120
ctcttctgcc aggccacaaa atggtgttgg ctgtaagagg tgacaccagg gacagggaag 3180
atcgctgcta ttctcctgag ctctccaaag acccacacca gtctgtcccc ctttcctcct 3240
gctcttcccc tgtatcgccc cctcaccatc tcccccaacg agactcttgg catctcctcg 3300
gcacaaggat ttgaaaatag atgcttgggg gtgagaagaa gaagagagaa agagagagaa 3360
ggaaggaagg atatatagat gatacagacg catacaggtg acatgtagct aatcattttt 3420
aattaaaaaa taaattaaaa gcaaatcaag gatatatatg atacccttag agcaagtgtc 3480
tcatacacac acaaacacac acacacaata tatatatata tatatatata tatatatata 3540
tatatatata ttatacttgg aacaagtgtc cagaagggct ggggactcta aagtgcttgt 3600
caaagccagg ctcacatcag taatcttatc acctggtaga ctgagacagg aggattttga 3660
tgagttcagg cccagcctga gctgcagaat gtgattctat cccaaaaaag taaaataaaa 3720
taaaattcaa aatacacgaa aagagtattt gctgaacaaa caagcctaaa gccctggatc 3780
ccttccccca tgtcctaaga aaataagttt cttgaagctg gagggatggc tcagaggtta 3840
agagccccag ctgcacttgc ggaacactaa gacccagttc ccagacccca cactgtgggt 3900
cacaactgtc tcaaacgcca gctccggagg atccatgccc tctcctggcc tccaccggca 3960
ccaagaacac atacagtgcc catacattta tgcaagcaag gtattcacgc acataaaact 4020
aaaagaatat ttaataaaga tataacaaaa tagcatgaag cccagctggt acagaggttc 4080
aaactacatc ccaggttcat ccctctgcct ttgctctcag ttggcttggg taggtctctt 4140
ctctgaactg gcgccctgcg ggttccacat tgagaccctc tcatttttaa acctacttct 4200
tctgggcggg gttaattgct gccagggctc aagccaacgc ttcctcttct ccacagcaat 4260
cttccaagtt tcacgagata accaggaact gctaagttca tgtgaacctt agtgaagaac 4320
ctgagtcttc ccatgtgatt ggtgtgtgca tgtgtgcata cacaaatgta tgtgtgtgct 4380
ctatgtgtgc ctatgtatgt gtgcatgcat gtgtgcatat acaaatgcat atatgtctat 4440
gtagtgtgcg tacacaaatg tatgtgtgtg ctcaatgtgt gcctatgtgt gtgtatgcat 4500
gtgtgcgtac acaatgcatg tgtgtggtgt ctgtgtgcct gtgtgtgtat gcatgtatgc 4560
atacacaaat gtatatgtgt ggtgtgtgaa tgtgtgccta tgtatgtgtg tgctgtgtgt 4620
gggtgtggta tgtgtgtgat gtgtggaggg gtgtgtatgt gtggtatgta taggtgatac 4680
gtttggggtg taatatgcgt atgtggtttg tgaaatgtag ttcgtgtgtg tgcatgtgtg 4740
cgtgcgtgcg tgcgtgcgtg cgtgtgtgtg tgtgtgtgtg tgtgtgtgtt ggatatagta 4800
tgtgtgaggt gtgtgtactc accatggcct ccctcacttg ggggagtgaa gtcagcagcc 4860
tggaccactc agggacatga gatactcaga cacatcttga tttccacccc tcttttcctg 4920
atcctccttc acgtgtcact ttcccaaaca ctggacaaca gtttgggggc atctgattcc 4980
actaatgaca gggacatcac atgtctccag agggaacacc ttctgtgtca catgtcatct 5040
gagaatgtag cagagtcaca gagaaatgtc acagaaacca aaatgcagag taccaaggta 5100
tagctaggca cagagcagag gggaagccgc tgaatttatt aaaaatgtca gaatcgtaaa 5160
agacagggga cagcggtggg gacattcagg gtccagtagc acacaggcag tccaaacctg 5220
atcactggaa ggtagtaggt aaggaaaggc tgcacacaga ttattcacac agtttataca 5280
tgtacacaga ttattcacat ggtttgtgta tgtgcacaga ttattcacac agtttataca 5340
tgtgtggctt cgtggtaact ttgagcttac tttcaattta aaaggatctc tctcacaagc 5400
tggggccggg aatggctgca gtcaacactc catcacttag tcacactgtg caaacagcac 5460
ctcctgactc atggtgactt gtagtaaaat gaagaggcca catttgcatc caagacagct 5520
catcagtacc tagtgaagaa tctgtccctg agtatttgca tgaatggacc cgggtccagg 5580
gcctggctgg gagtctccag gtgttgcagc cagaatgtca ttgtgttttt tcaggatccc 5640
agaagtttct aaaatacagg ccaagtactc atttgtgtta caaagtatct gactaataga 5700
agtgattagg taacacaaag ccttttaaaa accgagatca cccttgtcat gtccctggcc 5760
tcttagaaca agatccaagc ttttgctggt tgacaagtgg ggccatccag tgcgtctccg 5820
ttcctgctac ttcatctgga agacctctcc cactaacttg cccctgaccc ctcacacctg 5880
ctgtttcctt tccacccgga agtgcttgtc taggctttca tggccatctg actgagcatc 5940
taggcctcag tccagtggtc cctcagctct ctctagtcac tgtactaatg gaaacggcca 6000
ctaactacat tttcaatatg gaagcctcct cctcaggaac ctccaagggc agaagcctcc 6060
agagaaccac tcctgacccc ctggagttct gagtgcttct ggccctctct gtgtctgcag 6120
gactattcac cacttgtgtt gaatggttca gtcctcacct cctctggcat gtgctcagtt 6180
ctcatctcat tggggagtcc ttcccaggtc actcttctct cctgtctttg aagtgttttt 6240
ttccttcatg gtatttctgt ctgggcacac acacagacac acatacacac acatacacac 6300
ccatgcagta tggcagatac atcacctatg tttcagattt ttattctacc atcacccaat 6360
acctgaatcc ccgaaaaagc cttagaaagc caggaatttg tgtatttttg tcagcactcc 6420
accccagcac ctgaagccaa gcctgactta atatttttgg ttttgtttct aga 6473

<211> 7045

<212> DNA

<213> Cricetulus griseus

<400> 4 (SEQ ID NO: 14)

caattgatta tagatggatg atagatagat agatagatag atagatagat agatagatga 60
tggatagaca gatgatggat agttagagga tagataatga ctgaataata agtacataaa 120
tagatgatag agcggggcgt tggtggtgca cgtctttaac cccagcacca gagaggcaga 180
ggcagttgga tctctgtgag tttgaggaca gcctggttac agaatgggtt ccaggacagc 240
caaggctgtc actcagagaa atactgtctc aaataaaaaa agtaagtaaa caaataaata 300
aatgataact agttagaaga tagatgattg aatgataggt agataaatag aagatagata 360
gatagatgat tgatagatga tagacagata gacagacaga cagacagaca gacagcagaa 420
agataatgca cggtgaaaca tggtctgatt tagttagcaa gatcagagaa gccttctttg 480
aaagtgacat ttgagagcat ttcaaacgct gttcatgtca ggcatgccaa tggggagaga 540
agggcttgca gaaagcaggc ccggcaagcc atggggagca agctaggagg cagcattcct 600
tgcatttgcc tctgcctcag ctgcttcctg gagttccccg gtttttatca caacagtaga 660
aataaaacca ggacaatgtt gtttccatgc atacatctgc aagaacttac tccggttcaa 720
tagacagacc aaggcacctg tgtttgctca agaagcacgg agggaggtgt gtgcacctgc 780
tgggtgctgg tgctctggct gtgccagaca gagagcaaga caggaaagtt cctggtggcc 840
tagagcacac agcccagccc aggaagtcat gtctctctct gtctctgtct ctgccccacc 900
cccaccccat ttaggccaga gaacagctgt ggcaagcttt gggtttgggt gagtcattcc 960
tcaagagcca agagccgccc accttgtatg gggtagtttg ttgttgttgt tgttgttatt 1020
atttgtttgt ttgtttgttt ggtaaaggtt tttcaatagg agttggaatt tggcaattca 1080
gctaggctgg ctgagcagcc agctagcccc gggcactcat ccgtctctac ctccccagtt 1140
ctgggatttc gggtacatgc tgccacatcc gacttttttc ccctgctcca gttcttaaga 1200
ccaagtcttc atgtcaaaca cttcaccacc ttagccatct ttctgggtca gaagttagat 1260
cttcaggaag acaaggagtg tatcaggaca tgagcgtgcc ccaactctgc tcagaccttc 1320
tgatagagaa aatgggggga ggggtgtcag aggctgccgg agaaagacaa gtccaggtta 1380
aggaggacga ccctgggctc tgaatccaag ggtgattccc tcaccttgta cacttggcat 1440
tttgggaagg aagcatcaga taaaagcagt gcagacatag tcaggaatat ttacacgtgt 1500
gagtcaacct gggagtgagt ctgtgtacaa ctgaacatga agcaagtttt gaagcttcat 1560
ttccagacta ttcccagggt gcaataactt cctgttttcg ttgcagcctt cccagtctct 1620
gccactgcca tctctacttc agtctggaat ggtgggcaca cagaaaaagt ctatggcaat 1680
cctgcgagaa gacaagtggg cgcctgactt cgggctcctg ttacaagaga ggaatccagg 1740
agtttatttt gcagctgatt cagtgttgac caagagtcca gctctggggg agtgggaagc 1800
aaccaaagca gagacaggtc ccagcacaat ttttggtttt caagacagca cttctctgtg 1860
gctttgaagg ctatcctaga actgttcttt gtatatcctt ccttgcaact agctcttata 1920
gaccaggctg gtcttgaact cacagagatc catctgcctc tgcctcccaa gtgctgggat 1980
taaaggcgtg cacctcggct gccaccaccc agctacatac ataatttaca ataataaaaa 2040
taaaatactt taaagtgtta tagcagtttg aatgtaattg gccctgtcat ctcataggga 2100
gtggcactat taggaggtat ggctttgttg aaggaaatat gtcactgtga gggtgggctg 2160
tgaggtttcc tatgctcagg gtaccagcca gtgtctcagc tgaggtcctg ttgcctgcaa 2220
gatgtaggac tctcatccct ttctccagca ccatgtctgt ctgcatgcca tcatgttccc 2280
agccatgatg acaatgtact aaaacctctg aaactgccac ccaactaaat gttttccttt 2340
ataagagttg ccatgctcat ggtgtctctt cacagcaata gaaaccctaa ctaagataag 2400
tgtattctcc cctactcccc atgatttaaa atttaggaag gcaggtaggc aggcaggcag 2460
gctggtatag tggttcattc tagcacctga gacctggaat gggaggattg tgagttagtt 2520
ctaggccatt ctggtgccta gaaaccagag ccgggggttg gcccaatgca gagcacttgc 2580
tctacgtatg gcccagcaca ataagtcaat ttcctcacct taaaggcttg acaatttaaa 2640
aacactggtt tttagttagt ccgtgtctgc tccacagatg gagacagcta atcacagatg 2700
catcaggggc cttcctgagt gctaaacatc aaacagcctt ctcccctcct gagcctttgt 2760
gtgcagaatg tgtccatcgc aagaagcaaa cagtcttgct tgcccaccaa cttccttcct 2820
gcatcagaag agctgggtgc aaactgcaag agtagcctca ccttagagat gggtcccatt 2880
gctctacatg ggagcattac cttccaagaa ggcaaaaatg tctcctggtt gagctttttt 2940
tgtcacctgt taaaggcaaa tcaacagaga ggctttgtct cacccactaa catcttggaa 3000
acaaatacca acgaacgctg gggaggatgt ggggaaagca gagccctcat gctctccgag 3060
ggaaaatcac acccactgtg gaacagtgtg gaaacctcaa agactgggat tacaagcagc 3120
acacaagcca gccacgctac tcctggtcac acaccacaaa gacgcttgca cattcacgct 3180
tacgctgcga acactagcaa cgttcccact gcctcctttg agccccgccc cccgcccctg 3240
ccccccgccc cgcccctgtg gtctatgttc ctcttcccta aagtcagctt ccacttctct 3300
gtctccatct tcgccccacc ctccctcctc gctacataat tgtctctatt ccatttctct 3360
gctttgaaac agctttttgc aaagcatcaa atctattgtc ctatgcccca aatcaacctc 3420
cagtttcaca agtgatacag gaaatcgttt tcctaattaa aaatcccccc tttgaccatt 3480
tattcccact cttggaacat cttccccttg aggaaagtta cagaatgagg tggctctcct 3540
cttcctattc gaggtgtttc cttcagactt tgtccgtgtc taatcttttt aactgttggc 3600
caggcctcca ccacggcaca gatgaactgt ggggttcatt tacctgaaac tctatggaag 3660
gatgtttatt tctccttcac tttagcaaat gataaagggc accattcact ctgtctattc 3720
tgcaggggcc attcctttct ctaggccaga tactgagaat tgctcccaga atcaatgtgg 3780
tatacatatt tccccttcaa cattgatagg cattgatcac acacacacac acacacacac 3840
acacacacac acacagtagc acaaatgtat tcccctagcc cgcttccatc ttgccacagg 3900
actccagagt ggccctggat agcaagcttc ctgttttgtt tctctgttcc tgctgctttt 3960
ccaccctcca gtctatcttt tctaagtcct tctgccattg tcctcttccc aactgtcctg 4020
agatgcagtc attgtctggg attcagacct tctctctctg cccaagtgag tatattgacc 4080
cccacggttt gtacaaccat aacttcaggg agcccgacaa aaactgtttt atgagccaag 4140
tagtcccagg acttgagagg tagaggcggg aagatcagca gtttgaggcc agcctggaga 4200
gcataagagc cggtctcaaa acaacaatgg aaactagata ctaagtaaaa atcctggggt 4260
gtttcatcat gaatgtctgt tcttctagta ccacgctgaa ctccgtacac agctccagct 4320
gttacggctt tcttagaatc catactcttt tttttttttt tttttttttt ttttttttgg 4380
tttttcgaga cagggtttct ctgtggcttt ggaggctgtc ctggaactag ctcttataga 4440
ccaggctggt ctcgaactca cagagatcca cctgcctctg cctccagagt gctgggatta 4500
aaggcgtgcg ccaccaacac ccggcagaat ccatactctt tttaaaaaaa gatttatcaa 4560
tttactatgt atacagcttt ctgcctgcat gtatccatgc atgtcagaag atggcaccag 4620
gtcgcattac agatggttgt gagccaccat gtggttgctg ggaattgaac tcagaatgtc 4680
tagaagagca accagttctc ttaacctctg agccatctct ccggccccca gaaatccata 4740
ttcttgagga ttttttacac cccccccacc aaaagacgta tatctaaatt ttaatgtgag 4800
aattcacatt ttcttaagag ttgaacatag atttagagga aaatcagatc ccacatgatt 4860
aacaaagcat gcttgtgggc aggtctgcta ccaagaggtg ggccgtagct tctagctcag 4920
acaaactcac tcccttcctc gtggcctctt cgccctcaag tcagaaactc accctgtgat 4980
tctgccccag aagttgctct agagcacagt gcatccttcc gtcttcactc tgtggcttga 5040
attgtgtcca tcgcttatga ttacaacccc tcacagagca tcctaactgg tttctttgca 5100
tgcctatggg cactcctcca ttctagaaca cccttgccat caatactatg aaaggagggg 5160
tggaggagga agagcaggaa gaggaggggg aagcgaggga agaggaagac acggatggca 5220
atgaggaggg gggagcaccc aagtcctccc tggatgagag tctcactggg agacttaata 5280
ttaattataa atgcttggtc agcagctggg caggataagg ttaggcagga gaaccagact 5340
aaggactctg ggaagcagaa gggcagagtc agacaaggag aggaaacagg aagtacaagg 5400
taaagtcacg tggcagaatg tagataatag aaatgggttc atttaagttg gaagagttag 5460
ctagtaacaa gcctgagcta tcagccgagc atttataatt aatattgagc ctccatattg 5520
gttatctggg aattggcggg cagaaaaaaa aaagtctgcc tacaagtcaa tgtcatgtag 5580
ctcccaaagc caaggtacct ttgttcagtg cttgactgag ccagcattat aaattttctc 5640
cagatgtacc gaatcacatt tcatagcaac atgcagacat caagttttcc ctgaagctct 5700
aaccagctgg ttgcatgctg tccggagtct cagctataac ccagaagtga cctgggtcgg 5760
ggaagaggtg gtactttgcc ttctttgcac tctctgtgtt gcctcaccca ttcagcttca 5820
agcaatgtga ctgcctgacc ctgagggcgt ttacaacgcc tgacccacag accacaagtc 5880
aaccagctgg tgtgctcacg atacctagtc tgaaccatag ccctgctccc accctgcctc 5940
catctccacc ctttcttcac tgctcatcac agctggctag caaagactgc ctcagacctg 6000
agcacaggct ccactccaca gccgtgactg ttcgagccac ttaaatcaaa gagcgcttgt 6060
cttccgctca gtaaatctct cctcagctca ctgatgacgt tgactttctc tagacagcac 6120
atttgggttt aagacactgc tacttgagct cttcattcag ttcctcagaa tacctcattt 6180
gggtcagatt cccaaagagg aagatagggt tcctggcaga cagacatgtc tcattccttt 6240
gaaatccttc agagaaatgc agtgactatg gcaccttctt aaaaagcaca cacacaaata 6300
acacacacac acacacacac acacacacac acacacacac atatccccct cactgtcatc 6360
cttgatatgt atatgatata tataaaatca ttgttttata ctgtgataat tgattatgaa 6420
taaaatttac taaaatgaac aattaaaatt atgggggggg ctggagagat ggctcatcag 6480
ttaagagaac agttgctgct cttgcagaac acgagagttc agttcccagc acccacatca 6540
ggcagctcat aaccatgtgt ggtgtcagtt ccaggagatc tggtgccctc ttctggcctc 6600
ctccagcacc tgctacatgt ggttcacaca cacacacaca cacacacaca cacacacaca 6660
cacacacaca caaataaata taaagattat ttttttcaaa actgagttaa aaataggttc 6720
tatctgattc atactaaggc ttttcacagt ggttaagtct attagatatg tctagccata 6780
tcctttctcc cttctttctt gaggagaggc ttttaaagct acaagttaca gccttctttg 6840
caaataagag taccatttaa caggcctctg accaatgaga tgccagaatc ggttgcccag 6900
gagcttccca aacagtccat tatagggaaa ggtggtacaa accagtagat taggcatgtt 6960
ccacttccta agtgccgtgc caaataagga aatggcctca aatgtttgcc ttttatcttc 7020
acccacctct gaattgcacg ctagt 7045

<211> 13515

<212> DNA

<213> Cricetulus griseus

<400> 5 (SEQ ID NO: 15)

tctagaaaca aaaccaaaaa tattaagtca ggcttggctt caggtgctgg ggtggagtgc 60
tgacaaaaat acacaaattc ctggctttct aaggcttttt cggggattca ggtattgggt 120
gatggtagaa taaaaatctg aaacataggt gatgtatctg ccatactgca tgggtgtgta 180
tgtgtgtgta tgtgtgtctg tgtgtgtgcc cagacagaaa taccatgaag gaaaaaaaca 240
cttcaaagac aggagagaag agtgacctgg gaaggactcc ccaatgagat gagaactgag 300
cacatgccag aggaggtgag gactgaacca ttcaacacaa gtggtgaata gtcctgcaga 360
cacagagagg gccagaagca ctcagaactc cagggggtca ggagtggttc tctggaggct 420
tctgcccttg gaggttcctg aggaggaggc ttccatattg aaaatgtagt tagtggccgt 480
ttccattagt acagtgacta gagagagctg agggaccact ggactgaggc ctagatgctc 540
agtcagatgg ccatgaaagc ctagacaagc acttccgggt ggaaaggaaa cagcaggtgt 600
gaggggtcag gggcaagtta gtgggagagg tcttccagat gaagtagcag gaacggagac 660
gcactggatg gccccacttg tcaaccagca aaagcttgga tcttgttcta agaggccagg 720
gacatgacaa gggtgatctc ggtttttaaa aggctttgtg ttacctaatc acttctatta 780
gtcagatact ttgtaacaca aatgagtact tggcctgtat tttagaaact tctgggatcc 840
tgaaaaaaca caatgacatt ctggctgcaa cacctggaga ctcccagcca ggccctggac 900
ccgggtccat tcatgcaaat actcagggac agattcttca ctaggtactg atgagctgtc 960
ttggatgcaa atgtggcctc ttcattttac tacaagtcac catgagtcag gaggtgctgt 1020
ttgcacagtg tgactaagtg atggagtgtt gactgcagcc attcccggcc ccagcttgtg 1080
agagagatcc ttttaaattg aaagtaagct caaagttacc acgaagccac acatgtataa 1140
actgtgtgaa taatctgtgc acatacacaa accatgtgaa taatctgtgt acatgtataa 1200
actgtgtgaa taatctgtgt gcagcctttc cttacctact accttccagt gatcaggttt 1260
ggactgcctg tgtgctactg gaccctgaat gtccccaccg ctgtcccctg tcttttacga 1320
ttctgacatt tttaataaat tcagcggctt cccctctgct ctgtgcctag ctataccttg 1380
gtactctgca ttttggtttc tgtgacattt ctctgtgact ctgctacatt ctcagatgac 1440
atgtgacaca gaaggtgttc cctctggaga catgtgatgt ccctgtcatt agtggaatca 1500
gatgccccca aactgttgtc cagtgtttgg gaaagtgaca cgtgaaggag gatcaggaaa 1560
agaggggtgg aaatcaagat gtgtctgagt atctcatgtc cctgagtggt ccaggctgct 1620
gacttcactc ccccaagtga gggaggccat ggtgagtaca cacacctcac acatactata 1680
tccaacacac acacacacac acacacacac acgcacgcac gcacgcacgc acgcacacat 1740
gcacacacac gaactacatt tcacaaacca catacgcata ttacacccca aacgtatcac 1800
ctatacatac cacacataca cacccctcca cacatcacac acataccaca cccacacaca 1860
gcacacacat acataggcac acattcacac accacacata tacatttgtg tatgcataca 1920
tgcatacaca cacaggcaca cagacaccac acacatgcat tgtgtacgca cacatgcata 1980
cacacacata ggcacacatt gagcacacac atacatttgt gtacgcacac tacatagaca 2040
tatatgcatt tgtatatgca cacatgcatg cacacataca taggcacaca tagagcacac 2100
acatacattt gtgtatgcac acatgcacac accaatcaca tgggaagact caggttcttc 2160
actaaggttc acatgaactt agcagttcct ggttatctcg tgaaacttgg aagattgctg 2220
tggagaagag gaagcgttgg cttgagccct ggcagcaatt aaccccgccc agaagaagta 2280
ggtttaaaaa tgagagggtc tcaatgtgga acccgcaggg cgccagttca gagaagagac 2340
ctacccaagc caactgagag caaaggcaga gggatgaacc tgggatgtag tttgaacctc 2400
tgtaccagct gggcttcatg ctattttgtt atatctttat taaatattct tttagtttta 2460
tgtgcgtgaa taccttgctt gcataaatgt atgggcactg tatgtgttct tggtgccggt 2520
ggaggccagg agagggcatg gatcctccgg agctggcgtt tgagacagtt gtgacccaca 2580
gtgtggggtc tgggaactgg gtcttagtgt tccgcaagtg cagctggggc tcttaacctc 2640
tgagccatcc ctccagcttc aagaaactta ttttcttagg acatggggga agggatccag 2700
ggctttaggc ttgtttgttc agcaaatact cttttcgtgt attttgaatt ttattttatt 2760
ttactttttt gggatagaat cacattctgc agctcaggct gggcctgaac tcatcaaaat 2820
cctcctgtct cagtctacca ggtgataaga ttactgatgt gagcctggct ttgacaagca 2880
ctttagagtc cccagccctt ctggacactt gttccaagta taatatatat atatatatat 2940
atatatatat atatatatat atatattgtg tgtgtgtgtt tgtgtgtgta tgagacactt 3000
gctctaaggg tatcatatat atccttgatt tgcttttaat ttatttttta attaaaaatg 3060
attagctaca tgtcacctgt atgcgtctgt atcatctata tatccttcct tccttctctc 3120
tctttctctc ttcttcttct cacccccaag catctatttt caaatccttg tgccgaggag 3180
atgccaagag tctcgttggg ggagatggtg agggggcgat acaggggaag agcaggagga 3240
aagggggaca gactggtgtg ggtctttgga gagctcagga gaatagcagc gatcttccct 3300
gtccctggtg tcacctctta cagccaacac cattttgtgg cctggcagaa gagttgtcaa 3360
gctggtcgca ggtctgccac acaaccccaa tctggcccca agaaaaggca cctgtgtgtg 3420
actctggggt taaaggcgct gcctggtcgt ctccagctgg acttgaaact cccgtttaat 3480
aaagagttct gcaaaataat acccgcagag tcacagtgcc aggttcccgt gctttcctga 3540
agcgccaggc acgggttccc taggaaatgg ggccttgctt gccaagctcc cacggcttgc 3600
cctgcaaacg gcctgaatga tctggcactc tgcgttgcca ctgggatgaa atggaaaaaa 3660
gaaaaagaag aagtgtctct ggaagcgggc gcgctcacac aaacccgcaa cgattgtgta 3720
aacactctcc attgagaatc tggagtgcgg ttgccctcta ctggggagct gaagacagct 3780
agtgggggcg gggggaggac cgtgctagca tccttccacg gtgctcgctg gctgtggtgc 3840
atgccgggaa ccgaaacgcg gaactaaagt caagtcttgc tttggtggaa ctgacaatca 3900
acgaaatcac ttcgattgtt ttcctctttt tactggaatt cttggatttg atagatgggg 3960
gaggatcaga gggggagggg aggggcgggg agacggaggg aggaggggag gaggggagga 4020
ggggaggagg ggaggagggg aagggatgga ggaaaatact aacttttcta attcaacatg 4080
acaaagattc ggagaaagtg caccgctagt gaccgggagg aggaatgccc tattgggcat 4140
tatattccct gtcgtctaat ggaatcaaac tcttggttcc agcaccaagg attctgagcc 4200
tatcctattc aagacagtaa ctacagccca cacggaagag gctatacaac tgaagaaata 4260
aaattttcac tttatttcat ttctgtgact gcatgttcac atgtagagag ccacctgtgt 4320
ctaggggctg atgtgctggg cagtagagtt ctgagcccgt taactggaac aacccagaac 4380
tcccaccaca gttagagctt gctgagagag ggaggccctt ggtgagattt ctttgtgtat 4440
ttatttagag acagggtctc atactgtagt ccaagctagc ctccagctca cagaaattct 4500
cctgttccgg tttccaaagt actggagtta tgagtgtgtg ttaattgaac gctaagaatt 4560
tgctgattga agaaaacctc aagtgggttt ggctaatccc cacgacccca gaggctgagg 4620
caggaggaat gagagaattc aaggtttgcc agagccacag ggtgagctca atgtggagac 4680
tgtgagggtg agctcaatgt ggagactgtg agggtgagct caatgtggag actgtgaggg 4740
tgagctcaat gtggagactg tgagggtgag ctcaatgtgg agactgtgag ggtgagctca 4800
atgtggagac ctgtatcaag ataataatag tagtagtaac aatgcaggcg agggtgtggt 4860
tgagtggtag agcagttagt tgatttgaca tgcttgaggt ctcccggtcc atctgtggcc 4920
ctgcaacagg aagggaggga ggaagggggg gaacgagaga gaggaaagag agacagaagc 4980
taagataggg aatgagagag gaaggaagaa acgggaagaa attcagactc cttcctgagt 5040
tccgccaacg cctagtgaca tcctgtgcac accctaaggt ggcctttgtg tggcactggc 5100
ttgggtggtc gggaaaggca ttttcagctt gttgcagaac tgccacagta gcatgctggg 5160
tccgtgaaag tttctgcccg ttaacaagaa gtctctacta cttgtgacct caccagtgaa 5220
aatttcttta attgtctcct ggtgttctgg gttttgcatt tttgtttcta aggatacatt 5280
cctgggtgat gtcatgaagt ccccaaagac acagtggggc tgtgttggat tgggaaagat 5340
gatttatctg gggtgtcaaa aggaaaagaa gggaaacagg cacttgggaa aatgtcctcc 5400
cgcccacccg aattttggct tggcaaccgt ggtggaggag caagaaacac gtggacgttt 5460
gaggaggcat ggggtcctag gaggacagga agcagaagga gagagctggg ctgacagcct 5520
gcaggcattg cacagtttca gaaggagatt acagcatgac tgagttttta gggatccaac 5580
agggacctgg gtagagattc tgtgggctct gaggcaactt gacctcagcc agatggtatt 5640
tgaataacct gctcttagag ggaaaacaga catagcaaac agagccacgt ttagtgatga 5700
aactctcact ttgcctgagt catgtgcggc catgcccagg ggtcaggctg acactcaact 5760
caaaaacaag tgagaaattg aagacaatcc gtggtggcag ctactggaag ggccaccaca 5820
tccccagaaa gagtggagct gctaaaaagc catttgtgat aggcacagtt atcttgaatg 5880
catggagcag agattacgga aaaatcgaga atgttaatga ggcaacattc gagttgagtc 5940
attcagtgtg ggaaacccag acgcttccat cccctaaaag gaacatcttg ctctcagtca 6000
aaatggaaat aaaaattggg gcttgaattt ggcaaatgat tcagaactct gtgtaggtat 6060
tttcacacgc acagtggata attttcatgt tggagtttat ttgtgctaaa aggcagaaaa 6120
gggtaaaaag cacatcttaa gagttatgag gttctacgaa taaaaataat gttacttaca 6180
gctattcctt aattagtacc cccttccacc tgtggtaatt tcctgagata gtcagtgggg 6240
aaaagatctc tccttctctt ctttctcccc ctcccctcct ctccctccct ccctccctcc 6300
ctccctcctc tccctccctc cccctttcct tctttctttg ctccttctcc tctgcctcct 6360
tctccctttc ttcttcattt attctaagta gcttttaaca gcacaccaat tacctgtgta 6420
taacgggaaa acacaggctc aagcagctta gagaagattg atctgtgttc actagcgtgc 6480
aattcagagg tgggtgaaga taaaaggcaa acatttgagg ccatttcctt atttggcacg 6540
gcacttagga agtggaacat gcctaatcta ctggtttgta ccacctttcc ctataatgga 6600
ctgtttggga agctcctggg caaccgattc tggcatctca ttggtcagag gcctgttaaa 6660
tggtactctt atttgcaaag aaggctgtaa cttgtagctt taaaagcctc tcctcaagaa 6720
agaagggaga aaggatatgg ctagacatat ctaatagact taaccactgt gaaaagcctt 6780
agtatgaatc agatagaacc tatttttaac tcagttttga aaaaaataat ctttatattt 6840
atttgtgtgt gtgtgtgtgt gtgtgtgtgt gtgtgtgtgt gtgtgtgtgt gaaccacatg 6900
tagcaggtgc tggaggaggc cagaagaggg caccagatct cctggaactg acaccacaca 6960
tggttatgag ctgcctgatg tgggtgctgg gaactgaact ctcgtgttct gcaagagcag 7020
caactgttct cttaactgat gagccatctc tccagccccc cccataattt taattgttca 7080
ttttagtaaa ttttattcat aatcaattat cacagtataa aacaatgatt ttatatatat 7140
catatacata tcaaggatga cagtgagggg gatatgtgtg tgtgtgtgtg tgtgtgtgtg 7200
tgtgtgtgtg tgtgttattt gtgtgtgtgc tttttaagaa ggtgccatag tcactgcatt 7260
tctctgaagg atttcaaagg aatgagacat gtctgtctgc caggaaccct atcttcctct 7320
ttgggaatct gacccaaatg aggtattctg aggaactgaa tgaagagctc aagtagcagt 7380
gtcttaaacc caaatgtgct gtctagagaa agtcaacgtc atcagtgagc tgaggagaga 7440
tttactgagc ggaagacaag cgctctttga tttaagtggc tcgaacagtc acggctgtgg 7500
agtggagcct gtgctcaggt ctgaggcagt ctttgctagc cagctgtgat gagcagtgaa 7560
gaaagggtgg agatggaggc agggtgggag cagggctatg gttcagacta ggtatcgtga 7620
gcacaccagc tggttgactt gtggtctgtg ggtcaggcgt tgtaaacgcc ctcagggtca 7680
ggcagtcaca ttgcttgaag ctgaatgggt gaggcaacac agagagtgca aagaaggcaa 7740
agtaccacct cttccccgac ccaggtcact tctgggttat agctgagact ccggacagca 7800
tgcaaccagc tggttagagc ttcagggaaa acttgatgtc tgcatgttgc tatgaaatgt 7860
gattcggtac atctggagaa aatttataat gctggctcag tcaagcactg aacaaaggta 7920
ccttggcttt gggagctaca tgacattgac ttgtaggcag actttttttt ttctgcccgc 7980
caattcccag ataaccaata tggaggctca atattaatta taaatgctcg gctgatagct 8040
caggcttgtt actagctaac tcttccaact taaatgaacc catttctatt atctacattc 8100
tgccacgtga ctttaccttg tacttcctgt ttcctctcct tgtctgactc tgcccttctg 8160
cttcccagag tccttagtct ggttctcctg cctaacctta tcctgcccag ctgctgacca 8220
agcatttata attaatatta agtctcccag tgagactctc atccagggag gacttgggtg 8280
ctcccccctc ctcattgcca tccgtgtctt cctcttccct cgcttccccc tcctcttcct 8340
gctcttcctc ctccacccct cctttcatag tattgatggc aagggtgttc tagaatggag 8400
gagtgcccat aggcatgcaa agaaaccagt taggatgctc tgtgaggggt tgtaatcata 8460
agcgatggac acaattcaag ccacagagtg aagacggaag gatgcactgt gctctagagc 8520
aacttctggg gcagaatcac agggtgagtt tctgacttga gggcgaagag gccacgagga 8580
agggagtgag tttgtctgag ctagaagcta cggcccacct cttggtagca gacctgccca 8640
caagcatgct ttgttaatca tgtgggatct gattttcctc taaatctatg ttcaactctt 8700
aagaaaatgt gaattctcac attaaaattt agatatacgt cttttggtgg ggggggtgta 8760
aaaaatcctc aagaatatgg atttctgggg gccggagaga tggctcagag gttaagagaa 8820
ctggttgctc ttctagacat tctgagttca attcccagca accacatggt ggctcacaac 8880
catctgtaat gcgacctggt gccatcttct gacatgcatg gatacatgca ggcagaaagc 8940
tgtatacata gtaaattgat aaatcttttt ttaaaaagag tatggattct gccgggtgtt 9000
ggtggcgcac gcctttaatc ccagcactct ggaggcagag gcaggtggat ctctgtgagt 9060
tcgagaccag cctggtctat aagagctagt tccaggacag cctccaaagc cacagagaaa 9120
ccctgtctcg aaaaaccaaa aaaaaaaaaa aaaaaaaaaa aaaaaaaaga gtatggattc 9180
taagaaagcc gtaacagctg gagctgtgta cggagttcag cgtggtacta gaagaacaga 9240
cattcatgat gaaacacccc aggattttta cttagtatct agtttccatt gttgttttga 9300
gaccggctct tatgctctcc aggctggcct caaactgctg atcttcccgc ctctacctct 9360
caagtcctgg gactacttgg ctcataaaac agtttttgtc gggctccctg aagttatggt 9420
tgtacaaacc gtgggggtca atatactcac ttgggcagag agagaaggtc tgaatcccag 9480
acaatgactg catctcagga cagttgggaa gaggacaatg gcagaaggac ttagaaaaga 9540
tagactggag ggtggaaaag cagcaggaac agagaaacaa aacaggaagc ttgctatcca 9600
gggccactct ggagtcctgt ggcaagatgg aagcgggcta ggggaataca tttgtgctac 9660
tgtgtgtgtg tgtgtgtgtg tgtgtgtgtg tgtgtgtgat caatgcctat caatgttgaa 9720
ggggaaatat gtataccaca ttgattctgg gagcaattct cagtatctgg cctagagaaa 9780
ggaatggccc ctgcagaata gacagagtga atggtgccct ttatcatttg ctaaagtgaa 9840
ggagaaataa acatccttcc atagagtttc aggtaaatga accccacagt tcatctgtgc 9900
cgtggtggag gcctggccaa cagttaaaaa gattagacac ggacaaagtc tgaaggaaac 9960
acctcgaata ggaagaggag agccacctca ttctgtaact ttcctcaagg ggaagatgtt 10020
ccaagagtgg gaataaatgg tcaaaggggg gatttttaat taggaaaacg atttcctgta 10080
tcacttgtga aactggaggt tgatttgggg cataggacaa tagatttgat gctttgcaaa 10140
aagctgtttc aaagcagaga aatggaatag agacaattat gtagcgagga gggagggtgg 10200
ggcgaagatg gagacagaga agtggaagct gactttaggg aagaggaaca tagaccacag 10260
gggcggggcg gggggcaggg gcggggggcg gggctcaaag gaggcagtgg gaacgttgct 10320
agtgttcgca gcgtaagcgt gaatgtgcaa gcgtctttgt ggtgtgtgac caggagtagc 10380
gtggctggct tgtgtgctgc ttgtaatccc agtctttgag gtttccacac tgttccacag 10440
tgggtgtgat tttccctcgg agagcatgag ggctctgctt tccccacatc ctccccagcg 10500
ttcgttggta tttgtttcca agatgttagt gggtgagaca aagcctctct gttgatttgc 10560
ctttaacagg tgacaaaaaa agctcaacca ggagacattt ttgccttctt ggaaggtaat 10620
gctcccatgt agagcaatgg gacccatctc taaggtgagg ctactcttgc agtttgcacc 10680
cagctcttct gatgcaggaa ggaagttggt gggcaagcaa gactgtttgc ttcttgcgat 10740
ggacacattc tgcacacaaa ggctcaggag gggagaaggc tgtttgatgt ttagcactca 10800
ggaaggcccc tgatgcatct gtgattagct gtctccatct gtggagcaga cacggactaa 10860
ctaaaaacca gtgtttttaa attgtcaagc ctttaaggtg aggaaattga cttattgtgc 10920
tgggccatac gtagagcaag tgctctgcat tgggccaacc cccggctctg gtttctaggc 10980
accagaatgg cctagaacta actcacaatc ctcccattcc aggtctcagg tgctagaatg 11040
aaccactata ccagcctgcc tgcctgccta cctgccttcc taaattttaa atcatgggga 11100
gtaggggaga atacacttat cttagttagg gtttctattg ctgtgaagag acaccatgag 11160
catggcaact cttataaagg aaaacattta gttgggtggc agtttcagag gttttagtac 11220
attgtcatca tggctgggaa catgatggca tgcagacaga catggtgctg gagaaaggga 11280
tgagagtcct acatcttgca ggcaacagga cctcagctga gacactggct ggtaccctga 11340
gcataggaaa cctcacagcc caccctcaca gtgacatatt tccttcaaca aagccatacc 11400
tcctaatagt gccactccct atgagatgac agggccaatt acattcaaac tgctataaca 11460
ctttaaagta ttttattttt attattgtaa attatgtatg tagctgggtg gtggcagccg 11520
aggtgcacgc ctttaatccc agcacttggg aggcagaggc agatggatct ctgtgagttc 11580
aagaccagcc tggtctataa gagctagttg caaggaagga tatacaaaga acagttctag 11640
gatagccttc aaagccacag agaagtgctg tcttgaaaac caaaaattgt gctgggacct 11700
gtctctgctt tggttgcttc ccactccccc agagctggac tcttggtcaa cactgaatca 11760
gctgcaaaat aaactcctgg attcctctct tgtaacagga gcccgaagtc aggcgcccac 11820
ttgtcttctc gcaggattgc catagacttt ttctgtgtgc ccaccattcc agactgaagt 11880
agagatggca gtggcagaga ctgggaaggc tgcaacgaaa acaggaagtt attgcaccct 11940
gggaatagtc tggaaatgaa gcttcaaaac ttgcttcatg ttcagttgta cacagactca 12000
ctcccaggtt gactcacacg tgtaaatatt cctgactatg tctgcactgc ttttatctga 12060
tgcttccttc ccaaaatgcc aagtgtacaa ggtgagggaa tcacccttgg attcagagcc 12120
cagggtcgtc ctccttaacc tggacttgtc tttctccggc agcctctgac acccctcccc 12180
ccattttctc tatcagaagg tctgagcaga gttggggcac gctcatgtcc tgatacactc 12240
cttgtcttcc tgaagatcta acttctgacc cagaaagatg gctaaggtgg tgaagtgttt 12300
gacatgaaga cttggtctta agaactggag caggggaaaa aagtcggatg tggcagcatg 12360
tacccgaaat cccagaactg gggaggtaga gacggatgag tgcccggggc tagctggctg 12420
ctcagccagc ctagctgaat tgccaaattc caactcctat tgaaaaacct ttaccaaaca 12480
aacaaacaaa caaataataa caacaacaac aacaacaaac taccccatac aaggtgggcg 12540
gctcttggct cttgaggaat gactcaccca aacccaaagc ttgccacagc tgttctctgg 12600
cctaaatggg gtgggggtgg ggcagagaca gagacagaga gagacatgac ttcctgggct 12660
gggctgtgtg ctctaggcca ccaggaactt tcctgtcttg ctctctgtct ggcacagcca 12720
gagcaccagc acccagcagg tgcacacacc tccctccgtg cttcttgagc aaacacaggt 12780
gccttggtct gtctattgaa ccggagtaag ttcttgcaga tgtatgcatg gaaacaacat 12840
tgtcctggtt ttatttctac tgttgtgata aaaaccgggg aactccagga agcagctgag 12900
gcagaggcaa atgcaaggaa tgctgcctcc tagcttgctc cccatggctt gccgggcctg 12960
ctttctgcaa gcccttctct ccccattggc atgcctgaca tgaacagcgt ttgaaatgct 13020
ctcaaatgtc actttcaaag aaggcttctc tgatcttgct aactaaatca gaccatgttt 13080
caccgtgcat tatctttctg ctgtctgtct gtctgtctgt ctgtctatct gtctatcatc 13140
tatcaatcat ctatctatct atcttctatt tatctaccta tcattcaatc atctatcttc 13200
taactagtta tcatttattt atttgtttac ttactttttt tatttgagac agtatttctc 13260
tgagtgacag ccttggctgt cctggaaccc attctgtaac caggctgtcc tcaaactcac 13320
agagatccaa ctgcctctgc ctctctggtg ctggggttaa agacgtgcac caccaacgcc 13380
ccgctctatc atctatttat gtacttatta ttcagtcatt atctatcctc taactatcca 13440
tcatctgtct atccatcatc tatctatcta tctatctatc tatctatcta tctatcatcc 13500
atctataatc aattg 13515

<211> 14553

<212> DNA

<213> Mus musculus

<400> 6 (SEQ ID NO: 16)

cttgaagaac acatgttttc caagagggag cacccatgtt ggaatgacaa tgtagttagt 60
gctcctctcc tgtaggttag tgctcctttg ctataggtaa gtgctcctct cctataggtc 120
agtgctcctc tcctataggt tagtgctcct ctcctatagg ttagtgctcc tctcctacag 180
gttagtgctc ctctgctcta ggttagtcct gctctcctat agtacctaga gagctagggc 240
aaatgggcta ggcccgaagt gcagagacaa acagctatgg aagactgggt aagcacttcc 300
aagctacgaa agagcagtgt gaagggtcag ggcttgtgca gttagtaggg gagatcttcc 360
agttgaagaa acagaagaac tgagagccac tgggtatcat cctcctgcgc catgccttcc 420
tggatactgc catgctccca ccttgatgat aatggaatga acctctgaac ctgtaagcca 480
gccccaatga aatattgttt ttatgagagt tgccttggtc atgctgtctg ttcacagcag 540
taaaacccta aataaggcag aagttggtac cagtattgct gtgatagacc tgaccatgct 600
ttcctttgaa agaatgtgga tttggtgact ttggatttgc aacacagtgg aatgctttaa 660
atggagatta atgggtcatc aattcctagt aggaatatgg aagactttgt tgctgggagt 720
atttgaactg tgttgacctg gcctaagaga tttcaaagga gaagaatttc agaatgtggc 780
ataaagacag tttttgtggt attttggtga agaatgtggc tactttttgc ccttgtctga 840
aaagtctgcc tgagactaaa gtgaagagaa tcagattaat tgcattgaca agggaagttt 900
gtggctgcgc tatctggaaa cttacagcca gcctcttgga cctcgggtga cttacgcaaa 960
tactcaggga cagagatgct tgactctgta ctgatgagtt gtcttggatg caaatatggg 1020
ctcttcattt gactacatgt cacgatgagt caggagctgc tctctccaga gtgtgacaaa 1080
gcgaggggat gctgacggta gctgttctag ctttgaaggt aagcctgcac ttatgctaaa 1140
gtcacacata cacgagccgg gtggagaacc tgtctgtgtg gagacacctt tcattacctg 1200
tggcatccag cctctcaagc ttggactgcc tgtgtgctcc tggactctgg aggtcccact 1260
gctctgtcct ctgctgctta tgatactgac attttaaaag aatccagtgg ttcccccctg 1320
tactcggtgt ctacttctac ctggatgttc ctcatttatg ttctgtgaca cttctctgtg 1380
actctgctgc attcctgggt gacatgtgga caccctgtcc ctttgcagac catgatgtca 1440
ctgtcactag tggaatcaga tgccccaagt gttgtcctgt gtttgggaac gtgacaggca 1500
gtacagaagc agaagaggaa gggtgaaaac ggaaatgtca cagcagcatc tgatgtgtgc 1560
ctcagtcacg catgctgctg attggaacta ctcagcatga gagagggcca tggtgaatac 1620
acaaccctat acacactgtg tccatttctc tctctctctt acacagagag agagggagga 1680
gggggagggg gaggcggagg gggaggggga gggagaggga gtgggagagg gagagggaga 1740
gggagaggga gagggagagg gagagggaga gggagagttt aatgtctgtg aagagatacc 1800
atgaccaaag caactcttat aaaggacaac atttaattgg ggctggctta caggttcaga 1860
aattcagtcc attctcacca tggtgggaag catgcaggta gatgtggtgc tggaggaacc 1920
aagagttcta tatcctgatc tgaaggcagc caggagaaga ctgcctcttc tgcacagggc 1980
agagcttgag catagaacat caaagccctt ccccacactt cctccaacaa ggtcatacat 2040
acttcaacaa agacacacct cctaacggtg ccactccctg tggaccaacc atttaaacgc 2100
atgagtctat gagggtcaaa gctcttcaaa ccaccacact catgtacaca cacacacaca 2160
cacacacaca ctctcataca cacacacaca cacactcaca cacacacaca cacacacaca 2220
cacacacaca ccacacacac acacacacac agagttctat tttgcactgt ttcactgtca 2280
caaggttcta cttatctcag acacactgcc aggaattgtg tgggaagact ttcagtttct 2340
ttgggttcac atggacttag cagttcttgg tgatcctgaa agatttctgc agaaagaagc 2400
caaagtgttg agcccaaggc ctggccacac attagtcctg tctagatgaa caggggttta 2460
aaaataaggg ggcatcaagg tgaagccagc aggggctgac ttagagagga gacccaccca 2520
agccaactgc tcgaagtcaa aagcgatgaa tccccatatc cagctgtgcc cggtgctgtc 2580
ttgctacatc tttagtaaat gttcttttag ttgtatgcgt atgaatattt tgcttgcata 2640
tatttgtgta caccataggt gttcctaggg cctatggagg ccagaagagg gcatcagatc 2700
ctttggaact ggaattatag acacttgtta cccatagagt agattgtggg aaatgagcct 2760
ttagtcttcg agagcggcca gtgctcttaa cctttggtcg tttctccagg tctttgagac 2820
tttattttct tggacatcag gacaggatcc agggctttga gcttgtttct tcagccagct 2880
ttcttttcat gtatattaaa ttttatgtta ttttgctttc tttttcccca agacagaatc 2940
acactctata tagctcaggc tgggtttgaa ttcagtttcc ctgtctcagt ctaccgggta 3000
atatgattac agatgtgagt ctgactttgg tatcaaagtc cccagccctt ctggatatgt 3060
gttttaagga tatcagatat atccttgatt tgctttgaat tttcttttta gttacaacat 3120
aattagttcc gtgtcacctg aatatgtgta tgtcacctac atagtcttcc ttcttctctt 3180
cttccctctc ccaccttccc aggtacctgt ctgtcttcat atccttgtgc tgagagtctt 3240
gttgagggag atgatgaccg agacagagcc actggggaag ggagatgggc tagtgcaggt 3300
cttcagagag gagctcgtga atattgtagc ccctttagtc cctggcatgt cctcttgtat 3360
agccaccgcc atgctgtggc ctggcagaag tgaataagtt gtccagctgt tgacaggcct 3420
gccctccaga cccagtctga tcccaagaaa gggcatctgt gtctgtctct gaggccgtaa 3480
gtgctgcctg gttgtctcca gcttgacttg acactccctc cttaataaga gtaccacaga 3540
acagggtctg cagagtccct gggccaggtc cctgtgctgt cctggaatgc caggcgtgaa 3600
tttcctgtga agtaggactt tgctcgccaa gctcccacgg cttgcccttc agatagccag 3660
aattatctgg taccctgcat tgccgttcaa tacgcagagt atcactggaa gcgcgcgcgc 3720
gcacacacac acacacacac acacacacac acacacacac acacgcccac tccatcttta 3780
aaccccaccc cccagcaacg gcggtgtaaa cactctccat caggaagctg aaacgcagtt 3840
gccctctgct ggggagatga aggcagcttg ctgggggcga ggaccgtgct agcaaccttc 3900
cctggtgcac acgggctctg gtgcatgacg ggaacggaaa cgcggaacta aagtcagtcc 3960
tgcttttttt tttttttttt tttttttttt tttttttttt tttttttttt ggcgttggtg 4020
gtggactgag tgacaatcag tgaaatcact taggttgttt ttctcttctt cgttgggttt 4080
gatagacggt gggagagggt cagaggagaa ggggagggat ggggagagag ggaggaggga 4140
ggggcgggag gcggggggcg aggaaaacgt gctaacttct ccaatcctac aagacaaagg 4200
tttggagaaa gccgcactga gtgacccagc agaaggaatc caggaatgtc cgctggaatc 4260
tgactgttga ttccagcgcc atgcagagaa tctaggctgg taggaacatt ctttgtccta 4320
tccgacataa taactccaac caacacggaa aagaaaggct atacaagtga agaaatggca 4380
ttttcacttt catgactata caatcacttc caggtagtaa cacgtgtcta gcacagcggt 4440
tctcaacctg ggggtcacga tcccccactt ttctgcatat cagacatttt tacgttgtta 4500
ttcataacag tagcaaaatt gcagctatga agtaacaatg aaatgcattt atggtgcgtg 4560
tgtgtgtgtg tgggggggta tcaccttaac atttactgta agaaggttga gaatactgct 4620
ccagcagcta gtgtgttgga cttaggttct gggtatatta ttagcaatag ccaaccagaa 4680
tccccaccca ccacagcatt gaggccccat gcagggcttg ctgggagagg cactgataag 4740
acttctttat gtatttattt agagacgaat actcattagg taggccaagc tagcgtcaaa 4800
ctcatggcaa ttctcctcct ccagtttcct aagtactgga ctcaggagtg tgttgccatc 4860
atatacagta aggatttatt gactgaagaa aatctcaagt ggctttggtt aatccctact 4920
acgccagagg ctgaggcagg aggcgcgcaa ggtcaaggct tgcctgggct acatatagag 4980
tgagctcaat tttgacactt ggtgcggtgt tagtagtaat agtaaagatg aaggtgtggc 5040
tcaggtgggg ccggtgattg gacacacttg gggtctcctg gtccatctgc agctgtgcaa 5100
caggaagagc ggagaatgag aggaaagaga gaaaagacag aatgagagag agggaggaag 5160
agagaaaaag gaaaagagag aggaaaggaa aaaggaaaat gaggaaagcg agaaagaaga 5220
aatgagaaag aggaaaggga gaaagaaatg agagagagaa aagaaaagac agaatgcgag 5280
agagggagga agagagaaaa aggaaaagag agaggaaagg aaaaaggaaa atgaggaaag 5340
cgagaaagaa gaaatgagaa agaggaaagg gagaaagaaa tgagagagag aaaagaaaag 5400
acagaatgcg agagagggag gaagagagaa aaaggaaaag agagaggaag ggaaaaagga 5460
aaatgaggaa agcgagaaag aagaaatgag aaagaggaaa gggagaaaga aatgagagag 5520
agaaaagaaa agacagaatg cgagagaggg aggaagagag aaaaaggaaa agagagagga 5580
agggaaaaag gaaaatgagg aaagcgagaa agaagaaatg agaaagagga aagggagaaa 5640
gaaatgagag agagaaaaga aaagacagaa tgcgagagag ggaggaagag agaaaaagga 5700
aaagagagag gaagggaaaa tggaaaatga ggaaagcgag aaagaagaaa tgagaaagag 5760
gaaagggaga aagaaatgag cgagataaaa gacagaattt gagagaggga ggaagaaata 5820
ggaaaagaga ggaaaggatg gagaaaagag agaaagaaag agagatgaaa gagagaaagg 5880
agaaatgaaa tgagagagag agagagacac aaagagccag agagagaaga aaaaagggga 5940
aagagaaaga gaaagaggaa ggctcctctt ggacacatct tcctttatct ttccctgggg 6000
accgccaaag cctggtggca tactgtacat tctgtacact gttcattcaa aacaggctct 6060
gtcttaaaga tggtctgagc ggtcagaaaa gggtattgtt aacttgtttg caaaactgcc 6120
tcaggagagt gctgagtgcg tgaaagttgc tgcccgttaa ggagaagtct ctactacttg 6180
tgatctcacc atcgaaaatt tctttaattg tctcctggtg ttctgggttt tgcagttttg 6240
tttctaagga tacattcttg ggtgatgtca caaagtcccc aaagacacgg tggagctgtg 6300
ttagatgggg aaagacagtc tgctgaggat ttatctggaa ctgtcagaag gaaaagaagg 6360
taaatggggc acttgggaaa gtggcctcta gtttgacttc tggcttagca aaggttgtgg 6420
ggagataagg catacacagt agttagcagg aggcaacagg gtcctgggag gacgcgaggc 6480
agaaggagag gctgggctga cagcatgcaa tcattgcata gtctccaaag gagattgcaa 6540
catggctgag ttttcagagg tcctacagag cccgtggtag agattctgtg ggttctgaga 6600
caacttgact ttagccagat ggtatttgag taatctggga gagagaaaac agctacagca 6660
aacagggcca catttagtga cgaaactctc actttgactg ttgagtcatt tgcagtgggc 6720
cctgaggtca ggctggccct cagctcaaaa acaagcgagg aactgaagca attactcaga 6780
taatccacag ccacagccac tggaaagggc cacatcccca gagacagcac agcaggggtg 6840
ggggtggggc tatgagaaag ttagtgattg tagcagttat ctagaatgtg cggagcagag 6900
gaggttacac aaaaacctag aatgtcattc aatgtgggaa accgagaggc tcccaagccc 6960
taaaaggaac agtttgcttt cagccaaaat ggaaataaaa tttggggctt aaatctggca 7020
aatgattcag accttctgtg taggtgtctt taaatgcaca gcagattgat tttcatgttg 7080
gagtttattt gaactaaaag acagaaatgg tgaaaagcac acctgaagaa attgagatgc 7140
tatgaataaa atcatttact tacagctatc acttaattag tacctccttc caccttgctg 7200
atttattggg ctagtcaagg aagaaaagat cttccctcct ccttctctcc tcctccccct 7260
cctctcctcc tcccctcccc tccttgacct tcctctcctc cttttccctc ctccccctct 7320
tcttctcttc accccctcct cccctcccct cctctgtact cctccccttt cctcccaatc 7380
tcttttttct cccccttctt ctctttctcc cccctcctct tccctcctct tcctccctcc 7440
ctccctcctc ctcctcatcc tcctcttcct cttcatcctc ttctccttcc tccctctcct 7500
cctcctcctt ttccagccct acctaccttc cctttcttct tcatttattc aaagtagctt 7560
tgaacagcac tactcggttt agttgtgtat aaaaggaaaa tgcaggtcca agcagcttgg 7620
ggaagattgc tttttgctct ctggaggcag atgatgacag ttcaagatca ttccttttgc 7680
tccatgtcac aggaaggggg acatgccgaa tctaccagtt tgcagccacc tacacaggat 7740
ccaccttcac ttctaaggaa atgtttggga agctacctac caaccacttc tggcatctca 7800
tgggctagag gactcttaaa tggcactctt atttgtttaa taaaggaggt tgtgacgtgt 7860
agttttaaat cccttccaca caacaattgc tactctctga ccaaaaaaga agggagacag 7920
gatacggcta ggtgtctagt agactttacc actttgaaaa gccttaatat aaatcaggta 7980
gatacatctt tttaacttat tcttgtaaag acaaaaacaa aactttattt ttatttgtgt 8040
gtatgcttgt gtgtgtgtgc ctgtgtgtat accacatgtc gctggtgccg gagaacacca 8100
gaagagggga cctgatctcc tggagctaaa gctatccatg gttctgagct gcctgatgtg 8160
ggtgctggga acagaactct ggtcttctgc aagagcaaca agcctcctct taactacgaa 8220
tctcctcccc atccccccaa atacatttaa ttattcattt tagcagcttt atttcgtaac 8280
tacttatcac agcataaaac aaggatttta tatatattac atgcaatcga ggataagagt 8340
tgaggggaga tgcgtgtgct ccttctgggt gtctgtgctt ttgaagaatg taagcagtgc 8400
acaagggacc gaggcgtgcc tgtctgccag gagctgtctt cttcccttgg actctgagct 8460
gagtgcagtg ctccgaagaa gtaaaagacg acctcatgaa gcaatgtctt caacccaaac 8520
atgctgtcca gacaaagtcc agcttcatta gtgctctgag gagagactta ctgagcctca 8580
ggaaagcccc cctcagcatg gcgaaagtcc actttgattg aagtgactcg aaagccatgg 8640
cagtgcggcg gcggccgcgt ggagcttgtg ctcgagtcgg aagcggcatc tttgtcaggc 8700
ggctgtgatt agcacgggga ggcaggactg gagtgaagga agagttgggg gcggggctta 8760
gcgctctggt ctcctaagct gtagtcagcg cctcaagatt tgtaacctgc cttctgcctt 8820
cccagccagg cagtcaagtg gctccaagct gaagactgca aagtgcccct aaccttttgg 8880
ttatagcgag gctgaagaca ccgtgctctt tcatgaaagc cggatgtctg aaatccgatt 8940
tgataaatat ggataaaacg tataacgctc gatcaatcga atcgaaggag ctcacgattg 9000
gcaccacggc tttggggaca acagagtact gactcgttgg gaggacttgg atacttcccc 9060
tcctcttcca tctcttcccc tttcctcact tcctcctcct tccttctcca ttttctccct 9120
cttcactgtt tcttactatt tttacaaaag attttattta tttatttatt tatttattta 9180
tttatttatt tatttattta tttatttaat gtatgcgagt acactgtagc tgtcttcaga 9240
cacaccagaa gagggcgtca agttccatta gagatggttt cgagccacca tgtggttgct 9300
ggggcctctg gaaggaccgc cagtgctctt aacccctgag ccatttctcc agtacccttc 9360
tcaccgtttc tcttcaatct tcttcctctt ccttctccac tttccttgtc ttcttggttt 9420
cattatcttt ctccctttct tcctcttctc cccttcttcc tcctccactg tagttttcct 9480
tccctactct tttcctgcct ccctcctcct cccctctcat tccccctcct ctttcctcct 9540
tctccctcct cctccttcct tctccctctc ccctctcccc tctcccttct cccttctccc 9600
cctcctcttc ctctttctcc ttctccaccc ctcctgtcac agtatcaatg gcaagggtgt 9660
tctagaatgg aggagtgtcc cctaggcact aacgaaagcc agttaggatg ctctgagacg 9720
ggtacaattc agggagggcc gtggggatgg aagggttgtg ctgcgattca ttctggagca 9780
acccccaggc agaatcatga ggttggttcc ggattcgcag ggcacaattc agaagaggaa 9840
ggtttcagga aggacgagtt tgtctgagat aggagttaca tctgatgtct tggcagcaga 9900
gccactgtac aagcgtgctt tattaaccac gtgggattaa atcttctttt aaatttattt 9960
tcaactctta aggaaacgtg aactttcaca ttcaaattta gacttgcagc tcttatgggg 10020
aaaaaaaggg gatcttaaga atattaagca taggcggctg gagagatggc tcagcggtta 10080
agagcactct ctgctctccc agaggtcctg agttcaattc ctagcaacca cataatagtt 10140
aacaacagtc tttaatgaat tctaatgccc tcttctggtg tgtctgaaga cagttacagt 10200
gtactcatat aaataaaata aagaaattta aaaaaatgaa tattaggcat agattcctgg 10260
atcctaagaa agccatcaga gctggagcca tgtgtgggat cctgcttggt gctggagggg 10320
cagagttcat gcccccgggg tttttactta ttatcacatt ttcatcgttg ttttgaaaca 10380
gggtcttgtg tggtccaggc tggccttgaa ctcatctttc agcctctacc tcacaggttc 10440
tgggattact tggttcctaa aagtatctcc gtcaagctcc ctggtgttat ggctgtgcca 10500
accaggaggg tctatacact cgctcaggta gagggagaag atccgaatct ctgacaggga 10560
ctgctgcctc tcggggcaaa tggagtgaag gacagcggca gaaggattta ggaaagatgg 10620
acgggagagt ggaaatgctg cagaagccag aaaacaaagc aggaagcctg ctgtccagtg 10680
gggctcaaga gcggagggat gcgagggggc tgcgcaggaa catttagcgt ctgcgtctat 10740
gggggtaggg gcggggtgcc agcacctagt cacctgaagg ggaaatgctt gcccagggag 10800
caggtctcag tagctgacct agagaaagga gcggccccta cagaggagac acgggtcact 10860
gtttgttaaa gtgaaggaga aataaatatt ctttcaaaga atcttaggtg agcccagttc 10920
atctgcgctg tggaggcctg gggaacagtt aaaaagaccc tgacacacac ccaaggcaaa 10980
caagcaacac acggctcctt ccgtaagggt ccatgattct ctgaagaatc agccccggaa 11040
tcagccccgg aatcaggtag tccgtaaaca caatgagtgt tttactctgc agaagtccag 11100
cctgctggcg tctcccatta ccaaaataga gggatagtca cgtgagctca ccggctcgat 11160
ttaaggcacg tggttttcca gggtagatga gctttggctt ctggaaccat tatggggcac 11220
gaaggatgga gccaggattt tttttttttt tttttttttc tattagcaat tgatttgctt 11280
gggcttggct ggacttgccc agttcttagg cccagtcttc ttaactgccg atctgaagtc 11340
tgtcatggag tcagcctagc cttctcactt cccttcagct cgaataggaa gaggaggtgc 11400
acaccagatg gtctgagagc agggataaat ggtgtgcctt tgtctttcag tatttcgtta 11460
ttttaagtag gaagatgctt ttctgtatta cattgcttgt gaaaccggaa gttgattcgg 11520
ggcacaggac aatggatttg gtgttttgca aggactgttt cagaagagag aggagtggaa 11580
gggtggttag agtgaggagt ggggtgggac gggatggggg aagagaagga agggccagac 11640
aggctaggta gggctgagag gaggcggtgg gaacttcttg agttagcgca gcagtaaact 11700
tggatgtgcg tgtatctttg tgatatatga cccggagccg tgtagctggc tccgatagta 11760
ctgctaatgt cagtgtcggg gggggggggt cccatactgt tccacagggg ctgcacattc 11820
ccatcgagag caggagggct cctctctcca tacatcctcg ccagcattcc ttgttgtttc 11880
tgtgatgaca gggggtggga tgaaatctct ctgttggttt gagagaccgt gaagaagctc 11940
aaccccagga cattttgcag tcttggaagg cagtgcctcc atgtggagcc gtggagccca 12000
tctctgagtc caggtcactc ttgcagttcg cactcagctc ttcagatgca ggagagacgt 12060
tggtgggaaa gcaagattgt ttgcttgttg agatagacac attctccaca caaaggctca 12120
cgtggggcaa aggctgattg acgtacagcg ttcaggaacg cctgtggtag agctatgatt 12180
agctgtctcc atctatgaag cagacaaaga gttataaaaa aaatcaatgt tttcaaattg 12240
tcaaactttt aacccgacag caagcgctct gtccctgggc taatccctag ccctggtttc 12300
ttgagatggg gtcttttgtg cactagactg gcctagaact cacgatctta gtgttccagc 12360
ctcccagctg ctgggatgag ccgctataac cagtctgcct gccttcctaa attttaagtg 12420
atgggaagtg ggggagaata cagtttaaag tatgcagatc tgagagcagg aacctggcaa 12480
agccaagggg ccggagttac aggcggctaa catgggtgct gggaactgac ccaggtcctt 12540
gagaggagca gtgtgtactc ttgaccaaac aggtccgtct ctccagtccc cgtagtatta 12600
aaaataggta ctacgggcat ggtggtgcac acctttaatc ccagcactag ggaggcagag 12660
gcaggtggat ttctgagttt gaggccagcc tggtctacaa aatgagttcc aggacagcca 12720
cggctataca gagaaaccct gtcttgaaaa caaaacaaca acaaaatagg tactacaaag 12780
cgatgtaatt gtgctcaaac atgcaaaccg aggggactgt atgcataaga aagagaaaga 12840
cggccacact ggttctatct gggtgacagg aaatcagtat ttttattttt cacattcatt 12900
tttttgttgt tgttgttgac acagtgattt ttctatcaaa aacattattt cttttatagt 12960
tcccctgagg agctgttttt aaagccgtgc tttgaaaaac cattgaagga gcagaggcag 13020
ggagactcct gtgtggcagt cggtgaagca ggccctctgc aggcaggctg gccctggact 13080
tgggagtctc tttccctccc tcctgtgctc aaatagcaaa tgtcaggctt caatgtagct 13140
agaaggttct agaatgatta agtttccaag gctgaagagc ttccctgttt gcctttcact 13200
tccctggaga ggtcgttgtg tgttccggag tctgcaaggt gcctttggtg atgcgggtgg 13260
ttcatctcgg gagattccgc ctggaggacc caagttcaag ccctgcctga gctacagagt 13320
gactttcagg tcttctgcgc aattcagtga gacccagtct acaaataaaa agtaaaaaga 13380
aggctgtgga tggaactcgg tggtagagtt ctgggtttac tccctagagg aggggagaag 13440
gaggaggagg gaggaggaag aggaagaaag aagaagagaa gggaagagga gaaggaaggg 13500
agggaagggg ctgacaagaa gagagaagag ggagggaggg gagggaaagg aaggggaaag 13560
gaagggaggg aaggggctga caagaagaga gaagagggag ggaggggagg gaaaggaagg 13620
ggaaagaaga gaagggtaag aagaaactgt tccaatggtc tgggccacag agtgatggcc 13680
ttttgtggtg atcagctgta atccttgatt tgacacaacc tagaatctgg gaagcgagtt 13740
tctgtgaagg agcattcaca ctggctggcc tgtgggcgtg catgtgggag actgtcataa 13800
ttaggttcat taatacagga agtcccagcc cactacaaat ggcttcgttc catacccaag 13860
agatgctaac tgtagacggt tggagaaagc aagcaagctg tggatacccc acgctctttc 13920
acctcggctc ctggggggtg ggtgcactgt gtctcttggt attttaaagt cctgccttga 13980
cgtccctgct gtgacagact gtaactggaa ttgtgagctt tagtccttta gttttctacg 14040
ttggtttttc tcaggatatt ttatcgcagt aacagaaaca agaccaggac acttgatctc 14100
ctctgatcaa cactgaagag ttacaaaaca ggctgaggaa acaaactttc ttctccctct 14160
cccccttctg tccctcccct tccttctcgc tccctccctt gccccctctc tccctgtctc 14220
tgtctctgtc tctgtctctg tctctgtctc tgtctctgcc tctcccctcc cctcccctcc 14280
ctctgtctct gtctctgtct ctgtctctgt ctctgtctct gtctctgtcc ctttctcctc 14340
tatctcctaa atggctggag gccatgctag ctcaatgttg aactttgaac acgtatttag 14400
gaaatctttg ttcttaacag ttctgaagtg ctgaagtggt ggtttagtct ctcggcctga 14460
caagctcact tcctctcact ctgtcttaat gaccaaatct gccatttccc taaaacagca 14520
caggctccag ctccaggttg ctccggagcg gag 14553

### CHO Stable Site 2 Sequences - U.S. Patent No. 9,816,110

<211> 4001

<212> DNA

<213> Cricetulus griseus

<400> 1 (SEQ ID NO: 17)

ccaagatgcc catcaactga ttaatagatg ataaaattat tgtacatttc agtgtaatat 60
tattcagttt ttaagaaaaa tgaaattatg taataagcat gtaaatggat atatcttgaa 120
acaaccattc cccattatat tacctaaaca ttgaaagtcc aaaatcatat gatcttttta 180
gtggatctac taatcttttg ctatatgtat tttattgaac tacccatgga tgtgagataa 240
ttggtaacaa cagcacatgg gagagcatgg gatcattcaa ggaagattag agagaatgca 300
ttttttagga gataatggag gagcaataga aaggattaaa tgaggttact gatgaaagtg 360
atggttagag aaggcaatat gaggagggat aactagcact tagggccttt tgaaaaagac 420
atagagaaaa tactattgta gaaacttcct ataattggtg tatagttata tacaccaaag 480
agctcagatg gagttaccct ataatggaaa tattaactac tttttatcac tgtgataaaa 540
catcctgaac agagcaacat agattgggaa gcatttactt tggcttacag ttctaacggg 600
ataaaaattc atgatgaaag aatgaatatg tcagcaaaca gcagtagcaa tggcctgaga 660
agcaggtgag agctcacatc ttgaagtgta agaatgtagc agagagaaca aactgcaaat 720
gaccagaaaa tgcttttgga tcagagccca tacccctctg actgacttct ccagaaattc 780
tgaacaaata aaactcccca aacagagcca taactgaagg tccagtgtct gagactacta 840
ggggtatttc ttattcaaac cactacaatg gggtgggggg agcaatcctc caagtaggca 900
ctacacacag acaaataaaa actctagtaa ctggaatgga ttgacttatt tgaattactt 960
gccagtggag ctacatagag cacaattatt gtatttaaat taccctttat gatcttacaa 1020
aacttgacag taagatcata ttgctaaaga aaccacatat ttgaatcagg gaacatggtg 1080
atatctagtt gttcttcaac tggaaacttc atgctttctg cccagcattc atgttgctgg 1140
aaagagcaat gtacactacc agtgtagaaa ttaaatcatc aatcttatca agatgtggat 1200
cctataagtt acaataaaaa ttagcctgat aagatatccc caccagaaga atattcacat 1260
aaatgctatg ggagcaacaa gctattttct aaattagctt taatcctatt ctacaagaga 1320
gaatccatat ctagaatagt tatagggatc aagaacccat ggcttgattg gtcataggcc 1380
caatgggaga tcctaatatt attgttctac aaaatgaaaa taactcctaa tgacttgttg 1440
ctgcagtaat aagttagtat gttgctcaac tctcacaaga gaagttttgt cttacaataa 1500
atggcaatta aagcagcccc acaagattta tatcataccg atctcctcat ggcctatgca 1560
tctagaagct aggaaacaaa gaggacccta agagagacat acatggtccc cctggagaag 1620
gggaaggggg caagacctcc aaagctaatt gggagcatgg gggaggggag agggagttag 1680
aagaaagaga aggggataaa aggagggaga ggaggacaag agagagaagg aagatctagt 1740
caagagaaga tagaggagag caagaaaaga gataccatag tagagggagc cttgtatgtt 1800
taaatagaaa actggcacta gggaattgtc caaagatcca caaggtccaa ctaataatct 1860
aagcaatagt cgagaggcta ccttaaaagc ctttctctga taatgagatt gatgactacc 1920
ttatatacca tcctagagcc ttcatccagt agctgatgga agcagaagca gacatctaca 1980
gctaaacact gagctagttg cagacaggga ggagtgatga gcaaagtcaa gaccaggctg 2040
gagaaacaca cagaaacagc agacctgaaa aaaatgttgc acatggaccc cagactgata 2100
gctgggagtc cagcatagga cttttctaga aaccctgaat gaggatatca gtttggaggt 2160
ctggttaatc tatggggaca ctggtagtgg atcaatattt atccctagtt catgactgga 2220
atttgggtac ccattccaca tggaggaatt ctctgtcagc ctagacacat gggggaggtt 2280
ctaggtcctg ctccaaataa tgtgttagac tttgaagaac tcccttgaga agactcaccc 2340
tccctgggga gcagaaaggg gatgggatga gggttggtga gggacaggag aggaggggag 2400
ggtgagggaa ctgggattga caagtaaatg atgcttgttt ctaatttaaa tgaataaagg 2460
aaaagtaaaa gaagaaaaga aaacaggcca aaagattata aaagacagag gtggtgggtg 2520
actataaaga aacactatta tctaaataaa aacatgtcag aagcacacat gaacttatag 2580
tgtttatgaa agtatgtata ataactacat aatctcaagc caagaaaaaa atatcatctt 2640
tcagtgatga aggtgatttt atttctccca gaattaaagc caaagaccta atgaaagtaa 2700
ttatcttcaa aaggttgaaa atacatactt tgcaatacac agatctgcct agaaatctca 2760
tgttcacaat acacatgatg ctcaattgaa ttccattcaa tgttacagtt tagataaaca 2820
gtttgtagat aaactcacaa tgtatcattt ctttttattt tttgaccaaa cagcttctca 2880
tctgttattc agaataattc ctcgatggca ggatatccat cccaattggg ggaaggggag 2940
aatttgaaga aaacctagac cacatacata tttgccattg ggaaacaaag tctaaaatga 3000
tgttgttcac atcttctcta ctagtcctct ccccgtccca aagaaccttg gtatatgtgc 3060
ctcattttac agagagagga aagcaggaac tgagcatccc ttacttgcca tcctcaaccc 3120
aaaatttgca tcattgctca gctctgccct tctcatatga cagttacaag tcaaggcttc 3180
caaagtccct ctgtcatgtt tggtgtcaat agtttataca gatgacttca tgtcttcata 3240
tctaatgtct tatatagatt aatattaaac aatgttattt ctctaaccac attttaaatt 3300
aatttaaaaa tccattaatt gtgtctataa aatgcagaca gagtgctgag acacaatata 3360
agcctgatga tctgaatttg aaactcacac ccaccacatg gagaatcaac ttccaaaaat 3420
tttcctatta cttccacact tacaccattg tacaaacaca ataataatga acaaaatgaa 3480
atgaaataaa aaattaagtc tctgtaggta atgctactgt gcagcaaaag taaaaatggc 3540
agcttaagct tgctttatgg ttacacttta ccatcttcca ttaattataa ggacttcaat 3600
catggcagaa ctatgctgtt attgtctcag tgtaacctaa ccaggtgttc cagatgttct 3660
taatgtggac acctaaacta tttgatattt gggttaagat ctttccctct ttcagaagaa 3720
acctcaggac agagggaatc ttgtctttta attttgagtc tgtagacttt ttccatttca 3780
aatatacatg aaacaagtga tgaagaaaat taatcaaaag gtgggaattg caatgatatt 3840
aggttcaata ttaagcttca atattatcat ggaatcgcct gttatacact gagtgtttgg 3900
caataaggga tttttagaag aaggagtttt tattctcaac aggttcctta agtttagctc 3960
aaataaatct aagcaatcca ctctagaatt aaatagtttc c 4001

<211> 14931

<212> DNA

<213> Cricetulus griseus

<220>

<221> misc feature

<222> (2176)..(2239)

<223> n is a, c, g, t or nucleotide is missing

<400> 4

(SEQ ID NO:18)
catgtacact tatgcaagta tgatatggcc caacacagta ttttacacca atttttatct 60
ataaaatata catgtacatc aaaatatatt attaataata acatcattat tctttctttc 120
caagtaataa acacatacac tgaaattttg gttcttgtgg ataattttaa tgaaacagga 180
aatgcaaatt tatcttagca tgtttacttc actttctttg catagataac cagtaatcac 240
attgatggat catgtagtga aatgtatttt taggtatcta aggaattttg gcttcgtttt 300
gtgcttgttg acactgaatt ctattcctaa caacagtgtg taaggattct gtctgatttc 360
ttttaccagt atttgtccat ttgcattttc tttattattc atggctgctg ttctagaaag 420
tggaaggtag tgtgtcaagt ctgtttaaca tgtttccctg atgatcagtg tcttaacacc 480
tctctgagta catgttggcc aatgtcgttt ctagacccat ctattcttgc ttgacttatc 540
ctggtacatg cctgccaaga aatttctcct catcctttct gtctcttcac tgatttactt 600
gatgtgtgga tttcacattg atcatatgga aatagaagat acaattttct ttattcacag 660
tttggaagac tttcaatctc atagatcatc attatttttt gctactgttc cctatgctat 720
ggtgaaattt ccatttgaat aattgcttaa acaattaaca agaaagaatc tatttttact 780
tgcaataact tccatttcag aacatttact acactgttac tatatccaaa aactagtttt 840
atatatcatg tgagaaatga ctaattcata atttggccat gacatttttt tcagaaacag 900
aaaaagtgac caatacatac acaatgctat aaatattaag acttcagcaa attaaatatt 960
tattcatgat atcacataaa attcatttat tatgttttat ttaaatgtgt ttttaaaaca 1020
gtggtatcac taaatattaa gttagatgtg tttatgtgct taatgaattt atattttaga 1080
atgttataag ttgtatatag tcaaatatgt aataaatttt attttttagg tctttctcat 1140
taaggtattt taattttggg tcccttttcc agagtgactc tagctcatga tgagttgaca 1200
taaaaactaa acagtacaaa atgtacattg cattcagtat tgcacttgat ctttgcactg 1260
aagtttgagt cagttcatac atttagtact tgggaagtac attaagctaa ctttcattgc 1320
tctggcaaaa tgctcgataa gataagagtc tattgtggaa agccatggca gcaggaaagt 1380
aagactgctg atgatgttta atccatagtc aagacgcaga aggagatgaa tgctggtatc 1440
caacattttt tgctgttcat tttctctaga accctagtcc ataaagatgt atgacttgca 1500
ttcaaaatgc gtccccttca gttgttcaac ttttctgtaa atatcctttc aggcatgtct 1560
agaagattgt ttcgcaaata cttctcaatc cattcaagtt gatagtgcag attaatcact 1620
gcagaataaa agcctgtaac ttggctcacg tgccaaggaa tatgcacact cctgacacat 1680
caataagtaa atcaaagtgt agcttttgcc tttaacattg ccagacttat gtaatgttct 1740
gcacgttctt cctccatcac tttttattct aatggtgttt ccttgacatt gaatcacgct 1800
gtggaagctg cttagaatta acattgaaat ctactgatat atttatgatg cagcaattta 1860
gatttactat tttacttaga attttttata attgagagaa tataatattt tcacagttat 1920
ctatctgctg taaatagagg attttaaaaa aaatctctat aacttttttt tacaacacac 1980
agtaaaatta agttaaaatt taataaagtc actatgttga tttcaaagtg tgctacgccc 2040
acggtggtca cgcaggtgta gcagaagatg ccactaaggt gggctaaggc cgatgggttg 2100
gggtctgcgc tccctggaga tgagccccag gcggttccct ggcaatcagc tgcgatcatg 2160
atgcccgatg agccannnnn nnnnnnnnnn nnnnnnnnnn nnnnnnnnnn nnnnnnnnnn 2220
nnnnnnnnnn nnnnnnnnnc tgggtgactt tatggaaaga atttgataga tttcatgatg 2280
tagaagaatt ttattaggct tattttacag gagactaaga ccctgggacc taaagatatc 2340
tgggtcctga gaatcaggaa atgggtagag acgtggttga tggtatgaga cagattttag 2400
agaactctta gatcatgggc aatgaccgca atctgatgct tagaatagat catctataaa 2460
caattatgct gttctttttc tttctgttgt atgatctgat gatgtagccc ccttgccaag 2520
ttccctgatc ccccttgcca agttccctga ttgtaacagt atataagcat tgcttgagag 2580
catattcaac tacattgagt gtgtctgtct gtcatttcct cgccgattcc tgatttctcc 2640
ttgagccttt tcccttgttc tccctcggtc ggtggtctcc acgagaggcg gtccgtggca 2700
aaagtgtata aatgttctaa aacatttgaa ctctaaaaca tgcaaaatga aaaattaaaa 2760
taaataaaca tgaaaattaa aatatattag ctgctaaaag ttaaacaata ctatataata 2820
ttttgttatt agaattcaaa atcacattag ttggatttaa tttgaacatt gcattctttc 2880
aataataatt tcaataaaaa aagtttcccc atgatagtag aaaataataa catatgtatc 2940
tatctattta tttaactaca catatatagc atttgtttca actaaaataa atgaatgagc 3000
aaagcaccta agtaattggt gtctattata tttatgaagc caatagtttc aaataaatta 3060
tcatgcataa ggaggtattg caaatgttaa accttttttg aaacagatat tcccagttac 3120
agaaattata atttctaatc tttcctataa gtagaatgat gataattaat ataggccatt 3180
tgtaaataat gttcagatta aaatattctc tatttcacta gagaagaatg atattaaatg 3240
tattatattt tatttcccat tttgtttgca ccactattct atatccctca gcagtttaaa 3300
tttgtttcac catatgtgtg tgtgtttgta tcttaaatat ggcactaaaa ttagaataat 3360
ttaatataaa tctttaggag aaaagatatt gaattatttt atgttgatag gaaaatatct 3420
tttaattgtc caagaatact ttttcttcta ttttaggact gatcagaccc aggactaata 3480
ttttatatgt actaattcta tgtaccaaaa tatgttatta tctcatgaat tctgtctcaa 3540
tattgaggta ataaaaatag tccatcatga actttaaaat taaaataatg attaattaat 3600
ttttattcat attttgtttg tatgaatggt tatacatcac atgtgtgcct ggtgactgtg 3660
aatgtcagga gaaggtatga aagccactgg aattggaata agagataata tttgagatgt 3720
tatgtgggtg ctgagaatta gacgcaagcc atcttcaaga atagccagca tactatacca 3780
ctgagtaatc cattcatccc tcaataatta tctttgtaga cagtaaatat atttctaaac 3840
tataaatgac cagaaaaatt aatgtattat taatgaagac attcatctca tgtgacacac 3900
ttcacctgtc taaatcagta acactctctc cactaattaa gattttctaa gtgcatgaca 3960
cttactattt ctaaagctgt ccaatggggg ccagtcccca gtcagcaccc agtgagataa 4020
tccatgaatg catttatatc ttaggaaaaa ttcttatcta tgtagtattt agaacatttt 4080
catgtgaggg gataaacaag gaagcacaga tgctttctga tagaaacttt ctctttaatt 4140
catctagaaa aaaaaaacct ctcaggaaaa tctctcttgc tctcctccca atgctctatt 4200
cagcatcttc tccctactta attctagatc tttttctcta tgcctccttg ctgctgccct 4260
gctggctctg ctctatgcct ccccatgtca cttttctttg ctatctcacc gttaccttct 4320
ctgcctcact ctctgccttc ttctctgctt ctcacatggc caggctctgg acaattatag 4380
ttatatgtta cattctcata acacatgata tgtcacatag tttctctcag gctagggata 4440
tcacaatgac tggccaatga gcaagtggcc ttgcatgtag ctctaagttg gtgatggttc 4500
ccagacagta agtagccatt tggttgaaat ttgaggttgg gtagtacatg aagactgaat 4560
tttcttcaaa ctctggcctt gaaatagtaa aacaacacct atgaaaatga cgacctgtat 4620
ttgtctttag aggcaaccac atattgtctg cagggcctgc tttgaatttg ctctgaagtt 4680
agcttgtttg tgtaaaagga agaatcctat atcagcctga gaaatgtaaa atatcctagc 4740
atttcaagtc atcaaaatta tatggagagt ataaatcatc cttctgacta ttcatagtca 4800
tatttgtgtc caccaagtat aaaacacact accaaagggc tgtggaaaaa atcgccataa 4860
ctgttcttat tagggaggca tagcagtggt acctgaggaa gttacagcaa caaccagtca 4920
tccagtcaat aaccccatgg ctttgccact tggaggtacc caataatgtt tggctttgcc 4980
gagtaggact ccaacaaatt cagagggtca atttttaaat gctggttgtc actgctgaac 5040
agtcccattg ccctctgcat aattccacaa tggaaagctt tttacactga ttgccaatca 5100
ttaaacagcc tactcagcat aaacaggtat gatattattc tgcattttgt tacattacta 5160
gatgaattcc tatttcttcc tacaatagtg gaactgaaaa aagatacaca atcatactac 5220
ccctctacta atcttatgac ttatatcatt tcaattttca gaccataatg caaactattg 5280
accaaaacat gtgaagatga aaaatagaaa tgtagaataa tattacatat aaaaagaaaa 5340
ggcggactta ttttgtttta tttcttagca tgcatagcaa tacatgattt gaggtttata 5400
taataaaggg acaataaatc ttcaagaaac ttacccctac tgaattaaaa tattaaagaa 5460
ggtcacacat ttactcaaat atattagact actgggcaaa tagacatgaa aagtagagtt 5520
aatattgagg taggccttct gtgaaatgtc taaggaaatt atgtttcata cagtgtgtaa 5580
ccaagtggga atcatatcag aaagcagtca aaagcttata ttacaagtaa cagatgcttg 5640
gttatatgac ctcccagagc ttgactgtct atacacaaaa agtggtgtta ataaaactgt 5700
aatttgggct atgttttttt aaatggcttc accaacatga aaggaaggga atgagcatgt 5760
catggatgct tagagattat gcttccagca agaagaattg agctttggct cttattacag 5820
aaacatgaca aggtgtgagt tttatttatt agaaattata taatatttta agctggggac 5880
taaaaatttt attgaaacaa acaggcaagg gataggcatg tactagaagc aaaaatagga 5940
tgtcaatgct gtaatgttat tttttggacc aaaatagtat ttcctataga aatgacaatg 6000
atcttaggtt attattcttc ataaagatga caagttcaca agatatccta gttcattaaa 6060
atcgttttag tcatttaata gagtgctgtg atagattaca caaaggaaag cacttacgat 6120
gagaaataat gatatccaca attattttct taattcttag aaacattcta ttgttatatc 6180
tcaatctcag aagccactta ttgctttatt attgaaacat atgaaattgt aagttatata 6240
ttgtctatgg tgacatttca aagaacatgt gacgtacagt gtagcacaga taaagaacat 6300
aactgcagct gaatcagtaa ctaaacttac atacattaaa tctgccatgt tggcaacagt 6360
gtgtgcacta ccaaaggatg tactaatgct cacgacactc ccctatgtca ccctttgttc 6420
atcattacat cataggtcta ttttgtttgc ttttgaaatc tagaccaagt cttttgtgtc 6480
tttccaagca cagagctcat taatttacct catagacttg ttaaacttct tctggttcat 6540
caattgaata gaaatactca ctactaatta tgtgagaccc tgccagtacc atagcacatg 6600
gataattttt acataaaaca tgcatacaag taagattatt cagactgaac atgaatttta 6660
gagaaatcag gaaggagtat atgggagtgg ttggagtgag actagagaaa tgtaattaaa 6720
ctataatctc aatacaaaga tctactaagc aaaaaacatg aaacattgtc attcaagtga 6780
aacatcagtc ttcaaattgg aaagatattt ttactaggaa aatgtctggt agatggttat 6840
tatctagaaa acacaaaaat tagaaaacgg taaactttaa taaaaagaat aatacaatga 6900
gactacatga aaagttctta actaatgaaa caaatatctt gaaacttttt tcttaaaagt 6960
ttaatatcaa taaccatcat ggaaattcaa attaaaacta tttacatatt acccctgaaa 7020
taataactaa tacccaataa aaataatata aacaaaaaat ggcaatgcat gccatcatgg 7080
atttgggaga gagaatgttc attgcagttc tgaatggata ctggtgccac cacggtgaaa 7140
atctctgtat aggtccttcc aaaagctgaa aatagacata tcacaagacc tgccacacat 7200
ttttcaagca aatacccaaa ggactctacc tgactgcaga gacactttct cataaaatat 7260
tattgttgat ctattcataa tatctggaaa atagaaacag ccaagatgcc catcaactga 7320
ttaatagatg ataaaattat tgtacatttc agtgtaatat tattcagttt ttaagaaaaa 7380
tgaaattatg taataagcat gtaaatggat atatcttgaa acaaccattc cccattatat 7440
tacctaaaca ttgaaagtcc aaaatcatat gatcttttta gtggatctac taatcttttg 7500
ctatatgtat tttattgaac tacccatgga tgtgagataa ttggtaacaa cagcacatgg 7560
gagagcatgg gatcattcaa ggaagattag agagaatgca ttttttagga gataatggag 7620
gagcaataga aaggattaaa tgaggttact gatgaaagtg atggttagag aaggcaatat 7680
gaggagggat aactagcact tagggccttt tgaaaaagac atagagaaaa tactattgta 7740
gaaacttcct ataattggtg tatagttata tacaccaaag agctcagatg gagttaccct 7800
ataatggaaa tattaactac tttttatcac tgtgataaaa catcctgaac agagcaacat 7860
agattgggaa gcatttactt tggcttacag ttctaacggg ataaaaattc atgatgaaag 7920
aatgaatatg tcagcaaaca gcagtagcaa tggcctgaga agcaggtgag agctcacatc 7980
ttgaagtgta agaatgtagc agagagaaca aactgcaaat gaccagaaaa tgcttttgga 8040
tcagagccca tacccctctg actgacttct ccagaaattc tgaacaaata aaactcccca 8100
aacagagcca taactgaagg tccagtgtct gagactacta ggggtatttc ttattcaaac 8160
cactacaatg gggtgggggg agcaatcctc caagtaggca ctacacacag acaaataaaa 8220
actctagtaa ctggaatgga ttgacttatt tgaattactt gccagtggag ctacatagag 8280
cacaattatt gtatttaaat taccctttat gatcttacaa aacttgacag taagatcata 8340
ttgctaaaga aaccacatat ttgaatcagg gaacatggtg atatctagtt gttcttcaac 8400
tggaaacttc atgctttctg cccagcattc atgttgctgg aaagagcaat gtacactacc 8460
agtgtagaaa ttaaatcatc aatcttatca agatgtggat cctataagtt acaataaaaa 8520
ttagcctgat aagatatccc caccagaaga atattcacat aaatgctatg ggagcaacaa 8580
gctattttct aaattagctt taatcctatt ctacaagaga gaatccatat ctagaatagt 8640
tatagggatc aagaacccat ggcttgattg gtcataggcc caatgggaga tcctaatatt 8700
attgttctac aaaatgaaaa taactcctaa tgacttgttg ctgcagtaat aagttagtat 8760
gttgctcaac tctcacaaga gaagttttgt cttacaataa atggcaatta aagcagcccc 8820
acaagattta tatcataccg atctcctcat ggcctatgca tctagaagct aggaaacaaa 8880
gaggacccta agagagacat acatggtccc cctggagaag gggaaggggg caagacctcc 8940
aaagctaatt gggagcatgg gggaggggag agggagttag aagaaagaga aggggataaa 9000
aggagggaga ggaggacaag agagagaagg aagatctagt caagagaaga tagaggagag 9060
caagaaaaga gataccatag tagagggagc cttgtatgtt taaatagaaa actggcacta 9120
gggaattgtc caaagatcca caaggtccaa ctaataatct aagcaatagt cgagaggcta 9180
ccttaaaagc ctttctctga taatgagatt gatgactacc ttatatacca tcctagagcc 9240
ttcatccagt agctgatgga agcagaagca gacatctaca gctaaacact gagctagttg 9300
cagacaggga ggagtgatga gcaaagtcaa gaccaggctg gagaaacaca cagaaacagc 9360
agacctgaaa aaaatgttgc acatggaccc cagactgata gctgggagtc cagcatagga 9420
cttttctaga aaccctgaat gaggatatca gtttggaggt ctggttaatc tatggggaca 9480
ctggtagtgg atcaatattt atccctagtt catgactgga atttgggtac ccattccaca 9540
tggaggaatt ctctgtcagc ctagacacat gggggaggtt ctaggtcctg ctccaaataa 9600
tgtgttagac tttgaagaac tcccttgaga agactcaccc tccctgggga gcagaaaggg 9660
gatgggatga gggttggtga gggacaggag aggaggggag ggtgagggaa ctgggattga 9720
caagtaaatg atgcttgttt ctaatttaaa tgaataaagg aaaagtaaaa gaagaaaaga 9780
aaacaggcca aaagattata aaagacagag gtggtgggtg actataaaga aacactatta 9840
tctaaataaa aatatgtcag aagcacacat gaacttatag tgtttatgaa agtatgtata 9900
ataactacat aatctcaagc caagaaaaaa atatcatctt tcagtgatga aggtgatttt 9960
atttctccca gaattaaagc caaagaccta atgaaagtaa ttatcttcaa aaggttgaaa 10020
atacatactt tgcaatacac agatctgcct agaaatctca tgttcacaat acacatgatg 10080
ctcaattgaa ttccattcaa tgttacagtt tagataaaca gtttgtagat aaactcacaa 10140
tgtatcattt ctttttattt tttgaccaaa cagcttctca tctgttattc agaataattc 10200
ctcgatggca ggatatccat cccaattggg ggaaggggag aatttgaaga aaacctagac 10260
cacatacata tttgccattg ggaaacaaag tctaaaatga tgttgttcac atcttctcta 10320
ctagtcctct ccccgtccca aagaaccttg gtatatgtgc ctcattttac agagagagga 10380
aagcaggaac tgagcatccc ttacttgcca tcctcaaccc aaaatttgca tcattgctca 10440
gctctgccct tctcatatga cagttacaag tcaaggcttc caaagtccct ctgtcatgtt 10500
tggtgtcaat agtttataca gatgacttca tgtcttcata tctaatgtct tatatagatt 10560
aatattaaac aatgttattt ctctaaccac attttaaatt aatttaaaaa tccattaatt 10620
gtgtctataa aatgcagaca gagtgctgag acacaatata agcctgatga tctgaatttg 10680
aaactcacac ccaccacatg gagaatcaac ttccaaaaat tttcctatta cttccacact 10740
tacaccattg tacaaacaca ataataatga acaaaatgaa atgaaataaa aaattaagtc 10800
tctgtaggta atgctactgt gcagcaaaag taaaaatggc agcttaagct tgctttatgg 10860
ttacacttta ccatcttcca ttaattataa ggacttcaat catggcagaa ctatgctgtt 10920
attgtctcag tgtaacctaa ccaggtgttc cagatgttct taatgtggac acctaaacta 10980
tttgatattt gggttaagat ctttccctct ttcagaagaa acctcaggac agagggaatc 11040
ttgtctttta attttgagtc tgtagacttt ttccatttca aatatacatg aaacaagtga 11100
tgaagaaaat taatcaaaag gtgggaattg caatgatatt aggttcaata ttaagcttca 11160
atattatcat ggaatcgcct gttatacact gagtgtttgg caataaggga tttttagaag 11220
aaggagtttt tattctcaac aggttcctta agtttagctc aaataaatct aagcaatcca 11280
ctctagaatt aaatagtttc ctaagggcac agctatgaat agagctcaat ttacatataa 11340
aattttgttc accatttatg tcattccagt tttcattagt acaaggaaaa tacaaaatat 11400
ttagatgtca atatcaagtg aatagttcat ctcctttttt aatatatatc acctaaatca 11460
ccattttctc agaaaaatct ggcctgaagt tctgtctgga acttcaacat gaaaaatatg 11520
cacagcttgc tattataaat cctagttgat ttttaagatt catgtctggt gtctgactca 11580
gaggggccag aggctagaca aatatttttt gaatcttcat tgtgaagatt tttaatgatt 11640
attttaatat aaataacaaa gatgatggat aatgtaactt tgtacagttc atagacgctg 11700
aactactttg tgcttaaaat gttagttccc tatcataaat gataggtgat aagtgtatgt 11760
ttaatacttt ccctctgagc tatattcatg tactagagaa ttattttaaa catgaaaaga 11820
ctgtgtttat agtctcagct cctgagaact ggtccaacct taggcaggtg aatgccagga 11880
gcaacgtttt tcttctacag aggatgcttt gctgccaagc aacctggttg tgtggaaatg 11940
ttcctttttt aatcaagttt aaagggtctt catcatgctg ttgctccaca tattttcagg 12000
ttagagcttg gtccttggag tattatcttt taccagaaaa ttcatagtat tctttcaata 12060
actaacaact aaacttttcg ataaaaaaga attggaattt caattttaaa gcctgagtaa 12120
aattcttgtg aatcaggata ttttatttta agtcttatct tttaaaaagt tattttattt 12180
tttaaaaaat tataatatac tttcataatt tccctccttc acttttcttt acaaacactt 12240
ctatagatca ccatgtgttt ttttttttac atttatggcc tctttctgtt cattgttatt 12300
acatacaaat agtcttgcct atagaagaac accacaattt gttacctgat aacaaattat 12360
caacccttaa aacctacaaa ctattgatat tactgaaaag actatactta tagatgtaaa 12420
gatatatgtg tgtgcacata tatagataca catatatgta ggatttttaa ttttagattt 12480
tagacatcaa aattatttat atgactgaga aactagacac tataaatgag cattcagtat 12540
tcaacaccgt gattttagat attgtcacaa tgacagaaaa ttttcttata gaaaatttta 12600
agttttgtga ttgctctgtg cacttagtga agtctcacag aaaaagaatc atagtatttt 12660
tagtttataa taaaaagtac atataattaa aatggttggc acaaaacaac atttgagcat 12720
ttttcctatt tactatcaag tagtatcatt ttgaaataat aatttgacta gtttcaaaaa 12780
tgaaaacaaa atttaaacta aatgcctaat ctagcctgat aacattttta tgaatgaaat 12840
tattcaatag tgttatcaat taggggccca aaacttttcc taaaataaaa cttttaattt 12900
ttttccattt ttatttaaat tagaaacaaa attgttttac atgtaaatca gagtttcctc 12960
accctcccct tctccctgtc cctcactaac accctacttg tcccatacca tttctgctcc 13020
ccagggaggg tgaggccttc catggggaaa cttcagagtc tgtctatcct ttcggatagg 13080
gcctaggccc tcacccattt gtctaggcta aggctcacaa agtttactcc tatgctagtg 13140
ataagtactg atctactaca agagacacca tagatttcct aggcttcctc actgacaccc 13200
atgttcatgg ggtctggaac aatcatatgc tagtttccta ggtatcagtc tggggaccat 13260
gagctccccc ttgttcaggt caactgtttc tgtgggtttc accaccctgg tcttgactgc 13320
tttgctcatc actcctccct ttctgtaact gggttccagt acaattccgt gtttagctgt 13380
gggtgtctac ttctactttc atcagcttct gggatggagc ctctaggata gcatacaatt 13440
agtcatcatc tcattatcag ggaagggcat ttaaagtagc ctctccattg ttgcttggat 13500
tgttagttgg tgtcatcttt gtagatctct ggacatttcc ctagtgccag atatctcttt 13560
aaacctacaa gactacctct attatggtat ctcttttctt gctctcgtct attcttccag 13620
acaaaatctt cctgctccct tatattttcc tctcccctcc tcttctcccc ttctcattct 13680
cctagatcca tcttcccttc ccccatgctc ccaagagaga tgttgctcag gagatcttgt 13740
tccttaaccc ttttcttggg gatctgtctc tcttagggtt gtccttgttt cctagcttct 13800
ctggaagtgt ggattgtaag ctggtaatca tttgctccat gtctaaaatc catatatgag 13860
tgatgtttgt ctttttgtga ctgggttacc tcactcaaaa tggtttcttc catatgtctg 13920
tggatttcaa tagcacaaac aacatacagt atcttggggc aacactaacc aaacaagtga 13980
aagaccagta tagcaagaac tttgagttta aagaaagaaa ttaaagaaga taccagaaaa 14040
tggaaagatc tcccatgctc tttgataggc agaatcaaca tagtaaaaat ggcaatcttg 14100
ccaaaatcca tctacagact caatgcaatc cccattaaat accagcacac ttcttcacag 14160
acctgaaaga ataatactta actttatatg gagaaacaaa agacccagga taggccaaac 14220
aaccctgtac aatgaaggca cttccagagg catccccatc cctgacttca agctctatta 14280
tagagtaata atcctgaaaa cagcttggta atggcacaaa aatagacagg tagaccaatg 14340
gaattgagtt gaaaaccctg atattaaccc acatatctat gaacacctga ctttgacaaa 14400
gaagctaagg ttatacaatg taagaaagaa agcatcttca acaaatcgtg ctggcataac 14460
tggatgctgg catgtagaag actgcagata gatccatgtc taatgccatg cacaaaactt 14520
aagtccaaat ggatcaaaaa cctcaacata aatccagcca cactgaacct catagaagag 14580
aaagtgggaa gtatccttga ataaattggt acaggagacc acatcttgaa cttaacacca 14640
gtagcacaga caatcagatc aataatcaat aaatgggacc tcctgaaact gagaagcttc 14700
tgtaaggcaa tggataagtc aacaggacaa aatggcagcc cacggaatgg gaaaagatat 14760
tcaccaatcc tatatctgac agagggctgc tctctatttg caaagaacac aataagctag 14820
tttttaaaac accaattaat ccgattataa agttgggtag agaactaaat aaagaattgt 14880
taacagagca atctaacttg gcagaaagac acataagaaa gtgctcacca t 14931

It is to be understood that the description, specific examples and data, while indicating exemplary embodiments, are given by way of illustration and are not intended to limit the present inventions. Various changes and modifications within the present inventions, including combining embodiments in whole and in part, will become apparent to the skilled artisan from the discussion, disclosure and data contained herein, and thus are considered part of the inventions.

The present inventions may be embodied in other specific forms without departing from the spirit or essential attributes thereof and, accordingly, reference should be made to the appended claims, rather than to the foregoing specification, as indicating the scope of the inventions.

What is claimed is:

1. A method for controlling the transcription of a polynucleotide of interest in a cell, wherein the method comprises
I. maintaining a cell in a medium without an effective amount of a ligand of both an activator and a repressor, wherein the cell comprises:
(A) a promoter operably linked to a polynucleotide of interest and controlled by a first operator operably linked and positioned 5′ with respect to the promoter;
(B) a polynucleotide encoding the activator;
(C) a second operator; and
(D) a polynucleotide encoding the repressor,
wherein transcription of the polynucleotide of interest is inhibited in the absence of the ligand of both the activator and the repressor; and
II. controlling the cell to transcribe the polynucleotide of interest by maintaining the cell in a medium with an effective amount of the ligand of both the activator and the repressor.

2. The method according to claim 1, wherein the second operator is operably linked and positioned 3′ with respect to the promoter and 5′ with respect to the polynucleotide of interest.

3. The method according to claim 1, wherein the activator binds to the first operator in the presence of the ligand to permit transcription of the polynucleotide of interest.

4-9. (canceled)

10. A method for controlling the transcription of a polynucleotide of interest in a cell, wherein the method comprises
I. maintaining a cell in a medium without an effective amount of a ligand of an activator (activator ligand) and with an effective amount of ligand of a repressor (repressor ligand), wherein the cell comprises:
(A) a promoter operably linked to a polynucleotide of interest and controlled by a first operator operably linked and positioned 5′ with respect to the promoter;
(B) a polynucleotide encoding the activator;
(C) a second operator; and
(D) a polynucleotide encoding the repressor,
wherein transcription of the polynucleotide of interest is inhibited in the absence of the activator ligand and the presence of the repressor ligand; and
II. controlling the cell to transcribe the polynucleotide of interest by maintaining the cell in a medium with an effective amount of the activator ligand and without an effective amount of the repressor ligand.

11. The method according to claim 10, wherein the second operator is operably linked and positioned 3′ with respect to the promoter and 5′ with respect to the polynucleotide of interest.

12-62. (canceled)

63. A method for controlling the transcription of a polynucleotide of interest in a cell, wherein the method comprises maintaining a cell in a medium without an effective amount of a first ligand of a first regulatory fusion protein (RFP) and with an effective amount of second ligand of a second RFP, wherein the cell comprises
(A) a promoter;
(B) an Arc operator; and
(C) a polynucleotide encoding a reverse tetracycline transactivator fusion protein (rtTA), wherein (A), (B) and (C) are operably linked, and wherein transcription of the rtTA polynucleotide is controlled by a fusion protein comprising an Arc repressor binding domain and an estrogen receptor ligand binding domain (ArcEr);
wherein rtTA can control the transcription of a polynucleotide of interest.

64. (canceled)

65. The method according to claim 63, wherein the first ligand is selected from the group consisting of tetracycline and doxycycline.

66. The method according to claim 63, wherein the second ligand is selected from the group consisting of estrogen, estradiol (E2), tamoxifen, and 4-hydroxytamoxifen (OHT).

67-107. (canceled)

108. A cell capable of controlled transcription of at least one polynucleotide of interest, wherein the cell comprises:
(A) a promoter operably linked to a polynucleotide of interest and controlled by a first operator operably linked and positioned 5′ with respect to the promoter;
(B) a polynucleotide encoding an activator;
(C) a second operator; and
(D) a polynucleotide encoding a repressor,
wherein transcription of the polynucleotide of interest is inhibited in the absence of a ligand of both the activator and the repressor, and is permitted in the presence of the ligand of both the activator and the repressor.

109-111. (canceled)

112. A cell capable of controlled transcription of a polynucleotide of interest, wherein the cell comprises
(A) a promoter operably linked to a polynucleotide of interest and controlled by a first operator operably linked and positioned 5′ with respect to the promoter;
(B) a polynucleotide encoding an activator;
(C) a second operator; and
(D) a polynucleotide encoding a repressor;
wherein transcription of the polynucleotide of interest is inhibited in the absence of an effective amount if an activator ligand and the presence of an effective amount of a repressor ligand; and permitted in the presence of an effective amount of the activator ligand and the absence of an effective amount of the repressor ligand.

113. The cell according to claim 112, wherein the second operator is operably linked and positioned 3′ with respect to the promoter and 5′ with respect to the polynucleotide of interest.

114. The cell according to claim 112, wherein the activator binds to the first operator in the presence of the activator ligand to permit transcription of the polynucleotide of interest.

115. (canceled)

116. A cell capable of controlled transcription of a polynucleotide of interest, wherein the cell comprises:
(A) a promoter operably linked to a polynucleotide of interest and controlled by a first operator operably linked and positioned 5′ with respect to the promoter;
(B) a polynucleotide encoding a first regulatory fusion protein (RFP), where the first RFP comprises:
(1) a transcription activating domain fused to a first DNA binding domain; and
(2) a ligand-binding domain; wherein the first ligand is capable of binding to the ligand-binding domain of the first RFP, and wherein the DNA binding domain of the first RFP is capable of binding to the operator positioned 5′ when in the presence of the first ligand;
(C) a second operator; and
(D) a polynucleotide encoding the second RFP that differs from the first RFP, wherein the second RFP comprises:
(1) a DNA-binding domain; and
(2) a ligand-binding domain; wherein the second ligand is capable of binding to the ligand-binding domain of the second RFP, and wherein the second RFP is capable of binding to the second operator in the presence of the second ligand; wherein transcription of the polynucleotide is inhibited in the absence of an effective amount of the first ligand and in the presence of an effective amount of the second ligand and is permitted in the presence of an effective amount of the first ligand and absence of an effective amount of the second ligand.

117. The cell according to claim 116, wherein the second operator is operably linked and positioned 3′ with respect to the promoter and 5′ with respect to the polynucleotide sequence encoding the protein of interest.

118. The cell according to claim 116, wherein the second operator is operably linked to the polynucleotide sequence encoding the first RFP, and the second RFP can control transcription of the polynucleotide encoding the first RFP.

119. (canceled)

120. A cell capable of controlled transcription of a polynucleotide of interest, wherein the cell comprises
(A) a promoter operably linked to a polynucleotide of interest and controlled by a first operator operably linked and positioned 5′ with respect to the promoter;
(B) a polynucleotide encoding a regulatory fusion protein (RFP), wherein the RFP comprises:
(1) a transcription activating domain fused to a DNA binding domain; and
(2) a ligand-binding domain; wherein the ligand is capable of binding to the ligand-binding domain of the RFP, and wherein the DNA binding domain of the RFP is capable of binding to the first operator when in the presence of the ligand; and
(C) a second operator; and
(D) a polynucleotide encoding a repressor protein, wherein the repressor protein can bind to the second operator only in the absence of the ligand,
wherein transcription of the polynucleotide of interest is inhibited in the absence of an effective amount of the ligand of both the activator and the repressor, and is permitted in the presence if an effective amount of the ligand of both the activator and the repressor.

121. The cell according to claim 120, wherein the second operator is operably linked and positioned 3′ with respect to the promoter and 5′ with respect to the polynucleotide of interest.

122-136. (canceled)

137. A cell capable of controlled transcription of a polynucleotide of interest, wherein the cell comprises
(A) a promoter;
(B) an Arc operator; and
(C) a polynucleotide encoding a reverse tetracycline transactivator fusion protein (rtTA), wherein (A), (B) and (C) are operably linked, wherein transcription of the rtTA polynucleotide is controlled by a fusion protein comprising an Arc repressor binding domain and an estrogen receptor ligand binding domain (ArcEr), and
wherein rtTA can control the transcription of a polynucleotide of interest.

138-139. (canceled)

140. A bioreactor comprising (i) a cell according to claim 108 and (ii) media.

141-144. (canceled)

145. A bioreactor comprising (i) a cell according to claim 112 and (ii) media.

146. A bioreactor comprising (i) a cell according to claim 116 and (ii) media.

147. A bioreactor comprising (i) a cell according to claim 120 and (ii) media.

148. A bioreactor comprising (i) a cell according to claim 137 and (ii) media.


SEQUENCE LISTING (18 sequences)

SEQ ID NO: 1 - DNA, 1008 bp, synthetic construct
  misc_feature 1..1008: note = Synthetic: rtTA nucleotide sequence
  source 1..1008: mol_type = other DNA
atgtctagactggataagtctaaggtgatcaatggagctctggaactgctgaatggagtgggaatcgaaggactgacaacaagaaagctggctcagaagctgggagtggaacagcctacactgtattggcatgtgaagaataagagagctctgctggatgctctgcctatcgaaatgctggatagacatcatacacatttttgtcctctggaaggagaatcttggcaggattttctgagaaataatgctaagtcttttagatgtgctctgctgtctcatagagatggagctaaggtgcatctgggaacaagacctacagaaaagcagtatgaaacactggaaaatcagctggcttttctgtgtcagcagggattttctctggaaaatgctctgtatgctctgtctgctgtgggacattttacactgggatgtgtgctggaagaacaggaacatcaggtggctaaggaagaaagagaaacacctacaacagattctatgcctcctctgctgagacaggctatcgaactgtttgatagacagggagctgaacctgcttttctgtttggactggaactgatcatctgtggactggaaaagcagctgaagtgtgaatctggatctgcttattctagagctagaacaaagaataattatggatctacaatcgaaggactgctggatctgcctgatgatgatgctcctgaagaagctggactggctgctcctagactgtcttttctgcctgctggacatacaagaagactgtctacagctcctcctacagatgtgtctctgggagatgaactgcatctggatggagaagatgtggctatggctcatgctgatgctctggatgattttgatctggatatgctgggagatggagattctcctggacctggatttacacctcatgattctgctccttatggagctctggatatggctgattttgaatttgaacagatgtttacagatgctctgggaatcgatgaatatggaggataa

SEQ ID NO: 2 - protein, 335 aa, synthetic construct
  REGION 1..335: note = Synthetic: rtTA amino acid sequence
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 3 - DNA, 1341 bp, synthetic construct
  misc_feature 1..1341: note = Synthetic: ArcER nucleotide sequence
  source 1..1341: mol_type = other DNA
atgaagggcatgtccaagatgcctcagttcaacctgcgctggcctcgcgaggtgctggacctggtgcgcaaggtggccgaggagaacggccgctccgtgaactccgaaatctaccagcgcgtgatggagtccttcaagaaggagggccgcatcggagccggaggtggctccggaggtggcaccggtggaggctctggaggaggcatgaaaggaatgtctaaaatgccccaatttaatctccggtggccccgcgaagtcctcgatctcgtgcggaaagtcgctgaagaaaatggacggtctgtcaattctgaaatttatcaacgggtcatggaatcttttaaaaaagaaggacggattggagctgcttattctggatcccgggaattaattcggctttctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 4 - protein, 446 aa, synthetic construct [sequence omitted]

SEQ ID NO: 5 - DNA, 657 bp, synthetic construct
  misc_feature 1..657: note = Synthetic: TetR Nucleotide Sequence
  source 1..657: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaa

SEQ ID NO: 6 - protein, 218 aa, synthetic construct
  REGION 1..218: note = Synthetic: TetR Amino Acid Sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSGSREFRSY

SEQ ID NO: 7 - DNA, 19 bp, synthetic construct
  misc_feature 1..19: note = Synthetic: Tet Operator
  source 1..19: mol_type = other DNA
tccctatcagtgatagaga

SEQ ID NO: 8 - DNA, 235 bp, synthetic construct
  misc_feature 1..235: note = Synthetic: Tet Response Element
  source 1..235: mol_type = other DNA
tccctatcagtgatagagaacgtatgaagagtttactccctatcagtgatagagaacgtatgcagactttactccctatcagtgatagggaacgtataaggagtttactccctatcagtgatagagaacgtatgaccagtttactccctatcagtgatagagaacgtatctacagtttactccctatcagtgatagagaacgtatatccagtttactccctatcagtgatagaga

SEQ ID NO: 9 - DNA, 23 bp, synthetic construct
  misc_feature 1..23: note = Synthetic: Arc Operator
  source 1..23: mol_type = other DNA
atgatagaagcactctactattc

SEQ ID NO: 10 - DNA, 50 bp, synthetic construct
  misc_feature 1..50: note = Synthetic: hCMVmin promoter
  source 1..50: mol_type = other DNA
gcgtataagctttaggcgtgtacggtgggaggcctatataagcagagctc

SEQ ID NO: 11 - DNA, 6473 bp, Cricetulus griseus
  source 1..6473: mol_type = genomic DNA
tctagaaacaaaaccaaaaatattaagtcaggcttggcttcaggtgctggggtggagtgctgacaaaaatacacaaattcctggctttctaaggctttttcggggattcaggtattgggtgatggtagaataaaaatctgaaacataggtgatgtatctgccatactgcatgggtgtgtatgtgtgtgtatgtgtgtctgtgtgtgtgcccagacagaaataccatgaaggaaaaaaacacttcaaagacaggagagaagagtgacctgggaaggactccccaatgagatgagaactgagcacatgccagaggaggtgaggactgaaccattcaacacaagtggtgaatagtcctgcagacacagagagggccagaagcactcagaactccagggggtcaggagtggttctctggaggcttctgcccttggaggttcctgaggaggaggcttccatattgaaaatgtagttagtggccgtttccattagtacagtgactagagagagctgagggaccactggactgaggcctagatgctcagtcagatggccatgaaagcctagacaagcacttccgggtggaaaggaaacagcaggtgtgaggggtcaggggcaagttagtgggagaggtcttccagatgaagtagcaggaacggagacgcactggatggccccacttgtcaaccagcaaaagcttggatcttgttctaagaggccagggacatgacaagggtgatctcggtttttaaaaggctttgtgttacctaatcacttctattagtcagatactttgtaacacaaatgagtacttggcctgtattttagaaacttctgggatcctgaaaaaacacaatgacattctggctgcaacacctggagactcccagccaggccctggacccgggtccattcatgcaaatactcagggacagattcttcactaggtactgatgagctgtcttggatgcaaatgtggcctcttcattttactacaagtcaccatgagtcaggaggtgctgtttgcacagtgtgactaagtgatggagtgttgactgcagccattcccggccccagcttgtgagagagatccttttaaattgaaagtaagctcaaagttaccacgaagccacacatgtataaactgtgtgaataatctgtgcacatacacaaaccatgtgaataatctgtgtacatgtataaactgtgtgaataatctgtgtgcagcctttccttacctactaccttccagtgatcaggtttggactgcctgtgtgctactggaccctgaatgtccccaccgctgtcccctgtcttttacgattctgacatttttaataaattcagcggcttcccctctgctctgtgcctagctataccttggtactctgcattttggtttctgtgacatttctctgtgactctgctacattctcagatgacatgtgacacagaaggtgttccctctggagacatgtgatgtccctgtcattagtggaatcagatgcccccaaactgttgtccagtgtttgggaaagtgacacgtgaaggaggatcaggaaaagaggggtggaaatcaagatgtgtctgagtatctcatgtccctgagtggtccaggctgctgacttcactcccccaagtgagggaggccatggtgagtacacacacctcacacatactatatccaacacacacacacacacacacacacacacgcacgcacgcacgcacgcacgcacacatgcacacacacgaactacatttcacaaaccacatacgcatattacaccccaaacgtatcacctatacataccacacatacacacccctccacacatcacacacataccacacccacacacagcacacacatacataggcacacattcacacaccacacatatacatttgtgtatgcatacatgcatacacacacaggcacacagacaccacacacatgcattgtgtacgcacacatgcatacacacacataggcacacattgagcacacacatacatttgtgtacgcacactacatagacatatatgcatttgtatatgcacacatgcatgcacacatacataggcacacatagagcacacacatacatttgtgtatgcacacatgcacacaccaatcacatgggaagactcaggttcttcactaaggttcacatgaacttagcagttcctggttatctcgtgaaacttggaagattgctgtggagaagaggaagcgttggcttgagccctggcagcaattaaccccgcccagaagaagtaggtttaaaaatgagagggtctcaatgtggaacccgcagggcgccagttcagagaagagacctacccaagccaactgagagcaaaggcagagggatgaacctgggatgtagtttgaacctctgtaccagctgggcttcatgctattttgttatatctttattaaatattcttttagttttatgtgcgtgaataccttgcttgcataaatgtatgggcactgtatgtgttcttggtgccggtggaggccaggagagggcatggatcctccggagctggcgtttgagacagttgtgacccacagtgtggggtctgggaactgggtcttagtgttccgcaagtgcagctggggctcttaacctctgagccatccctccagcttcaagaaacttattttcttaggacatgggggaagggatccagggctttaggcttgtttgttcagcaaatactcttttcgtgtattttgaattttattttattttacttttttgggatagaatcacattctgcagctcaggctgggcctgaactcatcaaaatcctcctgtctcagtctaccaggtgataagattactgatgtgagcctggctttgacaagcactttagagtccccagcccttctggacacttgttccaagtataatatatatatatatatatatatatatatatatatatatatatattgtgtgtgtgtgtttgtgtgtgtatgagacacttgctctaagggtatcatatatatccttgatttgcttttaatttattttttaattaaaaatgattagctacatgtcacctgtatgcgtctgtatcatctatatatccttccttccttctctctctttctctcttcttcttctcacccccaagcatctattttcaaatccttgtgccgaggagatgccaagagtctcgttgggggagatggtgagggggcgatacaggggaagagcaggaggaaagggggacagactggtgtgggtctttggagagctcaggagaatagcagcgatcttccctgtccctggtgtcacctcttacagccaacaccattttgtggcctggcagaagagttgtcaagctggtcgcaggtctgccacacaaccccaatctggccccaagaaaaggcacctgtgtgtgactctggggttaaaggcgctgcctggtcgtctccagctggacttgaaactcccgtttaataaagagttctgcaaaataatacccgcagagtcacagtgccaggttcccgtgctttcctgaagcgccaggcacgggttccctaggaaatggggccttgcttgccaagctcccacggcttgccctgcaaacggcctgaatgatctggcactctgcgttgccactgggatgaaatggaaaaaagaaaaagaagaagtgtctctggaagcgggcgcgctcacacaaacccgcaacgattgtgtaaacactctccattgagaatctggagtgcggttgccctctactggggagctgaagacagctagtgggggcggggggaggaccgtgctagcatccttccacggtgctcgctggctgtggtgcatgccgggaaccgaaacgcggaactaaagtcaagtcttgctttggtggaactgacaatcaacgaaatcacttcgattgttttcctctttttactggaattcttggatttgatagatgggggaggatcagagggggaggggaggggcggggagacggagggaggaggggaggaggggaggaggggaggaggggaggaggggaagggatggaggaaaatactaacttttctaattcaacatgacaaagattcggagaaagtgcaccgctagtgaccgggaggaggaatgccctattgggcattatattccctgtcgtctaatggaatcaaactcttggttccagcaccaaggattctgagcctatcctattcaagacagtaactacagcccacacggaagaggctatacaactgaagaaataaaattttcactttatttcatttctgtgactgcatgttcacatgtagagagccacctgtgtctaggggctgatgtgctgggcagtagagttctgagcccgttaactggaacaacccagaactcccaccacagttagagcttgctgagagagggaggcccttggtgagatttctttgtgtatttatttagagacagggtctcatactgtagtccaagctagcctccagctcacagaaattctcctgttccggtttccaaagtactggagttatgagtgtgtgttaattgaacgctaagaatttgctgattgaagaaaacctcaagtgggtttggctaatccccacgaccccagaggctgaggcaggaggaatgagagaattcaaggtttgccagagccacagggtgagctcaatgtggagactgtgagggtgagctcaatgtggagactgtgagggtgagctcaatgtggagactgtgagggtgagctcaatgtggagactgtgagggtgagctcaatgtggagactgtgagggtgagctcaatgtggagacctgtatcaagataataatagtagtagtaacaatgcaggcgagggtgtggttgagtggtagagcagttagttgatttgacatgcttgaggtctcccggtccatctgtggccctgcaacaggaagggagggaggaagggggggaacgagagagaggaaagagagacagaagctaagatagggaatgagagaggaaggaagaaacgggaagaaattcagactccttcctgagttccgccaacgcctagtgacatcctgtgcacaccctaaggtggcctttgtgtggcactggcttgggtggtcgggaaaggcattttcagcttgttgcagaactgccacagtagcatgctgggtccgtgaaagtttctgcccgttaacaagaagtctctactacttgtgacctcaccagtgaaaatttctttaattgtctcctggtgttctgggttttgcatttttgtttctaaggatacattcctgggtgatgtcatgaagtccccaaagacacagtggggctgtgttggattgggaaagatgatttatctggggtgtcaaaaggaaaagaagggaaacaggcacttgggaaaatgtcctcccgcccacccgaattttggcttggcaaccgtggtggaggagcaagaaacacgtggacgtttgaggaggcatggggtcctaggaggacaggaagcagaaggagagagctgggctgacagcctgcaggcattgcacagtttcagaaggagattacagcatgactgagtttttagggatccaacagggacctgggtagagattctgtgggctctgaggcaacttgacctcagccagatggtatttgaataacctgctcttagagggaaaacagacatagcaaacagagccacgtttagtgatgaaactctcactttgcctgagtcatgtgcggccatgcccaggggtcaggctgacactcaactcaaaaacaagtgagaaattgaagacaatccgtggtggcagctactggaagggccaccacatccccagaaagagtggagctgctaaaaagccatttgtgataggcacagttatcttgaatgcatggagcagagattacggaaaaatcgagaatgttaatgaggcaacattcgagttgagtcattcagtgtgggaaacccagacgcttccatcccctaaaaggaacatcttgctctcagtcaaaatggaaataaaaattggggcttgaatttggcaaatgattcagaactctgtgtaggtattttcacacgcacagtggataattttcatgttggagtttatttgtgctaaaaggcagaaaagggtaaaaagcacatcttaagagttatgaggttctacgaataaaaataatgttacttacagctattccttaattagtacccccttccacctgtggtaatttcctgagatagtcagtggggaaaagatctctccttctcttctttctccccctcccctcctctccctccctccctccctccctccctcctctccctccctccccctttccttctttctttgctccttctcctctgcctccttctccctttcttcttcatttattctaagtagcttttaacagcacaccaattacctgtgtataacgggaaaacacaggctcaagcagcttagagaagattgatctgtgttcact

SEQ ID NO: 12 - DNA, 7045 bp, Cricetulus griseus
  source 1..7045: mol_type = genomic DNA
actagcgtgcaattcagaggtgggtgaagataaaaggcaaacatttgaggccatttccttatttggcacggcacttaggaagtggaacatgcctaatctactggtttgtaccacctttccctataatggactgtttgggaagctcctgggcaaccgattctggcatctcattggtcagaggcctgttaaatggtactcttatttgcaaagaaggctgtaacttgtagctttaaaagcctctcctcaagaaagaagggagaaaggatatggctagacatatctaatagacttaaccactgtgaaaagccttagtatgaatcagatagaacctatttttaactcagttttgaaaaaaataatctttatatttatttgtgtgtgtgtgtgtgtgtgtgtgtgtgtgtgtgtgtgtgtgtgtgtgaaccacatgtagcaggtgctggaggaggccagaagagggcaccagatctcctggaactgacaccacacatggttatgagctgcctgatgtgggtgctgggaactgaactctcgtgttctgcaagagcagcaactgttctcttaactgatgagccatctctccagccccccccataattttaattgttcattttagtaaattttattcataatcaattatcacagtataaaacaatgattttatatatatcatatacatatcaaggatgacagtgagggggatatgtgtgtgtgtgtgtgtgtgtgtgtgtgtgtgtgtgtgtgttatttgtgtgtgtgctttttaagaaggtgccatagtcactgcatttctctgaaggatttcaaaggaatgagacatgtctgtctgccaggaaccctatcttcctctttgggaatctgacccaaatgaggtattctgaggaactgaatgaagagctcaagtagcagtgtcttaaacccaaatgtgctgtctagagaaagtcaacgtcatcagtgagctgaggagagatttactgagcggaagacaagcgctctttgatttaagtggctcgaacagtcacggctgtggagtggagcctgtgctcaggtctgaggcagtctttgctagccagctgtgatgagcagtgaagaaagggtggagatggaggcagggtgggagcagggctatggttcagactaggtatcgtgagcacaccagctggttgacttgtggtctgtgggtcaggcgttgtaaacgccctcagggtcaggcagtcacattgcttgaagctgaatgggtgaggcaacacagagagtgcaaagaaggcaaagtaccacctcttccccgacccaggtcacttctgggttatagctgagactccggacagcatgcaaccagctggttagagcttcagggaaaacttgatgtctgcatgttgctatgaaatgtgattcggtacatctggagaaaatttataatgctggctcagtcaagcactgaacaaaggtaccttggctttgggagctacatgacattgacttgtaggcagacttttttttttctgcccgccaattcccagataaccaatatggaggctcaatattaattataaatgctcggctgatagctcaggcttgttactagctaactcttccaacttaaatgaacccatttctattatctacattctgccacgtgactttaccttgtacttcctgtttcctctccttgtctgactctgcccttctgcttcccagagtccttagtctggttctcctgcctaaccttatcctgcccagctgctgaccaagcatttataattaatattaagtctcccagtgagactctcatccagggaggacttgggtgctcccccctcctcattgccatccgtgtcttcctcttccctcgcttccccctcctcttcctgctcttcctcctccacccctcctttcatagtattgatggcaagggtgttctagaatggaggagtgcccataggcatgcaaagaaaccagttaggatgctctgtgaggggttgtaatcataagcgatggacacaattcaagccacagagtgaagacggaaggatgcactgtgctctagagcaacttctggggcagaatcacagggtgagtttctgacttgagggcgaagaggccacgaggaagggagtgagtttgtctgagctagaagctacggcccacctcttggtagcagacctgcccacaagcatgctttgttaatcatgtgggatctgattttcctctaaatctatgttcaactcttaagaaaatgtgaattctcacattaaaatttagatatacgtcttttggtggggggggtgtaaaaaatcctcaagaatatggatttctgggggccggagagatggctcagaggttaagagaactggttgctcttctagacattctgagttcaattcccagcaaccacatggtggctcacaaccatctgtaatgcgacctggtgccatcttctgacatgcatggatacatgcaggcagaaagctgtatacatagtaaattgataaatctttttttaaaaagagtatggattctgccgggtgttggtggcgcacgcctttaatcccagcactctggaggcagaggcaggtggatctctgtgagttcgagaccagcctggtctataagagctagttccaggacagcctccaaagccacagagaaaccctgtctcgaaaaaccaaaaaaaaaaaaaaaaaaaaaaaaaaaaaaagagtatggattctaagaaagccgtaacagctggagctgtgtacggagttcagcgtggtactagaagaacagacattcatgatgaaacaccccaggatttttacttagtatctagtttccattgttgttttgagaccggctcttatgctctccaggctggcctcaaactgctgatcttcccgcctctacctctcaagtcctgggactacttggctcataaaacagtttttgtcgggctccctgaagttatggttgtacaaaccgtgggggtcaatatactcacttgggcagagagagaaggtctgaatcccagacaatgactgcatctcaggacagttgggaagaggacaatggcagaaggacttagaaaagatagactggagggtggaaaagcagcaggaacagagaaacaaaacaggaagcttgctatccagggccactctggagtcctgtggcaagatggaagcgggctaggggaatacatttgtgctactgtgtgtgtgtgtgtgtgtgtgtgtgtgtgtgtgtgtgatcaatgcctatcaatgttgaaggggaaatatgtataccacattgattctgggagcaattctcagtatctggcctagagaaaggaatggcccctgcagaatagacagagtgaatggtgccctttatcatttgctaaagtgaaggagaaataaacatccttccatagagtttcaggtaaatgaaccccacagttcatctgtgccgtggtggaggcctggccaacagttaaaaagattagacacggacaaagtctgaaggaaacacctcgaataggaagaggagagccacctcattctgtaactttcctcaaggggaagatgttccaagagtgggaataaatggtcaaaggggggatttttaattaggaaaacgatttcctgtatcacttgtgaaactggaggttgatttggggcataggacaatagatttgatgctttgcaaaaagctgtttcaaagcagagaaatggaatagagacaattatgtagcgaggagggagggtggggcgaagatggagacagagaagtggaagctgactttagggaagaggaacatagaccacaggggcggggcggggggcaggggcggggggcggggctcaaaggaggcagtgggaacgttgctagtgttcgcagcgtaagcgtgaatgtgcaagcgtctttgtggtgtgtgaccaggagtagcgtggctggcttgtgtgctgcttgtaatcccagtctttgaggtttccacactgttccacagtgggtgtgattttccctcggagagcatgagggctctgctttccccacatcctccccagcgttcgttggtatttgtttccaagatgttagtgggtgagacaaagcctctctgttgatttgcctttaacaggtgacaaaaaaagctcaaccaggagacatttttgccttcttggaaggtaatgctcccatgtagagcaatgggacccatctctaaggtgaggctactcttgcagtttgcacccagctcttctgatgcaggaaggaagttggtgggcaagcaagactgtttgcttcttgcgatggacacattctgcacacaaaggctcaggaggggagaaggctgtttgatgtttagcactcaggaaggcccctgatgcatctgtgattagctgtctccatctgtggagcagacacggactaactaaaaaccagtgtttttaaattgtcaagcctttaaggtgaggaaattgacttattgtgctgggccatacgtagagcaagtgctctgcattgggccaacccccggctctggtttctaggcaccagaatggcctagaactaactcacaatcctcccattccaggtctcaggtgctagaatgaaccactataccagcctgcctgcctgcctacctgccttcctaaattttaaatcatggggagtaggggagaatacacttatcttagttagggtttctattgctgtgaagagacaccatgagcatggcaactcttataaaggaaaacatttagttgggtggcagtttcagaggttttagtacattgtcatcatggctgggaacatgatggcatgcagacagacatggtgctggagaaagggatgagagtcctacatcttgcaggcaacaggacctcagctgagacactggctggtaccctgagcataggaaacctcacagcccaccctcacagtgacatatttccttcaacaaagccatacctcctaatagtgccactccctatgagatgacagggccaattacattcaaactgctataacactttaaagtattttatttttattattgtaaattatgtatgtagctgggtggtggcagccgaggtgcacgcctttaatcccagcacttgggaggcagaggcagatggatctctgtgagttcaagaccagcctggtctataagagctagttgcaaggaaggatatacaaagaacagttctaggatagccttcaaagccacagagaagtgctgtcttgaaaaccaaaaattgtgctgggacctgtctctgctttggttgcttcccactcccccagagctggactcttggtcaacactgaatcagctgcaaaataaactcctggattcctctcttgtaacaggagcccgaagtcaggcgcccacttgtcttctcgcaggattgccatagactttttctgtgtgcccaccattccagactgaagtagagatggcagtggcagagactgggaaggctgcaacgaaaacaggaagttattgcaccctgggaatagtctggaaatgaagcttcaaaacttgcttcatgttcagttgtacacagactcactcccaggttgactcacacgtgtaaatattcctgactatgtctgcactgcttttatctgatgcttccttcccaaaatgccaagtgtacaaggtgagggaatcacccttggattcagagcccagggtcgtcctccttaacctggacttgtctttctccggcagcctctgacacccctccccccattttctctatcagaaggtctgagcagagttggggcacgctcatgtcctgatacactccttgtcttcctgaagatctaacttctgacccagaaagatggctaaggtggtgaagtgtttgacatgaagacttggtcttaagaactggagcaggggaaaaaagtcggatgtggcagcatgtacccgaaatcccagaactggggaggtagagacggatgagtgcccggggctagctggctgctcagccagcctagctgaattgccaaattccaactcctattgaaaaacctttaccaaacaaacaaacaaacaaataataacaacaacaacaacaacaaactaccccatacaaggtgggcggctcttggctcttgaggaatgactcacccaaacccaaagcttgccacagctgttctctggcctaaatggggtgggggtggggcagagacagagacagagagagacatgacttcctgggctgggctgtgtgctctaggccaccaggaactttcctgtcttgctctctgtctggcacagccagagcaccagcacccagcaggtgcacacacctccctccgtgcttcttgagcaaacacaggtgccttggtctgtctattgaaccggagtaagttcttgcagatgtatgcatggaaacaacattgtcctggttttatttctactgttgtgataaaaaccggggaactccaggaagcagctgaggcagaggcaaatgcaaggaatgctgcctcctagcttgctccccatggcttgccgggcctgctttctgcaagcccttctctccccattggcatgcctgacatgaacagcgtttgaaatgctctcaaatgtcactttcaaagaaggcttctctgatcttgctaactaaatcagaccatgtttcaccgtgcattatctttctgctgtctgtctgtctgtctgtctgtctatctgtctatcatctatcaatcatctatctatctatcttctatttatctacctatcattcaatcatctatcttctaactagttatcatttatttatttgtttacttactttttttatttgagacagtatttctctgagtgacagccttggctgtcctggaacccattctgtaaccaggctgtcctcaaactcacagagatccaactgcctctgcctctctggtgctggggttaaagacgtgcaccaccaacgccccgctctatcatctatttatgtacttattattcagtcattatctatcctctaactatccatcatctgtctatccatcatctatctatctatctatctatctatctatctatctatcatccatctataatcaattg

SEQ ID NO: 13 - DNA, 6473 bp, Cricetulus griseus
  source 1..6473: mol_type = genomic DNA
agtgaacacagatcaatcttctctaagctgcttgagcctgtgttttcccgttatacacaggtaattggtgtgctgttaaaagctacttagaataaatgaagaagaaagggagaaggaggcagaggagaaggagcaaagaaagaaggaaagggggagggagggagaggagggagggagggagggagggagggagaggaggggagggggagaaagaagagaaggagagatcttttccccactgactatctcaggaaattaccacaggtggaagggggtactaattaaggaatagctgtaagtaacattatttttattcgtagaacctcataactcttaagatgtgctttttacccttttctgccttttagcacaaataaactccaacatgaaaattatccactgtgcgtgtgaaaatacctacacagagttctgaatcatttgccaaattcaagccccaatttttatttccattttgactgagagcaagatgttccttttaggggatggaagcgtctgggtttcccacactgaatgactcaactcgaatgttgcctcattaacattctcgatttttccgtaatctctgctccatgcattcaagataactgtgcctatcacaaatggctttttagcagctccactctttctggggatgtggtggcccttccagtagctgccaccacggattgtcttcaatttctcacttgtttttgagttgagtgtcagcctgacccctgggcatggccgcacatgactcaggcaaagtgagagtttcatcactaaacgtggctctgtttgctatgtctgttttccctctaagagcaggttattcaaataccatctggctgaggtcaagttgcctcagagcccacagaatctctacccaggtccctgttggatccctaaaaactcagtcatgctgtaatctccttctgaaactgtgcaatgcctgcaggctgtcagcccagctctctccttctgcttcctgtcctcctaggaccccatgcctcctcaaacgtccacgtgtttcttgctcctccaccacggttgccaagccaaaattcgggtgggcgggaggacattttcccaagtgcctgtttcccttcttttccttttgacaccccagataaatcatctttcccaatccaacacagccccactgtgtctttggggacttcatgacatcacccaggaatgtatccttagaaacaaaaatgcaaaacccagaacaccaggagacaattaaagaaattttcactggtgaggtcacaagtagtagagacttcttgttaacgggcagaaactttcacggacccagcatgctactgtggcagttctgcaacaagctgaaaatgcctttcccgaccacccaagccagtgccacacaaaggccaccttagggtgtgcacaggatgtcactaggcgttggcggaactcaggaaggagtctgaatttcttcccgtttcttccttcctctctcattccctatcttagcttctgtctctctttcctctctctcgttcccccccttcctccctcccttcctgttgcagggccacagatggaccgggagacctcaagcatgtcaaatcaactaactgctctaccactcaaccacaccctcgcctgcattgttactactactattattatcttgatacaggtctccacattgagctcaccctcacagtctccacattgagctcaccctcacagtctccacattgagctcaccctcacagtctccacattgagctcaccctcacagtctccacattgagctcaccctcacagtctccacattgagctcaccctgtggctctggcaaaccttgaattctctcattcctcctgcctcagcctctggggtcgtggggattagccaaacccacttgaggttttcttcaatcagcaaattcttagcgttcaattaacacacactcataactccagtactttggaaaccggaacaggagaatttctgtgagctggaggctagcttggactacagtatgagaccctgtctctaaataaatacacaaagaaatctcaccaagggcctccctctctcagcaagctctaactgtggtgggagttctgggttgttccagttaacgggctcagaactctactgcccagcacatcagcccctagacacaggtggctctctacatgtgaacatgcagtcacagaaatgaaataaagtgaaaattttatttcttcagttgtatagcctcttccgtgtgggctgtagttactgtcttgaataggataggctcagaatccttggtgctggaaccaagagtttgattccattagacgacagggaatataatgcccaatagggcattcctcctcccggtcactagcggtgcactttctccgaatctttgtcatgttgaattagaaaagttagtattttcctccatcccttcccctcctcccctcctcccctcctcccctcctcccctcctccctccgtctccccgcccctcccctccccctctgatcctcccccatctatcaaatccaagaattccagtaaaaagaggaaaacaatcgaagtgatttcgttgattgtcagttccaccaaagcaagacttgactttagttccgcgtttcggttcccggcatgcaccacagccagcgagcaccgtggaaggatgctagcacggtcctccccccgcccccactagctgtcttcagctccccagtagagggcaaccgcactccagattctcaatggagagtgtttacacaatcgttgcgggtttgtgtgagcgcgcccgcttccagagacacttcttctttttcttttttccatttcatcccagtggcaacgcagagtgccagatcattcaggccgtttgcagggcaagccgtgggagcttggcaagcaaggccccatttcctagggaacccgtgcctggcgcttcaggaaagcacgggaacctggcactgtgactctgcgggtattattttgcagaactctttattaaacgggagtttcaagtccagctggagacgaccaggcagcgcctttaaccccagagtcacacacaggtgccttttcttggggccagattggggttgtgtggcagacctgcgaccagcttgacaactcttctgccaggccacaaaatggtgttggctgtaagaggtgacaccagggacagggaagatcgctgctattctcctgagctctccaaagacccacaccagtctgtccccctttcctcctgctcttcccctgtatcgccccctcaccatctcccccaacgagactcttggcatctcctcggcacaaggatttgaaaatagatgcttgggggtgagaagaagaagagagaaagagagagaaggaaggaaggatatatagatgatacagacgcatacaggtgacatgtagctaatcatttttaattaaaaaataaattaaaagcaaatcaaggatatatatgatacccttagagcaagtgtctcatacacacacaaacacacacacacaatatatatatatatatatatatatatatatatatatatatatattatacttggaacaagtgtccagaagggctggggactctaaagtgcttgtcaaagccaggctcacatcagtaatcttatcacctggtagactgagacaggaggattttgatgagttcaggcccagcctgagctgcagaatgtgattctatcccaaaaaagtaaaataaaataaaattcaaaatacacgaaaagagtatttgctgaacaaacaagcctaaagccctggatcccttcccccatgtcctaagaaaataagtttcttgaagctggagggatggctcagaggttaagagccccagctgcacttgcggaacactaagacccagttcccagaccccacactgtgggtcacaactgtctcaaacgccagctccggaggatccatgccctctcctggcctccaccggcaccaagaacacatacagtgcccatacatttatgcaagcaaggtattcacgcacataaaactaaaagaatatttaataaagatataacaaaatagcatgaagcccagctggtacagaggttcaaactacatcccaggttcatccctctgcctttgctctcagttggcttgggtaggtctcttctctgaactggcgccctgcgggttccacattgagaccctctcatttttaaacctacttcttctgggcggggttaattgctgccagggctcaagccaacgcttcctcttctccacagcaatcttccaagtttcacgagataaccaggaactgctaagttcatgtgaaccttagtgaagaacctgagtcttcccatgtgattggtgtgtgcatgtgtgcatacacaaatgtatgtgtgtgctctatgtgtgcctatgtatgtgtgcatgcatgtgtgcatatacaaatgcatatatgtctatgtagtgtgcgtacacaaatgtatgtgtgtgctcaatgtgtgcctatgtgtgtgtatgcatgtgtgcgtacacaatgcatgtgtgtggtgtctgtgtgcctgtgtgtgtatgcatgtatgcatacacaaatgtatatgtgtggtgtgtgaatgtgtgcctatgtatgtgtgtgctgtgtgtgggtgtggtatgtgtgtgatgtgtggaggggtgtgtatgtgtggtatgtataggtgatacgtttggggtgtaatatgcgtatgtggtttgtgaaatgtagttcgtgtgtgtgcatgtgtgcgtgcgtgcgtgcgtgcgtgcgtgtgtgtgtgtgtgtgtgtgtgtgtgttggatatagtatgtgtgaggtgtgtgtactcaccatggcctccctcacttgggggagtgaagtcagcagcctggaccactcagggacatgagatactcagacacatcttgatttccacccctcttttcctgatcctccttcacgtgtcactttcccaaacactggacaacagtttgggggcatctgattccactaatgacagggacatcacatgtctccagagggaacaccttctgtgtcacatgtcatctgagaatgtagcagagtcacagagaaatgtcacagaaaccaaaatgcagagtaccaaggtatagctaggcacagagcagaggggaagccgctgaatttattaaaaatgtcagaatcgtaaaagacaggggacagcggtggggacattcagggtccagtagcacacaggcagtccaaacctgatcactggaaggtagtaggtaaggaaaggctgcacacagattattcacacagtttatacatgtacacagattattcacatggtttgtgtatgtgcacagattattcacacagtttatacatgtgtggcttcgtggtaactttgagcttactttcaatttaaaaggatctctctcacaagctggggccgggaatggctgcagtcaacactccatcacttagtcacactgtgcaaacagcacctcctgactcatggtgacttgtagtaaaatgaagaggccacatttgcatccaagacagctcatcagtacctagtgaagaatctgtccctgagtatttgcatgaatggacccgggtccagggcctggctgggagtctccaggtgttgcagccagaatgtcattgtgttttttcaggatcccagaagtttctaaaatacaggccaagtactcatttgtgttacaaagtatctgactaatagaagtgattaggtaacacaaagccttttaaaaaccgagatcacccttgtcatgtccctggcctcttagaacaagatccaagcttttgctggttgacaagtggggccatccagtgcgtctccgttcctgctacttcatctggaagacctctcccactaacttgcccctgacccctcacacctgctgtttcctttccacccggaagtgcttgtctaggctttcatggccatctgactgagcatctaggcctcagtccagtggtccctcagctctctctagtcactgtactaatggaaacggccactaactacattttcaatatggaagcctcctcctcaggaacctccaagggcagaagcctccagagaaccactcctgaccccctggagttctgagtgcttctggccctctctgtgtctgcaggactattcaccacttgtgttgaatggttcagtcctcacctcctctggcatgtgctcagttctcatctcattggggagtccttcccaggtcactcttctctcctgtctttgaagtgtttttttccttcatggtatttctgtctgggcacacacacagacacacatacacacacatacacacccatgcagtatggcagatacatcacctatgtttcagatttttattctaccatcacccaatacctgaatccccgaaaaagccttagaaagccaggaatttgtgtatttttgtcagcactccaccccagcacctgaagccaagcctgacttaatatttttggttttgtttctaga

SEQ ID NO: 14 - DNA, 7045 bp, Cricetulus griseus
  source 1..7045: mol_type = genomic DNA
caattgattatagatggatgatagatagatagatagatagatagatagatagatagatgatggatagacagatgatggatagttagaggatagataatgactgaataataagtacataaatagatgatagagcggggcgttggtggtgcacgtctttaaccccagcaccagagaggcagaggcagttggatctctgtgagtttgaggacagcctggttacagaatgggttccaggacagccaaggctgtcactcagagaaatactgtctcaaataaaaaaagtaagtaaacaaataaataaatgataactagttagaagatagatgattgaatgataggtagataaatagaagatagatagatagatgattgatagatgatagacagatagacagacagacagacagacagacagcagaaagataatgcacggtgaaacatggtctgatttagttagcaagatcagagaagccttctttgaaagtgacatttgagagcatttcaaacgctgttcatgtcaggcatgccaatggggagagaagggcttgcagaaagcaggcccggcaagccatggggagcaagctaggaggcagcattccttgcatttgcctctgcctcagctgcttcctggagttccccggtttttatcacaacagtagaaataaaaccaggacaatgttgtttccatgcatacatctgcaagaacttactccggttcaatagacagaccaaggcacctgtgtttgctcaagaagcacggagggaggtgtgtgcacctgctgggtgctggtgctctggctgtgccagacagagagcaagacaggaaagttcctggtggcctagagcacacagcccagcccaggaagtcatgtctctctctgtctctgtctctgccccacccccaccccatttaggccagagaacagctgtggcaagctttgggtttgggtgagtcattcctcaagagccaagagccgcccaccttgtatggggtagtttgttgttgttgttgttgttattatttgtttgtttgtttgtttggtaaaggtttttcaataggagttggaatttggcaattcagctaggctggctgagcagccagctagccccgggcactcatccgtctctacctccccagttctgggatttcgggtacatgctgccacatccgacttttttcccctgctccagttcttaagaccaagtcttcatgtcaaacacttcaccaccttagccatctttctgggtcagaagttagatcttcaggaagacaaggagtgtatcaggacatgagcgtgccccaactctgctcagaccttctgatagagaaaatggggggaggggtgtcagaggctgccggagaaagacaagtccaggttaaggaggacgaccctgggctctgaatccaagggtgattccctcaccttgtacacttggcattttgggaaggaagcatcagataaaagcagtgcagacatagtcaggaatatttacacgtgtgagtcaacctgggagtgagtctgtgtacaactgaacatgaagcaagttttgaagcttcatttccagactattcccagggtgcaataacttcctgttttcgttgcagccttcccagtctctgccactgccatctctacttcagtctggaatggtgggcacacagaaaaagtctatggcaatcctgcgagaagacaagtgggcgcctgacttcgggctcctgttacaagagaggaatccaggagtttattttgcagctgattcagtgttgaccaagagtccagctctgggggagtgggaagcaaccaaagcagagacaggtcccagcacaatttttggttttcaagacagcacttctctgtggctttgaaggctatcctagaactgttctttgtatatccttccttgcaactagctcttatagaccaggctggtcttgaactcacagagatccatctgcctctgcctcccaagtgctgggattaaaggcgtgcacctcggctgccaccacccagctacatacataatttacaataataaaaataaaatactttaaagtgttatagcagtttgaatgtaattggccctgtcatctcatagggagtggcactattaggaggtatggctttgttgaaggaaatatgtcactgtgagggtgggctgtgaggtttcctatgctcagggtaccagccagtgtctcagctgaggtcctgttgcctgcaagatgtaggactctcatccctttctccagcaccatgtctgtctgcatgccatcatgttcccagccatgatgacaatgtactaaaacctctgaaactgccacccaactaaatgttttcctttataagagttgccatgctcatggtgtctcttcacagcaatagaaaccctaactaagataagtgtattctcccctactccccatgatttaaaatttaggaaggcaggtaggcaggcaggcaggctggtatagtggttcattctagcacctgagacctggaatgggaggattgtgagttagttctaggccattctggtgcctagaaaccagagccgggggttggcccaatgcagagcacttgctctacgtatggcccagcacaataagtcaatttcctcaccttaaaggcttgacaatttaaaaacactggtttttagttagtccgtgtctgctccacagatggagacagctaatcacagatgcatcaggggccttcctgagtgctaaacatcaaacagccttctcccctcctgagcctttgtgtgcagaatgtgtccatcgcaagaagcaaacagtcttgcttgcccaccaacttccttcctgcatcagaagagctgggtgcaaactgcaagagtagcctcaccttagagatgggtcccattgctctacatgggagcattaccttccaagaaggcaaaaatgtctcctggttgagctttttttgtcacctgttaaaggcaaatcaacagagaggctttgtctcacccactaacatcttggaaacaaataccaacgaacgctggggaggatgtggggaaagcagagccctcatgctctccgagggaaaatcacacccactgtggaacagtgtggaaacctcaaagactgggattacaagcagcacacaagccagccacgctactcctggtcacacaccacaaagacgcttgcacattcacgcttacgctgcgaacactagcaacgttcccactgcctcctttgagccccgccccccgcccctgccccccgccccgcccctgtggtctatgttcctcttccctaaagtcagcttccacttctctgtctccatcttcgccccaccctccctcctcgctacataattgtctctattccatttctctgctttgaaacagctttttgcaaagcatcaaatctattgtcctatgccccaaatcaacctccagtttcacaagtgatacaggaaatcgttttcctaattaaaaatcccccctttgaccatttattcccactcttggaacatcttccccttgaggaaagttacagaatgaggtggctctcctcttcctattcgaggtgtttccttcagactttgtccgtgtctaatctttttaactgttggccaggcctccaccacggcacagatgaactgtggggttcatttacctgaaactctatggaaggatgtttatttctccttcactttagcaaatgataaagggcaccattcactctgtctattctgcaggggccattcctttctctaggccagatactgagaattgctcccagaatcaatgtggtatacatatttccccttcaacattgataggcattgatcacacacacacacacacacacacacacacacacacacagtagcacaaatgtattcccctagcccgcttccatcttgccacaggactccagagtggccctggatagcaagcttcctgttttgtttctctgttcctgctgcttttccaccctccagtctatcttttctaagtccttctgccattgtcctcttcccaactgtcctgagatgcagtcattgtctgggattcagaccttctctctctgcccaagtgagtatattgacccccacggtttgtacaaccataacttcagggagcccgacaaaaactgttttatgagccaagtagtcccaggacttgagaggtagaggcgggaagatcagcagtttgaggccagcctggagagcataagagccggtctcaaaacaacaatggaaactagatactaagtaaaaatcctggggtgtttcatcatgaatgtctgttcttctagtaccacgctgaactccgtacacagctccagctgttacggctttcttagaatccatactctttttttttttttttttttttttttttttttggtttttcgagacagggtttctctgtggctttggaggctgtcctggaactagctcttatagaccaggctggtctcgaactcacagagatccacctgcctctgcctccagagtgctgggattaaaggcgtgcgccaccaacacccggcagaatccatactctttttaaaaaaagatttatcaatttactatgtatacagctttctgcctgcatgtatccatgcatgtcagaagatggcaccaggtcgcattacagatggttgtgagccaccatgtggttgctgggaattgaactcagaatgtctagaagagcaaccagttctcttaacctctgagccatctctccggcccccagaaatccatattcttgaggattttttacaccccccccaccaaaagacgtatatctaaattttaatgtgagaattcacattttcttaagagttgaacatagatttagaggaaaatcagatcccacatgattaacaaagcatgcttgtgggcaggtctgctaccaagaggtgggccgtagcttctagctcagacaaactcactcccttcctcgtggcctcttcgccctcaagtcagaaactcaccctgtgattctgccccagaagttgctctagagcacagtgcatccttccgtcttcactctgtggcttgaattgtgtccatcgcttatgattacaacccctcacagagcatcctaactggtttctttgcatgcctatgggcactcctccattctagaacacccttgccatcaatactatgaaaggaggggtggaggaggaagagcaggaagaggagggggaagcgagggaagaggaagacacggatggcaatgaggaggggggagcacccaagtcctccctggatgagagtctcactgggagacttaatattaattataaatgcttggtcagcagctgggcaggataaggttaggcaggagaaccagactaaggactctgggaagcagaagggcagagtcagacaaggagaggaaacaggaagtacaaggtaaagtcacgtggcagaatgtagataatagaaatgggttcatttaagttggaagagttagctagtaacaagcctgagctatcagccgagcatttataattaatattgagcctccatattggttatctgggaattggcgggcagaaaaaaaaaagtctgcctacaagtcaatgtcatgtagctcccaaagccaaggtacctttgttcagtgcttgactgagccagcattataaattttctccagatgtaccgaatcacatttcatagcaacatgcagacatcaagttttccctgaagctctaaccagctggttgcatgctgtccggagtctcagctataacccagaagtgacctgggtcggggaagaggtggtactttgccttctttgcactctctgtgttgcctcacccattcagcttcaagcaatgtgactgcctgaccctgagggcgtttacaacgcctgacccacagaccacaagtcaaccagctggtgtgctcacgatacctagtctgaaccatagccctgctcccaccctgcctccatctccaccctttcttcactgctcatcacagctggctagcaaagactgcctcagacctgagcacaggctccactccacagccgtgactgttcgagccacttaaatcaaagagcgcttgtcttccgctcagtaaatctctcctcagctcactgatgacgttgactttctctagacagcacatttgggtttaagacactgctacttgagctcttcattcagttcctcagaatacctcatttgggtcagattcccaaagaggaagatagggttcctggcagacagacatgtctcattcctttgaaatccttcagagaaatgcagtgactatggcaccttcttaaaaagcacacacacaaataacacacacacacacacacacacacacacacacacacacacatatccccctcactgtcatccttgatatgtatatgatatatataaaatcattgttttatactgtgataattgattatgaataaaatttactaaaatgaacaattaaaattatggggggggctggagagatggctcatcagttaagagaacagttgctgctcttgcagaacacgagagttcagttcccagcacccacatcaggcagctcataaccatgtgtggtgtcagttccaggagatctggtgccctcttctggcctcctccagcacctgctacatgtggttcacacacacacacacacacacacacacacacacacacacacacacacaaataaatataaagattatttttttcaaaactgagttaaaaataggttctatctgattcatactaaggcttttcacagtggttaagtctattagatatgtctagccatatcctttctcccttctttcttgaggagaggcttttaaagctacaagttacagccttctttgcaaataagagtaccatttaacaggcctctgaccaatgagatgccagaatcggttgcccaggagcttcccaaacagtccattatagggaaaggtggtacaaaccagtagattaggcatgttccacttcctaagtgccgtgccaaataaggaaatggcctcaaatgtttgccttttatcttcacccacctctgaattgcacgctagt

SEQ ID NO: 15 - DNA, 13515 bp, Cricetulus griseus
  source 1..13515: mol_type = genomic DNA
tctagaaacaaaaccaaaaatattaagtcaggcttggcttcaggtgctggggtggagtgctgacaaaaatacacaaattcctggctttctaaggctttttcggggattcaggtattgggtgatggtagaataaaaatctgaaacataggtgatgtatctgccatactgcatgggtgtgtatgtgtgtgtatgtgtgtctgtgtgtgtgcccagacagaaataccatgaaggaaaaaaacacttcaaagacaggagagaagagtgacctgggaaggactccccaatgagatgagaactgagcacatgccagaggaggtgaggactgaaccattcaacacaagtggtgaatagtcctgcagacacagagagggccagaagcactcagaactccagggggtcaggagtggttctctggaggcttctgcccttggaggttcctgaggaggaggcttccatattgaaaatgtagttagtggccgtttccattagtacagtgactagagagagctgagggaccactggactgaggcctagatgctcagtcagatggccatgaaagcctagacaagcacttccgggtggaaaggaaacagcaggtgtgaggggtcaggggcaagttagtgggagaggtcttccagatgaagtagcaggaacggagacgcactggatggccccacttgtcaaccagcaaaagcttggatcttgttctaagaggccagggacatgacaagggtgatctcggtttttaaaaggctttgtgttacctaatcacttctattagtcagatactttgtaacacaaatgagtacttggcctgtattttagaaacttctgggatcctgaaaaaacacaatgacattctggctgcaacacctggagactcccagccaggccctggacccgggtccattcatgcaaatactcagggacagattcttcactaggtactgatgagctgtcttggatgcaaatgtggcctcttcattttactacaagtcaccatgagtcaggaggtgctgtttgcacagtgtgactaagtgatggagtgttgactgcagccattcccggccccagcttgtgagagagatccttttaaattgaaagtaagctcaaagttaccacgaagccacacatgtataaactgtgtgaataatctgtgcacatacacaaaccatgtgaataatctgtgtacatgtataaactgtgtgaataatctgtgtgcagcctttccttacctactaccttccagtgatcaggtttggactgcctgtgtgctactggaccctgaatgtccccaccgctgtcccctgtcttttacgattctgacatttttaataaattcagcggcttcccctctgctctgtgcctagctataccttggtactctgcattttggtttctgtgacatttctctgtgactctgctacattctcagatgacatgtgacacagaaggtgttccctctggagacatgtgatgtccctgtcattagtggaatcagatgcccccaaactgttgtccagtgtttgggaaagtgacacgtgaaggaggatcaggaaaagaggggtggaaatcaagatgtgtctgagtatctcatgtccctgagtggtccaggctgctgacttcactcccccaagtgagggaggccatggtgagtacacacacctcacacatactatatccaacacacacacacacacacacacacacacgcacgcacgcacgcacgcacgcacacatgcacacacacgaactacatttcacaaaccacatacgcatattacaccccaaacgtatcacctatacataccacacatacacacccctccacacatcacacacataccacacccacacacagcacacacatacataggcacacattcacacaccacacatatacatttgtgtatgcatacatgcatacacacacaggcacacagacaccacacacatgcattgtgtacgcacacatgcatacacacacataggcacacattgagcacacacatacatttgtgtacgcacactacatagacatatatgcatttgtatatgcacacatgcatgcacacatacataggcacacatagagcacacacatacatttgtgtatgcacacatgcacacaccaatcacatgggaagactcaggttcttcactaaggttcacatgaacttagcagttcctggttatctcgtgaaacttggaagattgctgtggagaagaggaagcgttggcttgagccctggcagcaattaaccccgcccagaagaagtaggtttaaaaatgagagggtctcaatgtggaacccgcagggcgccagttcagagaagagacctacccaagccaactgagagcaaaggcagagggatgaacctgggatgtagtttgaacctctgtaccagctgggcttcatgctattttgttatatctttattaaatattcttttagttttatgtgcgtgaataccttgcttgcataaatgtatgggcactgtatgtgttcttggtgccggtggaggccaggagagggcatggatcctccggagctggcgtttgagacagttgtgacccacagtgtggggtctgggaactgggtcttagtgttccgcaagtgcagctggggctcttaacctctgagccatccctccagcttcaagaaacttattttcttaggacatgggggaagggatccagggctttaggcttgtttgttcagcaaatactcttttcgtgtattttgaattttattttattttacttttttgggatagaatcacattctgcagctcaggctgggcctgaactcatcaaaatcctcctgtctcagtctaccaggtgataagattactgatgtgagcctggctttgacaagcactttagagtccccagcccttctggacacttgttccaagtataatatatatatatatatatatatatatatatatatatatatatattgtgtgtgtgtgtttgtgtgtgtatgagacacttgctctaagggtatcatatatatccttgatttgcttttaatttattttttaattaaaaatgattagctacatgtcacctgtatgcgtctgtatcatctatatatccttccttccttctctctctttctctcttcttcttctcacccccaagcatctattttcaaatccttgtgccgaggagatgccaagagtctcgttgggggagatggtgagggggcgatacaggggaagagcaggaggaaagggggacagactggtgtgggtctttggagagctcaggagaatagcagcgatcttccctgtccctggtgtcacctcttacagccaacaccattttgtggcctggcagaagagttgtcaagctggtcgcaggtctgccacacaaccccaatctggccccaagaaaaggcacctgtgtgtgactctggggttaaaggcgctgcctggtcgtctccagctggacttgaaactcccgtttaataaagagttctgcaaaataatacccgcagagtcacagtgccaggttcccgtgctttcctgaagcgccaggcacgggttccctaggaaatggggccttgcttgccaagctcccacggcttgccctgcaaacggcctgaatgatctggcactctgcgttgccactgggatgaaatggaaaaaagaaaaagaagaagtgtctctggaagcgggcgcgctcacacaaacccgcaacgattgtgtaaacactctccattgagaatctggagtgcggttgccctctactggggagctgaagacagctagtgggggcggggggaggaccgtgctagcatccttccacggtgctcgctggctgtggtgcatgccgggaaccgaaacgcggaactaaagtcaagtcttgctttggtggaactgacaatcaacgaaatcacttcgattgttttcctctttttactggaattcttggatttgatagatgggggaggatcagagggggaggggaggggcggggagacggagggaggaggggaggaggggaggaggggaggaggggaggaggggaagggatggaggaaaatactaacttttctaattcaacatgacaaagattcggagaaagtgcaccgctagtgaccgggaggaggaatgccctattgggcattatattccctgtcgtctaatggaatcaaactcttggttccagcaccaaggattctgagcctatcctattcaagacagtaactacagcccacacggaagaggctatacaactgaagaaataaaattttcactttatttcatttctgtgactgcatgttcacatgtagagagccacctgtgtctaggggctgatgtgctgggcagtagagttctgagcccgttaactggaacaacccagaactcccaccacagttagagcttgctgagagagggaggcccttggtgagatttctttgtgtatttatttagagacagggtctcatactgtagtccaagctagcctccagctcacagaaattctcctgttccggtttccaaagtactggagttatgagtgtgtgttaattgaacgctaagaatttgctgattgaagaaaacctcaagtgggtttggctaatccccacgaccccagaggctgaggcaggaggaatgagagaattcaaggtttgccagagccacagggtgagctcaatgtggagactgtgagggtgagctcaatgtggagactgtgagggtgagctcaatgtggagactgtgagggtgagctcaatgtggagactgtgagggtgagctcaatgtggagactgtgagggtgagctcaatgtggagacctgtatcaagataataatagtagtagtaacaatgcaggcgagggtgtggttgagtggtagagcagttagttgatttgacatgcttgaggtctcccggtccatctgtggccctgcaacaggaagggagggaggaagggggggaacgagagagaggaaagagagacagaagctaagatagggaatgagagaggaaggaagaaacgggaagaaattcagactccttcctgagttccgccaacgcctagtgacatcctgtgcacaccctaaggtggcctttgtgtggcactggcttgggtggtcgggaaaggcattttcagcttgttgcagaactgccacagtagcatgctgggtccgtgaaagtttctgcccgttaacaagaagtctctactacttgtgacctcaccagtgaaaatttctttaattgtctcctggtgttctgggttttgcatttttgtttctaaggatacattcctgggtgatgtcatgaagtccccaaagacacagtggggctgtgttggattgggaaagatgatttatctggggtgtcaaaaggaaaagaagggaaacaggcacttgggaaaatgtcctcccgcccacccgaattttggcttggcaaccgtggtggaggagcaagaaacacgtggacgtttgaggaggcatggggtcctaggaggacaggaagcagaaggagagagctgggctgacagcctgcaggcattgcacagtttcagaaggagattacagcatgactgagtttttagggatccaacagggacctgggtagagattctgtgggctctgaggcaacttgacctcagccagatggtatttgaataacctgctcttagagggaaaacagacatagcaaacagagccacgtttagtgatgaaactctcactttgcctgagtcatgtgcggccatgcccaggggtcaggctgacactcaactcaaaaacaagtgagaaattgaagacaatccgtggtggcagctactggaagggccaccacatccccagaaagagtggagctgctaaaaagccatttgtgataggcacagttatcttgaatgcatggagcagagattacggaaaaatcgagaatgttaatgaggcaacattcgagttgagtcattcagtgtgggaaacccagacgcttccatcccctaaaaggaacatcttgctctcagtcaaaatggaaataaaaattggggcttgaatttggcaaatgattcagaactctgtgtaggtattttcacacgcacagtggataattttcatgttggagtttatttgtgctaaaaggcagaaaagggtaaaaagcacatcttaagagttatgaggttctacgaataaaaataatgttacttacagctattccttaattagtacccccttccacctgtggtaatttcctgagatagtcagtggggaaaagatctctccttctcttctttctccccctcccctcctctccctccctccctccctccctccctcctctccctccctccccctttccttctttctttgctccttctcctctgcctccttctccctttcttcttcatttattctaagtagcttttaacagcacaccaattacctgtgtataacgggaaaacacaggctcaagcagcttagagaagattgatctgtgttcactagcgtgcaattcagaggtgggtgaagataaaaggcaaacatttgaggccatttccttatttggcacggcacttaggaagtggaacatgcctaatctactggtttgtaccacctttccctataatggactgtttgggaagctcctgggcaaccgattctggcatctcattggtcagaggcctgttaaatggtactcttatttgcaaagaaggctgtaacttgtagctttaaaagcctctcctcaagaaagaagggagaaaggatatggctagacatatctaatagacttaaccactgtgaaaagccttagtatgaatcagatagaacctatttttaactcagttttgaaaaaaataatctttatatttatttgtgtgtgtgtgtgtgtgtgtgtgtgtgtgtgtgtgtgtgtgtgtgtgaaccacatgtagcaggtgctggaggaggccagaagagggcaccagatctcctggaactgacaccacacatggttatgagctgcctgatgtgggtgctgggaactgaactctcgtgttctgcaagagcagcaactgttctcttaactgatgagccatctctccagccccccccataattttaattgttcattttagtaaattttattcataatcaattatcacagtataaaacaatgattttatatatatcatatacatatcaaggatgacagtgagggggatatgtgtgtgtgtgtgtgtgtgtgtgtgtgtgtgtgtgtgtgttatttgtgtgtgtgctttttaagaaggtgccatagtcactgcatttctctgaaggatttcaaaggaatgagacatgtctgtctgccaggaaccctatcttcctctttgggaatctgacccaaatgaggtattctgaggaactgaatgaagagctcaagtagcagtgtcttaaacccaaatgtgctgtctagagaaagtcaacgtcatcagtgagctgaggagagatttactgagcggaagacaagcgctctttgatttaagtggctcgaacagtcacggctgtggagtggagcctgtgctcaggtctgaggcagtctttgctagccagctgtgatgagcagtgaagaaagggtggagatggaggcagggtgggagcagggctatggttcagactaggtatcgtgagcacaccagctggttgacttgtggtctgtgggtcaggcgttgtaaacgccctcagggtcaggcagtcacattgcttgaagctgaatgggtgaggcaacacagagagtgcaaagaaggcaaagtaccacctcttccccgacccaggtcacttctgggttatagctgagactccggacagcatgcaaccagctggttagagcttcagggaaaacttgatgtctgcatgttgctatgaaatgtgattcggtacatctggagaaaatttataatgctggctcagtcaagcactgaacaaaggtaccttggctttgggagctacatgacattgacttgtaggcagacttttttttttctgcccgccaattcccagataaccaatatggaggctcaatattaattataaatgctcggctgatagctcaggcttgttactagctaactcttccaacttaaatgaacccatttctattatctacattctgccacgtgactttaccttgtacttcctgtttcctctccttgtctgactctgcccttctgcttcccagagtccttagtctggttctcctgcctaaccttatcctgcccagctgctgaccaagcatttataattaatattaagtctcccagtgagactctcatccagggaggacttgggtgctcccccctcctcattgccatccgtgtcttcctcttccctcgcttccccctcctcttcctgctcttcctcctccacccctcctttcatagtattgatggcaagggtgttctagaatggaggagtgcccataggcatgcaaagaaaccagttaggatgctctgtgaggggttgtaatcataagcgatggacacaattcaagccacagagtgaagacggaaggatgcactgtgctctagagcaacttctggggcagaatcacagggtgagtttctgacttgagggcgaagaggccacgaggaagggagtgagtttgtctgagctagaagctacggcccacctcttggtagcagacctgcccacaagcatgctttgttaatcatgtgggatctgattttcctctaaatctatgttcaactcttaagaaaatgtgaattctcacattaaaatttagatatacgtcttttggtggggggggtgtaaaaaatcctcaagaatatggatttctgggggccggagagatggctcagaggttaagagaactggttgctcttctagacattctgagttcaattcccagcaaccacatggtggctcacaaccatctgtaatgcgacctggtgccatcttctgacatgcatggatacatgcaggcagaaagctgtatacatagtaaattgataaatctttttttaaaaagagtatggattctgccgggtgttggtggcgcacgcctttaatcccagcactctggaggcagaggcaggtggatctctgtgagttcgagaccagcctggtctataagagctagttccaggacagcctccaaagccacagagaaaccctgtctcgaaaaaccaaaaaaaaaaaaaaaaaaaaaaaaaaaaaaagagtatggattctaagaaagccgtaacagctggagctgtgtacggagttcagcgtggtactagaagaacagacattcatgatgaaacaccccaggatttttacttagtatctagtttccattgttgttttgagaccggctcttatgctctccaggctggcctcaaactgctgatcttcccgcctctacctctcaagtcctgggactacttggctcataaaacagtttttgtcgggctccctgaagttatggttgtacaaaccgtgggggtcaatatactcacttgggcagagagagaaggtctgaatcccagacaatgactgcatctcaggacagttgggaagaggacaatggcagaaggacttagaaaagatagactggagggtggaaaagcagcaggaacagagaaacaaaacaggaagcttgctatccagggccactctggagtcctgtggcaagatggaagcgggctaggggaatacatttgtgctactgtgtgtgtgtgtgtgtgtgtgtgtgtgtgtgtgtgtgatcaatgcctatcaatgttgaaggggaaatatgtataccacattgattctgggagcaattctcagtatctggcctagagaaaggaatggcccctgcagaatagacagagtgaatggtgccctttatcatttgctaaagtgaaggagaaataaacatccttccatagagtttcaggtaaatgaaccccacagttcatctgtgccgtggtggaggcctggccaacagttaaaaagattagacacggacaaagtctgaaggaaacacctcgaataggaagaggagagccacctcattctgtaactttcctcaaggggaagatgttccaagagtgggaataaatggtcaaaggggggatttttaattaggaaaacgatttcctgtatcacttgtgaaactggaggttgatttggggcataggacaatagatttgatgctttgcaaaaagctgtttcaaagcagagaaatggaatagagacaattatgtagcgaggagggagggtggggcgaagatggagacagagaagtggaagctgactttagggaagaggaacatagaccacaggggcggggcggggggcaggggcggggggcggggctcaaaggaggcagtgggaacgttgctagtgttcgcagcgtaagcgtgaatgtgcaagcgtctttgtggtgtgtgaccaggagtagcgtggctggcttgtgtgctgcttgtaatcccagtctttgaggtttccacactgttccacagtgggtgtgattttccctcggagagcatgagggctctgctttccccacatcctccccagcgttcgttggtatttgtttccaagatgttagtgggtgagacaaagcctctctgttgatttgcctttaacaggtgacaaaaaaagctcaaccaggagacatttttgccttcttggaaggtaatgctcccatgtagagcaatgggacccatctctaaggtgaggctactcttgcagtttgcacccagctcttctgatgcaggaaggaagttggtgggcaagcaagactgtttgcttcttgcgatggacacattctgcacacaaaggctcaggaggggagaaggctgtttgatgtttagcactcaggaaggcccctgatgcatctgtgattagctgtctccatctgtggagcagacacggactaactaaaaaccagtgtttttaaattgtcaagcctttaaggtgaggaaattgacttattgtgctgggccatacgtagagcaagtgctctgcattgggccaacccccggctctggtttctaggcaccagaatggcctagaactaactcacaatcctcccattccaggtctcaggtgctagaatgaaccactataccagcctgcctgcctgcctacctgccttcctaaattttaaatcatggggagtaggggagaatacacttatcttagttagggtttctattgctgtgaagagacaccatgagcatggcaactcttataaaggaaaacatttagttgggtggcagtttcagaggttttagtacattgtcatcatggctgggaacatgatggcatgcagacagacatggtgctggagaaagggatgagagtcctacatcttgcaggcaacaggacctcagctgagacactggctggtaccctgagcataggaaacctcacagcccaccctcacagtgacatatttccttcaacaaagccatacctcctaatagtgccactccctatgagatgacagggccaattacattcaaactgctataacactttaaagtattttatttttattattgtaaattatgtatgtagctgggtggtggcagccgaggtgcacgcctttaatcccagcacttgggaggcagaggcagatggatctctgtgagttcaagaccagcctggtctataagagctagttgcaaggaaggatatacaaagaacagttctaggatagccttcaaagccacagagaagtgctgtcttgaaaaccaaaaattgtgctgggacctgtctctgctttggttgcttcccactcccccagagctggactcttggtcaacactgaatcagctgcaaaataaactcctggattcctctcttgtaacaggagcccgaagtcaggcgcccacttgtcttctcgcaggattgccatagactttttctgtgtgcccaccattccagactgaagtagagatggcagtggcagagactgggaaggctgcaacgaaaacaggaagttattgcaccctgggaatagtctggaaatgaagcttcaaaacttgcttcatgttcagttgtacacagactcactcccaggttgactcacacgtgtaaatattcctgactatgtctgcactgcttttatctgatgcttccttcccaaaatgccaagtgtacaaggtgagggaatcacccttggattcagagcccagggtcgtcctccttaacctggacttgtctttctccggcagcctctgacacccctccccccattttctctatcagaaggtctgagcagagttggggcacgctcatgtcctgatacactccttgtcttcctgaagatctaacttctgacccagaaagatggctaaggtggtgaagtgtttgacatgaagacttggtcttaagaactggagcaggggaaaaaagtcggatgtggcagcatgtacccgaaatcccagaactggggaggtagagacggatgagtgcccggggctagctggctgctcagccagcctagctgaattgccaaattccaactcctattgaaaaacctttaccaaacaaacaaacaaacaaataataacaacaacaacaacaacaaactaccccatacaaggtgggcggctcttggctcttgaggaatgactcacccaaacccaaagcttgccacagctgttctctggcctaaatggggtgggggtggggcagagacagagacagagagagacatgacttcctgggctgggctgtgtgctctaggccaccaggaactttcctgtcttgctctctgtctggcacagccagagcaccagcacccagcaggtgcacacacctccctccgtgcttcttgagcaaacacaggtgccttggtctgtctattgaaccggagtaagttcttgcagatgtatgcatggaaacaacattgtcctggttttatttctactgttgtgataaaaaccggggaactccaggaagcagctgaggcagaggcaaatgcaaggaatgctgcctcctagcttgctccccatggcttgccgggcctgctttctgcaagcccttctctccccattggcatgcctgacatgaacagcgtttgaaatgctctcaaatgtcactttcaaagaaggcttctctgatcttgctaactaaatcagaccatgtttcaccgtgcattatctttctgctgtctgtctgtctgtctgtctgtctatctgtctatcatctatcaatcatctatctatctatcttctatttatctacctatcattcaatcatctatcttctaactagttatcatttatttatttgtttacttactttttttatttgagacagtatttctctgagtgacagccttggctgtcctggaacccattctgtaaccaggctgtcctcaaactcacagagatccaactgcctctgcctctctggtgctggggttaaagacgtgcaccaccaacgccccgctctatcatctatttatgtacttattattcagtcattatctatcctctaactatccatcatctgtctatccatcatctatctatctatctatctatctatctatctatctatcatccatctataatcaattg

SEQ ID NO: 16 - DNA, 14553 bp, Mus musculus
  source 1..14553: mol_type = genomic DNA
cttgaagaacacatgttttccaagagggagcacccatgttggaatgacaatgtagttagtgctcctctcctgtaggttagtgctcctttgctataggtaagtgctcctctcctataggtcagtgctcctctcctataggttagtgctcctctcctataggttagtgctcctctcctacaggttagtgctcctctgctctaggttagtcctgctctcctatagtacctagagagctagggcaaatgggctaggcccgaagtgcagagacaaacagctatggaagactgggtaagcacttccaagctacgaaagagcagtgtgaagggtcagggcttgtgcagttagtaggggagatcttccagttgaagaaacagaagaactgagagccactgggtatcatcctcctgcgccatgccttcctggatactgccatgctcccaccttgatgataatggaatgaacctctgaacctgtaagccagccccaatgaaatattgtttttatgagagttgccttggtcatgctgtctgttcacagcagtaaaaccctaaataaggcagaagttggtaccagtattgctgtgatagacctgaccatgctttcctttgaaagaatgtggatttggtgactttggatttgcaacacagtggaatgctttaaatggagattaatgggtcatcaattcctagtaggaatatggaagactttgttgctgggagtatttgaactgtgttgacctggcctaagagatttcaaaggagaagaatttcagaatgtggcataaagacagtttttgtggtattttggtgaagaatgtggctactttttgcccttgtctgaaaagtctgcctgagactaaagtgaagagaatcagattaattgcattgacaagggaagtttgtggctgcgctatctggaaacttacagccagcctcttggacctcgggtgacttacgcaaatactcagggacagagatgcttgactctgtactgatgagttgtcttggatgcaaatatgggctcttcatttgactacatgtcacgatgagtcaggagctgctctctccagagtgtgacaaagcgaggggatgctgacggtagctgttctagctttgaaggtaagcctgcacttatgctaaagtcacacatacacgagccgggtggagaacctgtctgtgtggagacacctttcattacctgtggcatccagcctctcaagcttggactgcctgtgtgctcctggactctggaggtcccactgctctgtcctctgctgcttatgatactgacattttaaaagaatccagtggttcccccctgtactcggtgtctacttctacctggatgttcctcatttatgttctgtgacacttctctgtgactctgctgcattcctgggtgacatgtggacaccctgtccctttgcagaccatgatgtcactgtcactagtggaatcagatgccccaagtgttgtcctgtgtttgggaacgtgacaggcagtacagaagcagaagaggaagggtgaaaacggaaatgtcacagcagcatctgatgtgtgcctcagtcacgcatgctgctgattggaactactcagcatgagagagggccatggtgaatacacaaccctatacacactgtgtccatttctctctctctcttacacagagagagagggaggagggggagggggaggcggagggggagggggagggagagggagtgggagagggagagggagagggagagggagagggagagggagagggagagggagagtttaatgtctgtgaagagataccatgaccaaagcaactcttataaaggacaacatttaattggggctggcttacaggttcagaaattcagtccattctcaccatggtgggaagcatgcaggtagatgtggtgctggaggaaccaagagttctatatcctgatctgaaggcagccaggagaagactgcctcttctgcacagggcagagcttgagcatagaacatcaaagcccttccccacacttcctccaacaaggtcatacatacttcaacaaagacacacctcctaacggtgccactccctgtggaccaaccatttaaacgcatgagtctatgagggtcaaagctcttcaaaccaccacactcatgtacacacacacacacacacacacacactctcatacacacacacacacacactcacacacacacacacacacacacacacacacacaccacacacacacacacacacagagttctattttgcactgtttcactgtcacaaggttctacttatctcagacacactgccaggaattgtgtgggaagactttcagtttctttgggttcacatggacttagcagttcttggtgatcctgaaagatttctgcagaaagaagccaaagtgttgagcccaaggcctggccacacattagtcctgtctagatgaacaggggtttaaaaataagggggcatcaaggtgaagccagcaggggctgacttagagaggagacccacccaagccaactgctcgaagtcaaaagcgatgaatccccatatccagctgtgcccggtgctgtcttgctacatctttagtaaatgttcttttagttgtatgcgtatgaatattttgcttgcatatatttgtgtacaccataggtgttcctagggcctatggaggccagaagagggcatcagatcctttggaactggaattatagacacttgttacccatagagtagattgtgggaaatgagcctttagtcttcgagagcggccagtgctcttaacctttggtcgtttctccaggtctttgagactttattttcttggacatcaggacaggatccagggctttgagcttgtttcttcagccagctttcttttcatgtatattaaattttatgttattttgctttctttttccccaagacagaatcacactctatatagctcaggctgggtttgaattcagtttccctgtctcagtctaccgggtaatatgattacagatgtgagtctgactttggtatcaaagtccccagcccttctggatatgtgttttaaggatatcagatatatccttgatttgctttgaattttctttttagttacaacataattagttccgtgtcacctgaatatgtgtatgtcacctacatagtcttccttcttctcttcttccctctcccaccttcccaggtacctgtctgtcttcatatccttgtgctgagagtcttgttgagggagatgatgaccgagacagagccactggggaagggagatgggctagtgcaggtcttcagagaggagctcgtgaatattgtagcccctttagtccctggcatgtcctcttgtatagccaccgccatgctgtggcctggcagaagtgaataagttgtccagctgttgacaggcctgccctccagacccagtctgatcccaagaaagggcatctgtgtctgtctctgaggccgtaagtgctgcctggttgtctccagcttgacttgacactccctccttaataagagtaccacagaacagggtctgcagagtccctgggccaggtccctgtgctgtcctggaatgccaggcgtgaatttcctgtgaagtaggactttgctcgccaagctcccacggcttgcccttcagatagccagaattatctggtaccctgcattgccgttcaatacgcagagtatcactggaagcgcgcgcgcgcacacacacacacacacacacacacacacacacacacacacacgcccactccatctttaaaccccaccccccagcaacggcggtgtaaacactctccatcaggaagctgaaacgcagttgccctctgctggggagatgaaggcagcttgctgggggcgaggaccgtgctagcaaccttccctggtgcacacgggctctggtgcatgacgggaacggaaacgcggaactaaagtcagtcctgctttttttttttttttttttttttttttttttttttttttttttttttggcgttggtggtggactgagtgacaatcagtgaaatcacttaggttgtttttctcttcttcgttgggtttgatagacggtgggagagggtcagaggagaaggggagggatggggagagagggaggagggaggggcgggaggcggggggcgaggaaaacgtgctaacttctccaatcctacaagacaaaggtttggagaaagccgcactgagtgacccagcagaaggaatccaggaatgtccgctggaatctgactgttgattccagcgccatgcagagaatctaggctggtaggaacattctttgtcctatccgacataataactccaaccaacacggaaaagaaaggctatacaagtgaagaaatggcattttcactttcatgactatacaatcacttccaggtagtaacacgtgtctagcacagcggttctcaacctgggggtcacgatcccccacttttctgcatatcagacatttttacgttgttattcataacagtagcaaaattgcagctatgaagtaacaatgaaatgcatttatggtgcgtgtgtgtgtgtgtgggggggtatcaccttaacatttactgtaagaaggttgagaatactgctccagcagctagtgtgttggacttaggttctgggtatattattagcaatagccaaccagaatccccacccaccacagcattgaggccccatgcagggcttgctgggagaggcactgataagacttctttatgtatttatttagagacgaatactcattaggtaggccaagctagcgtcaaactcatggcaattctcctcctccagtttcctaagtactggactcaggagtgtgttgccatcatatacagtaaggatttattgactgaagaaaatctcaagtggctttggttaatccctactacgccagaggctgaggcaggaggcgcgcaaggtcaaggcttgcctgggctacatatagagtgagctcaattttgacacttggtgcggtgttagtagtaatagtaaagatgaaggtgtggctcaggtggggccggtgattggacacacttggggtctcctggtccatctgcagctgtgcaacaggaagagcggagaatgagaggaaagagagaaaagacagaatgagagagagggaggaagagagaaaaaggaaaagagagaggaaaggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaaaggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaatggaaaatgaggaaagcgagaaagaagaaatgagaaagaggaaagggagaaagaaatgagcgagataaaagacagaatttgagagagggaggaagaaataggaaaagagaggaaaggatggagaaaagagagaaagaaagagagatgaaagagagaaaggagaaatgaaatgagagagagagagagacacaaagagccagagagagaagaaaaaaggggaaagagaaagagaaagaggaaggctcctcttggacacatcttcctttatctttccctggggaccgccaaagcctggtggcatactgtacattctgtacactgttcattcaaaacaggctctgtcttaaagatggtctgagcggtcagaaaagggtattgttaacttgtttgcaaaactgcctcaggagagtgctgagtgcgtgaaagttgctgcccgttaaggagaagtctctactacttgtgatctcaccatcgaaaatttctttaattgtctcctggtgttctgggttttgcagttttgtttctaaggatacattcttgggtgatgtcacaaagtccccaaagacacggtggagctgtgttagatggggaaagacagtctgctgaggatttatctggaactgtcagaaggaaaagaaggtaaatggggcacttgggaaagtggcctctagtttgacttctggcttagcaaaggttgtggggagataaggcatacacagtagttagcaggaggcaacagggtcctgggaggacgcgaggcagaaggagaggctgggctgacagcatgcaatcattgcatagtctccaaaggagattgcaacatggctgagttttcagaggtcctacagagcccgtggtagagattctgtgggttctgagacaacttgactttagccagatggtatttgagtaatctgggagagagaaaacagctacagcaaacagggccacatttagtgacgaaactctcactttgactgttgagtcatttgcagtgggccctgaggtcaggctggccctcagctcaaaaacaagcgaggaactgaagcaattactcagataatccacagccacagccactggaaagggccacatccccagagacagcacagcaggggtgggggtggggctatgagaaagttagtgattgtagcagttatctagaatgtgcggagcagaggaggttacacaaaaacctagaatgtcattcaatgtgggaaaccgagaggctcccaagccctaaaaggaacagtttgctttcagccaaaatggaaataaaatttggggcttaaatctggcaaatgattcagaccttctgtgtaggtgtctttaaatgcacagcagattgattttcatgttggagtttatttgaactaaaagacagaaatggtgaaaagcacacctgaagaaattgagatgctatgaataaaatcatttacttacagctatcacttaattagtacctccttccaccttgctgatttattgggctagtcaaggaagaaaagatcttccctcctccttctctcctcctccccctcctctcctcctcccctcccctccttgaccttcctctcctccttttccctcctccccctcttcttctcttcaccccctcctcccctcccctcctctgtactcctcccctttcctcccaatctcttttttctcccccttcttctctttctcccccctcctcttccctcctcttcctccctccctccctcctcctcctcatcctcctcttcctcttcatcctcttctccttcctccctctcctcctcctccttttccagccctacctaccttccctttcttcttcatttattcaaagtagctttgaacagcactactcggtttagttgtgtataaaaggaaaatgcaggtccaagcagcttggggaagattgctttttgctctctggaggcagatgatgacagttcaagatcattccttttgctccatgtcacaggaagggggacatgccgaatctaccagtttgcagccacctacacaggatccaccttcacttctaaggaaatgtttgggaagctacctaccaaccacttctggcatctcatgggctagaggactcttaaatggcactcttatttgtttaataaaggaggttgtgacgtgtagttttaaatcccttccacacaacaattgctactctctgaccaaaaaagaagggagacaggatacggctaggtgtctagtagactttaccactttgaaaagccttaatataaatcaggtagatacatctttttaacttattcttgtaaagacaaaaacaaaactttatttttatttgtgtgtatgcttgtgtgtgtgtgcctgtgtgtataccacatgtcgctggtgccggagaacaccagaagaggggacctgatctcctggagctaaagctatccatggttctgagctgcctgatgtgggtgctgggaacagaactctggtcttctgcaagagcaacaagcctcctcttaactacgaatctcctccccatccccccaaatacatttaattattcattttagcagctttatttcgtaactacttatcacagcataaaacaaggattttatatatattacatgcaatcgaggataagagttgaggggagatgcgtgtgctccttctgggtgtctgtgcttttgaagaatgtaagcagtgcacaagggaccgaggcgtgcctgtctgccaggagctgtcttcttcccttggactctgagctgagtgcagtgctccgaagaagtaaaagacgacctcatgaagcaatgtcttcaacccaaacatgctgtccagacaaagtccagcttcattagtgctctgaggagagacttactgagcctcaggaaagcccccctcagcatggcgaaagtccactttgattgaagtgactcgaaagccatggcagtgcggcggcggccgcgtggagcttgtgctcgagtcggaagcggcatctttgtcaggcggctgtgattagcacggggaggcaggactggagtgaaggaagagttgggggcggggcttagcgctctggtctcctaagctgtagtcagcgcctcaagatttgtaacctgccttctgccttcccagccaggcagtcaagtggctccaagctgaagactgcaaagtgcccctaaccttttggttatagcgaggctgaagacaccgtgctctttcatgaaagccggatgtctgaaatccgatttgataaatatggataaaacgtataacgctcgatcaatcgaatcgaaggagctcacgattggcaccacggctttggggacaacagagtactgactcgttgggaggacttggatacttcccctcctcttccatctcttcccctttcctcacttcctcctccttccttctccattttctccctcttcactgtttcttactatttttacaaaagattttatttatttatttatttatttatttatttatttatttatttatttatttatttaatgtatgcgagtacactgtagctgtcttcagacacaccagaagagggcgtcaagttccattagagatggtttcgagccaccatgtggttgctggggcctctggaaggaccgccagtgctcttaacccctgagccatttctccagtacccttctcaccgtttctcttcaatcttcttcctcttccttctccactttccttgtcttcttggtttcattatctttctccctttcttcctcttctccccttcttcctcctccactgtagttttccttccctactcttttcctgcctccctcctcctcccctctcattccccctcctctttcctccttctccctcctcctccttccttctccctctcccctctcccctctcccttctcccttctccccctcctcttcctctttctccttctccacccctcctgtcacagtatcaatggcaagggtgttctagaatggaggagtgtcccctaggcactaacgaaagccagttaggatgctctgagacgggtacaattcagggagggccgtggggatggaagggttgtgctgcgattcattctggagcaacccccaggcagaatcatgaggttggttccggattcgcagggcacaattcagaagaggaaggtttcaggaaggacgagtttgtctgagataggagttacatctgatgtcttggcagcagagccactgtacaagcgtgctttattaaccacgtgggattaaatcttcttttaaatttattttcaactcttaaggaaacgtgaactttcacattcaaatttagacttgcagctcttatggggaaaaaaaggggatcttaagaatattaagcataggcggctggagagatggctcagcggttaagagcactctctgctctcccagaggtcctgagttcaattcctagcaaccacataatagttaacaacagtctttaatgaattctaatgccctcttctggtgtgtctgaagacagttacagtgtactcatataaataaaataaagaaatttaaaaaaatgaatattaggcatagattcctggatcctaagaaagccatcagagctggagccatgtgtgggatcctgcttggtgctggaggggcagagttcatgcccccggggtttttacttattatcacattttcatcgttgttttgaaacagggtcttgtgtggtccaggctggccttgaactcatctttcagcctctacctcacaggttctgggattacttggttcctaaaagtatctccgtcaagctccctggtgttatggctgtgccaaccaggagggtctatacactcgctcaggtagagggagaagatccgaatctctgacagggactgctgcctctcggggcaaatggagtgaaggacagcggcagaaggatttaggaaagatggacgggagagtggaaatgctgcagaagccagaaaacaaagcaggaagcctgctgtccagtggggctcaagagcggagggatgcgagggggctgcgcaggaacatttagcgtctgcgtctatgggggtaggggcggggtgccagcacctagtcacctgaaggggaaatgcttgcccagggagcaggtctcagtagctgacctagagaaaggagcggcccctacagaggagacacgggtcactgtttgttaaagtgaaggagaaataaatattctttcaaagaatcttaggtgagcccagttcatctgcgctgtggaggcctggggaacagttaaaaagaccctgacacacacccaaggcaaacaagcaacacacggctccttccgtaagggtccatgattctctgaagaatcagccccggaatcagccccggaatcaggtagtccgtaaacacaatgagtgttttactctgcagaagtccagcctgctggcgtctcccattaccaaaatagagggatagtcacgtgagctcaccggctcgatttaaggcacgtggttttccagggtagatgagctttggcttctggaaccattatggggcacgaaggatggagccaggattttttttttttttttttttttctattagcaattgatttgcttgggcttggctggacttgcccagttcttaggcccagtcttcttaactgccgatctgaagtctgtcatggagtcagcctagccttctcacttcccttcagctcgaataggaagaggaggtgcacaccagatggtctgagagcagggataaatggtgtgcctttgtctttcagtatttcgttattttaagtaggaagatgcttttctgtattacattgcttgtgaaaccggaagttgattcggggcacaggacaatggatttggtgttttgcaaggactgtttcagaagagagaggagtggaagggtggttagagtgaggagtggggtgggacgggatgggggaagagaaggaagggccagacaggctaggtagggctgagaggaggcggtgggaacttcttgagttagcgcagcagtaaacttggatgtgcgtgtatctttgtgatatatgacccggagccgtgtagctggctccgatagtactgctaatgtcagtgtcggggggggggggtcccatactgttccacaggggctgcacattcccatcgagagcaggagggctcctctctccatacatcctcgccagcattccttgttgtttctgtgatgacagggggtgggatgaaatctctctgttggtttgagagaccgtgaagaagctcaaccccaggacattttgcagtcttggaaggcagtgcctccatgtggagccgtggagcccatctctgagtccaggtcactcttgcagttcgcactcagctcttcagatgcaggagagacgttggtgggaaagcaagattgtttgcttgttgagatagacacattctccacacaaaggctcacgtggggcaaaggctgattgacgtacagcgttcaggaacgcctgtggtagagctatgattagctgtctccatctatgaagcagacaaagagttataaaaaaaatcaatgttttcaaattgtcaaacttttaacccgacagcaagcgctctgtccctgggctaatccctagccctggtttcttgagatggggtcttttgtgcactagactggcctagaactcacgatcttagtgttccagcctcccagctgctgggatgagccgctataaccagtctgcctgccttcctaaattttaagtgatgggaagtgggggagaatacagtttaaagtatgcagatctgagagcaggaacctggcaaagccaaggggccggagttacaggcggctaacatgggtgctgggaactgacccaggtccttgagaggagcagtgtgtactcttgaccaaacaggtccgtctctccagtccccgtagtattaaaaataggtactacgggcatggtggtgcacacctttaatcccagcactagggaggcagaggcaggtggatttctgagtttgaggccagcctggtctacaaaatgagttccaggacagccacggctatacagagaaaccctgtcttgaaaacaaaacaacaacaaaataggtactacaaagcgatgtaattgtgctcaaacatgcaaaccgaggggactgtatgcataagaaagagaaagacggccacactggttctatctgggtgacaggaaatcagtatttttatttttcacattcatttttttgttgttgttgttgacacagtgatttttctatcaaaaacattatttcttttatagttcccctgaggagctgtttttaaagccgtgctttgaaaaaccattgaaggagcagaggcagggagactcctgtgtggcagtcggtgaagcaggccctctgcaggcaggctggccctggacttgggagtctctttccctccctcctgtgctcaaatagcaaatgtcaggcttcaatgtagctagaaggttctagaatgattaagtttccaaggctgaagagcttccctgtttgcctttcacttccctggagaggtcgttgtgtgttccggagtctgcaaggtgcctttggtgatgcgggtggttcatctcgggagattccgcctggaggacccaagttcaagccctgcctgagctacagagtgactttcaggtcttctgcgcaattcagtgagacccagtctacaaataaaaagtaaaaagaaggctgtggatggaactcggtggtagagttctgggtttactccctagaggaggggagaaggaggaggagggaggaggaagaggaagaaagaagaagagaagggaagaggagaaggaagggagggaaggggctgacaagaagagagaagagggagggaggggagggaaaggaaggggaaaggaagggagggaaggggctgacaagaagagagaagagggagggaggggagggaaaggaaggggaaagaagagaagggtaagaagaaactgttccaatggtctgggccacagagtgatggccttttgtggtgatcagctgtaatccttgatttgacacaacctagaatctgggaagcgagtttctgtgaaggagcattcacactggctggcctgtgggcgtgcatgtgggagactgtcataattaggttcattaatacaggaagtcccagcccactacaaatggcttcgttccatacccaagagatgctaactgtagacggttggagaaagcaagcaagctgtggataccccacgctctttcacctcggctcctggggggtgggtgcactgtgtctcttggtattttaaagtcctgccttgacgtccctgctgtgacagactgtaactggaattgtgagctttagtcctttagttttctacgttggtttttctcaggatattttatcgcagtaacagaaacaagaccaggacacttgatctcctctgatcaacactgaagagttacaaaacaggctgaggaaacaaactttcttctccctctcccccttctgtccctccccttccttctcgctccctcccttgccccctctctccctgtctctgtctctgtctctgtctctgtctctgtctctgtctctgcctctcccctcccctcccctccctctgtctctgtctctgtctctgtctctgtctctgtctctgtctctgtccctttctcctctatctcctaaatggctggaggccatgctagctcaatgttgaactttgaacacgtatttaggaaatctttgttcttaacagttctgaagtgctgaagtggtggtttagtctctcggcctgacaagctcacttcctctcactctgtcttaatgaccaaatctgccatttccctaaaacagcacaggctccagctccaggttgctccggagcggag

SEQ ID NO: 17 - DNA, 4001 bp, Cricetulus griseus
  source 1..4001: mol_type = genomic DNA
ccaagatgcccatcaactgattaatagatgataaaattattgtacatttcagtgtaatattattcagtttttaagaaaaatgaaattatgtaataagcatgtaaatggatatatcttgaaacaaccattccccattatattacctaaacattgaaagtccaaaatcatatgatctttttagtggatctactaatcttttgctatatgtattttattgaactacccatggatgtgagataattggtaacaacagcacatgggagagcatgggatcattcaaggaagattagagagaatgcattttttaggagataatggaggagcaatagaaaggattaaatgaggttactgatgaaagtgatggttagagaaggcaatatgaggagggataactagcacttagggccttttgaaaaagacatagagaaaatactattgtagaaacttcctataattggtgtatagttatatacaccaaagagctcagatggagttaccctataatggaaatattaactactttttatcactgtgataaaacatcctgaacagagcaacatagattgggaagcatttactttggcttacagttctaacgggataaaaattcatgatgaaagaatgaatatgtcagcaaacagcagtagcaatggcctgagaagcaggtgagagctcacatcttgaagtgtaagaatgtagcagagagaacaaactgcaaatgaccagaaaatgcttttggatcagagcccatacccctctgactgacttctccagaaattctgaacaaataaaactccccaaacagagccataactgaaggtccagtgtctgagactactaggggtatttcttattcaaaccactacaatggggtggggggagcaatcctccaagtaggcactacacacagacaaataaaaactctagtaactggaatggattgacttatttgaattacttgccagtggagctacatagagcacaattattgtatttaaattaccctttatgatcttacaaaacttgacagtaagatcatattgctaaagaaaccacatatttgaatcagggaacatggtgatatctagttgttcttcaactggaaacttcatgctttctgcccagcattcatgttgctggaaagagcaatgtacactaccagtgtagaaattaaatcatcaatcttatcaagatgtggatcctataagttacaataaaaattagcctgataagatatccccaccagaagaatattcacataaatgctatgggagcaacaagctattttctaaattagctttaatcctattctacaagagagaatccatatctagaatagttatagggatcaagaacccatggcttgattggtcataggcccaatgggagatcctaatattattgttctacaaaatgaaaataactcctaatgacttgttgctgcagtaataagttagtatgttgctcaactctcacaagagaagttttgtcttacaataaatggcaattaaagcagccccacaagatttatatcataccgatctcctcatggcctatgcatctagaagctaggaaacaaagaggaccctaagagagacatacatggtccccctggagaaggggaagggggcaagacctccaaagctaattgggagcatgggggaggggagagggagttagaagaaagagaaggggataaaaggagggagaggaggacaagagagagaaggaagatctagtcaagagaagatagaggagagcaagaaaagagataccatagtagagggagccttgtatgtttaaatagaaaactggcactagggaattgtccaaagatccacaaggtccaactaataatctaagcaatagtcgagaggctaccttaaaagcctttctctgataatgagattgatgactaccttatataccatcctagagccttcatccagtagctgatggaagcagaagcagacatctacagctaaacactgagctagttgcagacagggaggagtgatgagcaaagtcaagaccaggctggagaaacacacagaaacagcagacctgaaaaaaatgttgcacatggaccccagactgatagctgggagtccagcataggacttttctagaaaccctgaatgaggatatcagtttggaggtctggttaatctatggggacactggtagtggatcaatatttatccctagttcatgactggaatttgggtacccattccacatggaggaattctctgtcagcctagacacatgggggaggttctaggtcctgctccaaataatgtgttagactttgaagaactcccttgagaagactcaccctccctggggagcagaaaggggatgggatgagggttggtgagggacaggagaggaggggagggtgagggaactgggattgacaagtaaatgatgcttgtttctaatttaaatgaataaaggaaaagtaaaagaagaaaagaaaacaggccaaaagattataaaagacagaggtggtgggtgactataaagaaacactattatctaaataaaaacatgtcagaagcacacatgaacttatagtgtttatgaaagtatgtataataactacataatctcaagccaagaaaaaaatatcatctttcagtgatgaaggtgattttatttctcccagaattaaagccaaagacctaatgaaagtaattatcttcaaaaggttgaaaatacatactttgcaatacacagatctgcctagaaatctcatgttcacaatacacatgatgctcaattgaattccattcaatgttacagtttagataaacagtttgtagataaactcacaatgtatcatttctttttattttttgaccaaacagcttctcatctgttattcagaataattcctcgatggcaggatatccatcccaattgggggaaggggagaatttgaagaaaacctagaccacatacatatttgccattgggaaacaaagtctaaaatgatgttgttcacatcttctctactagtcctctccccgtcccaaagaaccttggtatatgtgcctcattttacagagagaggaaagcaggaactgagcatcccttacttgccatcctcaacccaaaatttgcatcattgctcagctctgcccttctcatatgacagttacaagtcaaggcttccaaagtccctctgtcatgtttggtgtcaatagtttatacagatgacttcatgtcttcatatctaatgtcttatatagattaatattaaacaatgttatttctctaaccacattttaaattaatttaaaaatccattaattgtgtctataaaatgcagacagagtgctgagacacaatataagcctgatgatctgaatttgaaactcacacccaccacatggagaatcaacttccaaaaattttcctattacttccacacttacaccattgtacaaacacaataataatgaacaaaatgaaatgaaataaaaaattaagtctctgtaggtaatgctactgtgcagcaaaagtaaaaatggcagcttaagcttgctttatggttacactttaccatcttccattaattataaggacttcaatcatggcagaactatgctgttattgtctcagtgtaacctaaccaggtgttccagatgttcttaatgtggacacctaaactatttgatatttgggttaagatctttccctctttcagaagaaacctcaggacagagggaatcttgtcttttaattttgagtctgtagactttttccatttcaaatatacatgaaacaagtgatgaagaaaattaatcaaaaggtgggaattgcaatgatattaggttcaatattaagcttcaatattatcatggaatcgcctgttatacactgagtgtttggcaataagggatttttagaagaaggagtttttattctcaacaggttccttaagtttagctcaaataaatctaagcaatccactctagaattaaatagtttcc

SEQ ID NO: 18 - DNA, 14931 bp, Cricetulus griseus
  source 1..14931: mol_type = genomic DNA
catgtacacttatgcaagtatgatatggcccaacacagtattttacaccaatttttatctataaaatatacatgtacatcaaaatatattattaataataacatcattattctttctttccaagtaataaacacatacactgaaattttggttcttgtggataattttaatgaaacaggaaatgcaaatttatcttagcatgtttacttcactttctttgcatagataaccagtaatcacattgatggatcatgtagtgaaatgtatttttaggtatctaaggaattttggcttcgttttgtgcttgttgacactgaattctattcctaacaacagtgtgtaaggattctgtctgatttcttttaccagtatttgtccatttgcattttctttattattcatggctgctgttctagaaagtggaaggtagtgtgtcaagtctgtttaacatgtttccctgatgatcagtgtcttaacacctctctgagtacatgttggccaatgtcgtttctagacccatctattcttgcttgacttatcctggtacatgcctgccaagaaatttctcctcatcctttctgtctcttcactgatttacttgatgtgtggatttcacattgatcatatggaaatagaagatacaattttctttattcacagtttggaagactttcaatctcatagatcatcattattttttgctactgttccctatgctatggtgaaatttccatttgaataattgcttaaacaattaacaagaaagaatctatttttacttgcaataacttccatttcagaacatttactacactgttactatatccaaaaactagttttatatatcatgtgagaaatgactaattcataatttggccatgacatttttttcagaaacagaaaaagtgaccaatacatacacaatgctataaatattaagacttcagcaaattaaatatttattcatgatatcacataaaattcatttattatgttttatttaaatgtgtttttaaaacagtggtatcactaaatattaagttagatgtgtttatgtgcttaatgaatttatattttagaatgttataagttgtatatagtcaaatatgtaataaattttattttttaggtctttctcattaaggtattttaattttgggtcccttttccagagtgactctagctcatgatgagttgacataaaaactaaacagtacaaaatgtacattgcattcagtattgcacttgatctttgcactgaagtttgagtcagttcatacatttagtacttgggaagtacattaagctaactttcattgctctggcaaaatgctcgataagataagagtctattgtggaaagccatggcagcaggaaagtaagactgctgatgatgtttaatccatagtcaagacgcagaaggagatgaatgctggtatccaacattttttgctgttcattttctctagaaccctagtccataaagatgtatgacttgcattcaaaatgcgtccccttcagttgttcaacttttctgtaaatatcctttcaggcatgtctagaagattgtttcgcaaatacttctcaatccattcaagttgatagtgcagattaatcactgcagaataaaagcctgtaacttggctcacgtgccaaggaatatgcacactcctgacacatcaataagtaaatcaaagtgtagcttttgcctttaacattgccagacttatgtaatgttctgcacgttcttcctccatcactttttattctaatggtgtttccttgacattgaatcacgctgtggaagctgcttagaattaacattgaaatctactgatatatttatgatgcagcaatttagatttactattttacttagaattttttataattgagagaatataatattttcacagttatctatctgctgtaaatagaggattttaaaaaaaatctctataacttttttttacaacacacagtaaaattaagttaaaatttaataaagtcactatgttgatttcaaagtgtgctacgcccacggtggtcacgcaggtgtagcagaagatgccactaaggtgggctaaggccgatgggttggggtctgcgctccctggagatgagccccaggcggttccctggcaatcagctgcgatcatgatgcccgatgagccannnnnnnnnnnnnnnnnnnnnnnnnnnnnnnnnnnnnnnnnnnnnnnnnnnnnnnnnnnnnnnnctgggtgactttatggaaagaatttgatagatttcatgatgtagaagaattttattaggcttattttacaggagactaagaccctgggacctaaagatatctgggtcctgagaatcaggaaatgggtagagacgtggttgatggtatgagacagattttagagaactcttagatcatgggcaatgaccgcaatctgatgcttagaatagatcatctataaacaattatgctgttctttttctttctgttgtatgatctgatgatgtagcccccttgccaagttccctgatcccccttgccaagttccctgattgtaacagtatataagcattgcttgagagcatattcaactacattgagtgtgtctgtctgtcatttcctcgccgattcctgatttctccttgagccttttcccttgttctccctcggtcggtggtctccacgagaggcggtccgtggcaaaagtgtataaatgttctaaaacatttgaactctaaaacatgcaaaatgaaaaattaaaataaataaacatgaaaattaaaatatattagctgctaaaagttaaacaatactatataatattttgttattagaattcaaaatcacattagttggatttaatttgaacattgcattctttcaataataatttcaataaaaaaagtttccccatgatagtagaaaataataacatatgtatctatctatttatttaactacacatatatagcatttgtttcaactaaaataaatgaatgagcaaagcacctaagtaattggtgtctattatatttatgaagccaatagtttcaaataaattatcatgcataaggaggtattgcaaatgttaaaccttttttgaaacagatattcccagttacagaaattataatttctaatctttcctataagtagaatgatgataattaatataggccatttgtaaataatgttcagattaaaatattctctatttcactagagaagaatgatattaaatgtattatattttatttcccattttgtttgcaccactattctatatccctcagcagtttaaatttgtttcaccatatgtgtgtgtgtttgtatcttaaatatggcactaaaattagaataatttaatataaatctttaggagaaaagatattgaattattttatgttgataggaaaatatcttttaattgtccaagaatactttttcttctattttaggactgatcagacccaggactaatattttatatgtactaattctatgtaccaaaatatgttattatctcatgaattctgtctcaatattgaggtaataaaaatagtccatcatgaactttaaaattaaaataatgattaattaatttttattcatattttgtttgtatgaatggttatacatcacatgtgtgcctggtgactgtgaatgtcaggagaaggtatgaaagccactggaattggaataagagataatatttgagatgttatgtgggtgctgagaattagacgcaagccatcttcaagaatagccagcatactataccactgagtaatccattcatccctcaataattatctttgtagacagtaaatatatttctaaactataaatgaccagaaaaattaatgtattattaatgaagacattcatctcatgtgacacacttcacctgtctaaatcagtaacactctctccactaattaagattttctaagtgcatgacacttactatttctaaagctgtccaatgggggccagtccccagtcagcacccagtgagataatccatgaatgcatttatatcttaggaaaaattcttatctatgtagtatttagaacattttcatgtgaggggataaacaaggaagcacagatgctttctgatagaaactttctctttaattcatctagaaaaaaaaaacctctcaggaaaatctctcttgctctcctcccaatgctctattcagcatcttctccctacttaattctagatctttttctctatgcctccttgctgctgccctgctggctctgctctatgcctccccatgtcacttttctttgctatctcaccgttaccttctctgcctcactctctgccttcttctctgcttctcacatggccaggctctggacaattatagttatatgttacattctcataacacatgatatgtcacatagtttctctcaggctagggatatcacaatgactggccaatgagcaagtggccttgcatgtagctctaagttggtgatggttcccagacagtaagtagccatttggttgaaatttgaggttgggtagtacatgaagactgaattttcttcaaactctggccttgaaatagtaaaacaacacctatgaaaatgacgacctgtatttgtctttagaggcaaccacatattgtctgcagggcctgctttgaatttgctctgaagttagcttgtttgtgtaaaaggaagaatcctatatcagcctgagaaatgtaaaatatcctagcatttcaagtcatcaaaattatatggagagtataaatcatccttctgactattcatagtcatatttgtgtccaccaagtataaaacacactaccaaagggctgtggaaaaaatcgccataactgttcttattagggaggcatagcagtggtacctgaggaagttacagcaacaaccagtcatccagtcaataaccccatggctttgccacttggaggtacccaataatgtttggctttgccgagtaggactccaacaaattcagagggtcaatttttaaatgctggttgtcactgctgaacagtcccattgccctctgcataattccacaatggaaagctttttacactgattgccaatcattaaacagcctactcagcataaacaggtatgatattattctgcattttgttacattactagatgaattcctatttcttcctacaatagtggaactgaaaaaagatacacaatcatactacccctctactaatcttatgacttatatcatttcaattttcagaccataatgcaaactattgaccaaaacatgtgaagatgaaaaatagaaatgtagaataatattacatataaaaagaaaaggcggacttattttgttttatttcttagcatgcatagcaatacatgatttgaggtttatataataaagggacaataaatcttcaagaaacttacccctactgaattaaaatattaaagaaggtcacacatttactcaaatatattagactactgggcaaatagacatgaaaagtagagttaatattgaggtaggccttctgtgaaatgtctaaggaaattatgtttcatacagtgtgtaaccaagtgggaatcatatcagaaagcagtcaaaagcttatattacaagtaacagatgcttggttatatgacctcccagagcttgactgtctatacacaaaaagtggtgttaataaaactgtaatttgggctatgtttttttaaatggcttcaccaacatgaaaggaagggaatgagcatgtcatggatgcttagagattatgcttccagcaagaagaattgagctttggctcttattacagaaacatgacaaggtgtgagttttatttattagaaattatataatattttaagctggggactaaaaattttattgaaacaaacaggcaagggataggcatgtactagaagcaaaaataggatgtcaatgctgtaatgttattttttggaccaaaatagtatttcctatagaaatgacaatgatcttaggttattattcttcataaagatgacaagttcacaagatatcctagttcattaaaatcgttttagtcatttaatagagtgctgtgatagattacacaaaggaaagcacttacgatgagaaataatgatatccacaattattttcttaattcttagaaacattctattgttatatctcaatctcagaagccacttattgctttattattgaaacatatgaaattgtaagttatatattgtctatggtgacatttcaaagaacatgtgacgtacagtgtagcacagataaagaacataactgcagctgaatcagtaactaaacttacatacattaaatctgccatgttggcaacagtgtgtgcactaccaaaggatgtactaatgctcacgacactcccctatgtcaccctttgttcatcattacatcataggtctattttgtttgcttttgaaatctagaccaagtcttttgtgtctttccaagcacagagctcattaatttacctcatagacttgttaaacttcttctggttcatcaattgaatagaaatactcactactaattatgtgagaccctgccagtaccatagcacatggataatttttacataaaacatgcatacaagtaagattattcagactgaacatgaattttagagaaatcaggaaggagtatatgggagtggttggagtgagactagagaaatgtaattaaactataatctcaatacaaagatctactaagcaaaaaacatgaaacattgtcattcaagtgaaacatcagtcttcaaattggaaagatatttttactaggaaaatgtctggtagatggttattatctagaaaacacaaaaattagaaaacggtaaactttaataaaaagaataatacaatgagactacatgaaaagttcttaactaatgaaacaaatatcttgaaacttttttcttaaaagtttaatatcaataaccatcatggaaattcaaattaaaactatttacatattacccctgaaataataactaatacccaataaaaataatataaacaaaaaatggcaatgcatgccatcatggatttgggagagagaatgttcattgcagttctgaatggatactggtgccaccacggtgaaaatctctgtataggtccttccaaaagctgaaaatagacatatcacaagacctgccacacatttttcaagcaaatacccaaaggactctacctgactgcagagacactttctcataaaatattattgttgatctattcataatatctggaaaatagaaacagccaagatgcccatcaactgattaatagatgataaaattattgtacatttcagtgtaatattattcagtttttaagaaaaatgaaattatgtaataagcatgtaaatggatatatcttgaaacaaccattccccattatattacctaaacattgaaagtccaaaatcatatgatctttttagtggatctactaatcttttgctatatgtattttattgaactacccatggatgtgagataattggtaacaacagcacatgggagagcatgggatcattcaaggaagattagagagaatgcattttttaggagataatggaggagcaatagaaaggattaaatgaggttactgatgaaagtgatggttagagaaggcaatatgaggagggataactagcacttagggccttttgaaaaagacatagagaaaatactattgtagaaacttcctataattggtgtatagttatatacaccaaagagctcagatggagttaccctataatggaaatattaactactttttatcactgtgataaaacatcctgaacagagcaacatagattgggaagcatttactttggcttacagttctaacgggataaaaattcatgatgaaagaatgaatatgtcagcaaacagcagtagcaatggcctgagaagcaggtgagagctcacatcttgaagtgtaagaatgtagcagagagaacaaactgcaaatgaccagaaaatgcttttggatcagagcccatacccctctgactgacttctccagaaattctgaacaaataaaactccccaaacagagccataactgaaggtccagtgtctgagactactaggggtatttcttattcaaaccactacaatggggtggggggagcaatcctccaagtaggcactacacacagacaaataaaaactctagtaactggaatggattgacttatttgaattacttgccagtggagctacatagagcacaattattgtatttaaattaccctttatgatcttacaaaacttgacagtaagatcatattgctaaagaaaccacatatttgaatcagggaacatggtgatatctagttgttcttcaactggaaacttcatgctttctgcccagcattcatgttgctggaaagagcaatgtacactaccagtgtagaaattaaatcatcaatcttatcaagatgtggatcctataagttacaataaaaattagcctgataagatatccccaccagaagaatattcacataaatgctatgggagcaacaagctattttctaaattagctttaatcctattctacaagagagaatccatatctagaatagttatagggatcaagaacccatggcttgattggtcataggcccaatgggagatcctaatattattgttctacaaaatgaaaataactcctaatgacttgttgctgcagtaataagttagtatgttgctcaactctcacaagagaagttttgtcttacaataaatggcaattaaagcagccccacaagatttatatcataccgatctcctcatggcctatgcatctagaagctaggaaacaaagaggaccctaagagagacatacatggtccccctggagaaggggaagggggcaagacctccaaagctaattgggagcatgggggaggggagagggagttagaagaaagagaaggggataaaaggagggagaggaggacaagagagagaaggaagatctagtcaagagaagatagaggagagcaagaaaagagataccatagtagagggagccttgtatgtttaaatagaaaactggcactagggaattgtccaaagatccacaaggtccaactaataatctaagcaatagtcgagaggctaccttaaaagcctttctctgataatgagattgatgactaccttatataccatcctagagccttcatccagtagctgatggaagcagaagcagacatctacagctaaacactgagctagttgcagacagggaggagtgatgagcaaagtcaagaccaggctggagaaacacacagaaacagcagacctgaaaaaaatgttgcacatggaccccagactgatagctgggagtccagcataggacttttctagaaaccctgaatgaggatatcagtttggaggtctggttaatctatggggacactggtagtggatcaatatttatccctagttcatgactggaatttgggtacccattccacatggaggaattctctgtcagcctagacacatgggggaggttctaggtcctgctccaaataatgtgttagactttgaagaactcccttgagaagactcaccctccctggggagcagaaaggggatgggatgagggttggtgagggacaggagaggaggggagggtgagggaactgggattgacaagtaaatgatgcttgtttctaatttaaatgaataaaggaaaagtaaaagaagaaaagaaaacaggccaaaagattataaaagacagaggtggtgggtgactataaagaaacactattatctaaataaaaatatgtcagaagcacacatgaacttatagtgtttatgaaagtatgtataataactacataatctcaagccaagaaaaaaatatcatctttcagtgatgaaggtgattttatttctcccagaattaaagccaaagacctaatgaaagtaattatcttcaaaaggttgaaaatacatactttgcaatacacagatctgcctagaaatctcatgttcacaatacacatgatgctcaattgaattccattcaatgttacagtttagataaacagtttgtagataaactcacaatgtatcatttctttttattttttgaccaaacagcttctcatctgttattcagaataattcctcgatggcaggatatccatcccaattgggggaaggggagaatttgaagaaaacctagaccacatacatatttgccattgggaaacaaagtctaaaatgatgttgttcacatcttctctactagtcctctccccgtcccaaagaaccttggtatatgtgcctcattttacagagagaggaaagcaggaactgagcatcccttacttgccatcctcaacccaaaatttgcatcattgctcagctctgcccttctcatatgacagttacaagtcaaggcttccaaagtccctctgtcatgtttggtgtcaatagtttatacagatgacttcatgtcttcatatctaatgtcttatatagattaatattaaacaatgttatttctctaaccacattttaaattaatttaaaaatccattaattgtgtctataaaatgcagacagagtgctgagacacaatataagcctgatgatctgaatttgaaactcacacccaccacatggagaatcaacttccaaaaattttcctattacttccacacttacaccattgtacaaacacaataataatgaacaaaatgaaatgaaataaaaaattaagtctctgtaggtaatgctactgtgcagcaaaagtaaaaatggcagcttaagcttgctttatggttacactttaccatcttccattaattataaggacttcaatcatggcagaactatgctgttattgtctcagtgtaacctaaccaggtgttccagatgttcttaatgtggacacctaaactatttgatatttgggttaagatctttccctctttcagaagaaacctcaggacagagggaatcttgtcttttaattttgagtctgtagactttttccatttcaaatatacatgaaacaagtgatgaagaaaattaatcaaaaggtgggaattgcaatgatattaggttcaatattaagcttcaatattatcatggaatcgcctgttatacactgagtgtttggcaataagggatttttagaagaaggagtttttattctcaacaggttccttaagtttagctcaaataaatctaagcaatccactctagaattaaatagtttcctaagggcacagctatgaatagagctcaatttacatataaaattttgttcaccatttatgtcattccagttttcattagtacaaggaaaatacaaaatatttagatgtcaatatcaagtgaatagttcatctccttttttaatatatatcacctaaatcaccattttctcagaaaaatctggcctgaagttctgtctggaacttcaacatgaaaaatatgcacagcttgctattataaatcctagttgatttttaagattcatgtctggtgtctgactcagaggggccagaggctagacaaatattttttgaatcttcattgtgaagatttttaatgattattttaatataaataacaaagatgatggataatgtaactttgtacagttcatagacgctgaactactttgtgcttaaaatgttagttccctatcataaatgataggtgataagtgtatgtttaatactttccctctgagctatattcatgtactagagaattattttaaacatgaaaagactgtgtttatagtctcagctcctgagaactggtccaaccttaggcaggtgaatgccaggagcaacgtttttcttctacagaggatgctttgctgccaagcaacctggttgtgtggaaatgttccttttttaatcaagtttaaagggtcttcatcatgctgttgctccacatattttcaggttagagcttggtccttggagtattatcttttaccagaaaattcatagtattctttcaataactaacaactaaacttttcgataaaaaagaattggaatttcaattttaaagcctgagtaaaattcttgtgaatcaggatattttattttaagtcttatcttttaaaaagttattttattttttaaaaaattataatatactttcataatttccctccttcacttttctttacaaacacttctatagatcaccatgtgtttttttttttacatttatggcctctttctgttcattgttattacatacaaatagtcttgcctatagaagaacaccacaatttgttacctgataacaaattatcaacccttaaaacctacaaactattgatattactgaaaagactatacttatagatgtaaagatatatgtgtgtgcacatatatagatacacatatatgtaggatttttaattttagattttagacatcaaaattatttatatgactgagaaactagacactataaatgagcattcagtattcaacaccgtgattttagatattgtcacaatgacagaaaattttcttatagaaaattttaagttttgtgattgctctgtgcacttagtgaagtctcacagaaaaagaatcatagtatttttagtttataataaaaagtacatataattaaaatggttggcacaaaacaacatttgagcatttttcctatttactatcaagtagtatcattttgaaataataatttgactagtttcaaaaatgaaaacaaaatttaaactaaatgcctaatctagcctgataacatttttatgaatgaaattattcaatagtgttatcaattaggggcccaaaacttttcctaaaataaaacttttaatttttttccatttttatttaaattagaaacaaaattgttttacatgtaaatcagagtttcctcaccctccccttctccctgtccctcactaacaccctacttgtcccataccatttctgctccccagggagggtgaggccttccatggggaaacttcagagtctgtctatcctttcggatagggcctaggccctcacccatttgtctaggctaaggctcacaaagtttactcctatgctagtgataagtactgatctactacaagagacaccatagatttcctaggcttcctcactgacacccatgttcatggggtctggaacaatcatatgctagtttcctaggtatcagtctggggaccatgagctcccccttgttcaggtcaactgtttctgtgggtttcaccaccctggtcttgactgctttgctcatcactcctccctttctgtaactgggttccagtacaattccgtgtttagctgtgggtgtctacttctactttcatcagcttctgggatggagcctctaggatagcatacaattagtcatcatctcattatcagggaagggcatttaaagtagcctctccattgttgcttggattgttagttggtgtcatctttgtagatctctggacatttccctagtgccagatatctctttaaacctacaagactacctctattatggtatctcttttcttgctctcgtctattcttccagacaaaatcttcctgctcccttatattttcctctcccctcctcttctccccttctcattctcctagatccatcttcccttcccccatgctcccaagagagatgttgctcaggagatcttgttccttaacccttttcttggggatctgtctctcttagggttgtccttgtttcctagcttctctggaagtgtggattgtaagctggtaatcatttgctccatgtctaaaatccatatatgagtgatgtttgtctttttgtgactgggttacctcactcaaaatggtttcttccatatgtctgtggatttcaatagcacaaacaacatacagtatcttggggcaacactaaccaaacaagtgaaagaccagtatagcaagaactttgagtttaaagaaagaaattaaagaagataccagaaaatggaaagatctcccatgctctttgataggcagaatcaacatagtaaaaatggcaatcttgccaaaatccatctacagactcaatgcaatccccattaaataccagcacacttcttcacagacctgaaagaataatacttaactttatatggagaaacaaaagacccaggataggccaaacaaccctgtacaatgaaggcacttccagaggcatccccatccctgacttcaagctctattatagagtaataatcctgaaaacagcttggtaatggcacaaaaatagacaggtagaccaatggaattgagttgaaaaccctgatattaacccacatatctatgaacacctgactttgacaaagaagctaaggttatacaatgtaagaaagaaagcatcttcaacaaatcgtgctggcataactggatgctggcatgtagaagactgcagatagatccatgtctaatgccatgcacaaaacttaagtccaaatggatcaaaaacctcaacataaatccagccacactgaacctcatagaagagaaagtgggaagtatccttgaataaattggtacaggagaccacatcttgaacttaacaccagtagcacagacaatcagatcaataatcaataaatgggacctcctgaaactgagaagcttctgtaaggcaatggataagtcaacaggacaaaatggcagcccacggaatgggaaaagatattcaccaatcctatatctgacagagggctgctctctatttgcaaagaacacaataagctagtttttaaaacaccaattaatccgattataaagttgggtagagaactaaataaagaattgttaacagagcaatctaacttggcagaaagacacataagaaagtgctcaccat